(19) United States
(12) Patent Application Publication
(10) Pub. No.: US 2024/0190834 A1
(43) Pub. Date: Jun. 13, 2024

(54) METHODS FOR EXTRACTION, PROCESSING, AND PURIFICATION OF MINOR CANNABINOID COMPOUNDS FROM COMPLEX MIXTURES

(71) Applicant: Nectar Health Sciences Inc., Victoria (CA)

(72) Inventors: Jay Van Der Vlugt, Ottawa (CA); Tony Durst, Victoria (CA)

(73) Assignee: Nectar Health Sciences Inc., Victoria, BC (CA)

(21) Appl. No.: 18/256,033

(22) PCT Filed: Dec. 6, 2021
(86) PCT No.: PCT/CA2021/051742

§ 371 Date: Jun. 5, 2023

Related U.S. Application Data
(60) Provisional application No. 63/123,027, filed on Dec. 9, 2020.
(60) Provisional application No. 63/121,557, filed on Dec. 4, 2020.

(51) Int. Cl. C07D 311/80; C07C 37/84; C07C 211/62; C07C 211/63; C07D 211/12; C07D 213/74; C07D 453/02; C07D 487/04

(52) U.S. Cl. CPC C07D 311/80; C07C 37/84; C07C 211/62; C07C 211/63; C07D 211/12; C07D 213/74; C07D 453/02; C07D 487/04

(57) ABSTRACT

Disclosed are methods for separating, recovering, and purifying CBGA, CBDVA, THCVA, CBCVA, and CBCA from dewatered and desolventized crude complex extracts or mixtures of metabolites, cannabinoids, and Cannabis phytochemicals. The methods comprise solubilizing the extracts or mixtures of cannabinoids in a selected solvent, adding a selected amine to precipitate a CBGA-amine or CBDVA-amine or THCVA-amine or CBCVA-amine or CBCA-amine salt therefrom, dissolving the recovered amine salt in a selected solvent, and adding a selected antisolvent to recrystallize a purified amine salt therefrom. The recrystallized amine salt may be decarboxylated to form a mixture of CBG or CBDV or THCV or CBCV or CBC and amine. The cannabinoid and amine mixture may be acidified to separate the amine from CBG or CBDV or THCV or CBCV or CBC. The recovered CBG or CBDV or THCV or CBCV or CBC may then be concentrated.

### CROSS-REFERENCE

This application claims the benefit of U.S. Provisional Patent Application No. 63/121,557 filed Dec. 4, 2020. This application also claims the benefit of U.S. Provisional Patent Application No. 63/123,027 filed Dec. 9, 2020.

### TECHNICAL FIELD

Various embodiments disclosed herein generally relate to methods for processing and separating mixtures of cannabinoid compounds extracted from Cannabis biomass feedstocks. More specifically, this disclosure pertains to methods for separating, precipitating, and purifying one or more of cannabigerol, cannabidivarin, tetrahydrocannabivarin, cannabichromevarin, or cannabichromene compounds from Cannabis extracts.

BACKGROUND

Cannabis is a genus of flowering plants in the Cannabaceae family. Cannabis sp. are known to produce at least 113 distinct cannabinoids and over 50 terpenes that are concentrated in viscous resins produced in plant structures known as glandular trichomes. Trichomes are located at about the axial growing tips of Cannabis plants. Perhaps the most recognized cannabinoids are tetrahydrocannabinol (THC) and cannabidiol (CBD). It is well known that THC has significant but temporary psychoactive effects (i.e., hallucinogenic) on mammalian physiology and for this reason, various formats of Cannabis sp. plant materials and extracts are consumed for recreational use. It is also well known that CBD does not have psychoactive effects (i.e., hallucinogenic) but does have significant calming and pain relief effects. As an aggregate group of compounds, Cannabis terpenes are known to provide characteristic distinct aromas and flavors. It is also known that terpenes interact with cannabinoids to modulate the physiological effects of cannabinoids.

It is also well known that fiber-type Cannabis, commonly known as hemp, has relatively high levels of CBD with very low levels or no levels of THC and consequently, is considered to have no or only minimal psychoactive and/or anxiogenic effects. The term “hemp” derives its definition from legal and/or regulatory distinctions for fiber-type Cannabis strains and cultivars that stably and reproducibly have less than 0.3% THC in the USA.

Cannabinoid compounds used for both recreational and medicinal purposes are almost exclusively crude extracts that have been solubilized and recovered from Cannabis plant biomass with one of aqueous solvents, organic solvents, supercritical CO₂, and the like as a first processing step. The resulting crude extracts generally comprise complex mixtures of cannabinoids, terpenes, flavonoids, polyphenols, alkaloids, steroids, and other phytochemicals, which vary with the type of solvent selected for the extraction process. Numerous processes are known for use to refine crude Cannabis extracts to separate out undesirable phytochemical components and to concentrate the cannabinoid and terpene components.

The most commonly known and widely used Cannabis extraction methods are based on the use of organic solvents. Some drawbacks associated with such methods include poor or inconsistent yields and high costs associated with extraction and purification of extract and toxicity of some of the extraction solvents. Government regulations and security for Cannabis plants are also an important consideration that adds to the overhead cost of producing extracts containing cannabinoid compounds.

Consequently, the most commonly available and studied cannabinoids are cannabidiol (CBD), cannabidiolic acid (CBDA), tetrahydrocannabinol (THC), and tetrahydrocannabinolic acid (THCA) for which considerable therapeutic and recreational knowledge and understanding have been developed. However, because of the difficulty in separation, recovery and purification of minor cannabinoids from the CBD, CBDA, THC, and THCA moieties present in Cannabis biomass using known Cannabis extraction processes, not much is known regarding the potential therapeutic benefits that the minor psychoactive and non-psychoactive cannabinoids might provide. Based on research data with chemically synthesized minor cannabinoids, it appears, for example, that cannabigerol (CBG) may have useful properties as one or more of a vasodilator, a neuron protectant, an agonist of certain cytokines, an antagonist of certain other cytokines, and a blocker of certain receptors associated with cancer cell growth. Cannabichromene (CBC) has provided indications that its potential therapeutic benefits include antimicrobial and antiviral properties, anti-inflammatory properties, analgesic effects, and inhibition of the growth of cancerous tumors. Cannabidivarin (CBDV) is a non-psychotropic cannabinoid that appears to affect the neurochemical pathways of capsaicin receptors, neurobehavioral issues, and other neurological disfunctions. Tetrahydrocannabivarin (THCV) is a non-psychotropic cannabinoid that is considered to have anti-inflammatory, neuroprotective, and anticonvulsive properties. Cannabichromevarin (CBCV) is a non-psychotropic cannabinoid that considered to have anti-convulsive properties and may be useful for treatment of epilepsy.

A significant challenge in assuring the delivery of consistent reproducible quality and content of extracts, including cannabinoid extracts from Cannabis, is due to natural variations of endogenous phytochemicals that occur in plants. The chemical “fingerprint” of a particular botanical species can vary widely depending on the age of the plant, time of harvest, soil conditions, weather conditions, and a myriad of other factors. It is known that botanicals with very different phytochemical profiles will have different therapeutic effects, even if the botanicals are recovered from the same plant species. Standardization of botanical extraction processes facilitate the batch-to-batch reproducibility of a final product. A standardized extract has a selected concentration of a marker compound that is known to a high degree of accuracy, and because both the amount of botanical material that is extracted and the amount of a carrier that may be added can be varied, it is possible to compensate for natural variability in the plant material. Also, if endogenous phytochemical active components of a standardized botanical extract are administered to patients in known quantities, then the treatments following prognosis of a disease can be monitored. Therefore, there is a need for standardized and reproducible extracts of botanicals, including extracts derived from Cannabis.

### SUMMARY

The embodiments of the present disclosure generally relate to methods for separating, recovering, and purifying minor cannabinoid cannabigerol (CBG), cannabidivarin (CBDV), Δ9-tetrahydrocannabivarin (THCV), cannabichromevarin (CBCV), or cannabichromene (CBC) compounds from de-watered and/or desolventized crude extracts comprising complex mixtures of compounds. The de-watered and/or desolventized crude extracts may have been recovered from Cannabis plant biomass feedstocks. The de-watered and/or desolventized crude extracts may have been recovered from fermentation broths wherein selected microorganisms were cultured to produce selected phytochemicals.

According to an embodiment of the present disclosure, Cannabis biomass feedstocks may be processed with one or more solvents selected from methanol, ethanol, propanol, isopropanol, butanol, C3-C7 alkanes, low boiling point (b.p.) petroleum ethers, ethyl acetate, acetone, dichloromethane, 1,4-dioxane, tetrahydrofuran, acetonitrile, toluene, methyl tert-butyl ether, supercritical CO₂, and subcritical CO₂ to recover therefrom a crude extract solution in the selected solvent. According to an aspect, the crude extract solution may be desolventized by removal of the extractant solvent to thereby produce a concentrated crude extract. The concentrated crude extract may be dried to thereby produce an oil form or a resin form or a solid form, depending on which type of extractant solvent was selected.

According to another embodiment, fermentation broths wherein genetically modified microorganisms have been cultured to produce target cannabinoid phytochemicals, may be processed to separate and recover therefrom complex mixtures of fermentation metabolites and phytochemicals. The processing may include separating the cultured microorganisms from the fermentation broths prior to separation and recovery of complex mixtures of fermentation metabolites and phytochemicals. The processing may include steps to recover metabolites and phytochemicals from the cultured microorganisms. The complex mixtures may be processed by one or more of dewatering steps, desolventizing steps, drying steps, filtration steps including microfiltration steps, extraction with supercritical CO₂ steps, and the like known to those skilled in this art, to produce a concentrated crude complex mixture in an oil form or a solid form or a dry form.

Some embodiments according to the present disclosure pertain to methods for forming, recovering, and purifying cannabigerolic acid-amine (CBGA-amine) salts, cannabigerolic acid (CBGA), and cannabigerol (CBG) from solvent-solubilized crude complex extracts or from solvent-solubilized crude complex mixtures. A selected amine may be added to a solvent-solubilized crude extract or to a solvent-solubilized crude complex mixture to precipitate therefrom a CBGA-amine salt. The precipitated CBGA-amine salt may be washed one or more times with a selected alkane solvent for example, heptane, and then dried to produce a washed CBGA-amine salt. The washed CBGA-amine salt may be resolubilized in a suitable solvent disclosed herein and then, recrystallized to produce a purified CBGA-amine salt. The purified CBGA-amine salt may resolubilized, followed by separating the amine therefrom, and recovering a highly purified CBGA. The CBGA may be decarboxylated to produce a highly purified CBG that may then be crystallized if so desired.

According to an aspect, the purified CBGA-amine salt may be decarboxylated by adding and dissolving the CBGA-amine salt into a sodium carbonate solution and mixing the solution at about 100° C. for about 4 hr to thereby form an oil comprising CBG and the amine. The decarboxylated CBG may be dissolved into a selected alkane solvent or alternatively, may be dissolved into a low-boiling petroleum ether. The dissolved amine may then be partitioned from the dissolved CBG by the addition of aqueous HCl thereby forming an aqueous layer containing the amine therein, and an organic layer containing the CBG therein. After separation and removal of the aqueous layer, the solvent may then be removed from the organic layer thereby producing a highly purified CBG.

Some embodiments according to the present disclosure pertain to methods for forming, recovering, and purifying Δ9-tetrahydrocannabivaric acid-amine (THCVA-amine) salts, Δ9-tetrahydrocannabivaric acid (THCVA), and Δ9-tetrahydrocannabivarin (THCV) from solvent-solubilized crude Cannabis extracts and mixtures according to the present disclosure. A selected amine may be added to a solvent-solubilized crude Cannabis extract to precipitate therefrom a THCVA-amine salt. The precipitated THCVA-amine salt may be washed one or more times with a selected alkane solvent for example, heptane, and then dried to produce a washed THCVA-amine salt. The washed THCVA-amine salt may be resolubilized in a suitable solvent disclosed herein and then, recrystallized into a purified THCVA-amine salt. The purified THCVA-amine salt may be resolubilized and the amine separated therefrom to produce a highly purified THCVA. The THCVA may be decarboxylated to produce a highly purified THCV oil that may then be crystallized if so desired.

According to an aspect, the purified THCVA-amine salt may be decarboxylated by adding and dissolving the THCVA-amine salt into a sodium carbonate solution and mixing the solution at about 100° C. for about 4 hr to thereby form an oil comprising THCV and the amine. The decarboxylated THCV may be dissolved into a selected alkane solvent or alternatively, may be dissolved into a low-boiling petroleum ether. The dissolved amine may then be partitioned from the dissolved THCV by the addition of aqueous HCl thereby forming an aqueous layer containing the amine therein, and an organic layer containing the THCV therein. After separation and removal of the aqueous layer, the solvent may then be removed from the organic layer thereby producing a highly purified THCV.

Some embodiments according to the present disclosure pertain to methods for forming, recovering, and purifying cannabidivaric acid-amine (CBDVA-amine) salts, cannabidivaric acid (CBDVA), and cannabidivarin (CBDV) from solvent-solubilized crude Cannabis extracts according to the present disclosure. A selected amine may be added to a solvent-solubilized crude Cannabis extract to precipitate therefrom a CBDVA-amine salt. The precipitated CBDVA-amine salt may be washed one or more times with a selected alkane solvent for example, heptane, and then dried to produce a washed CBDVA-amine salt. The washed CBDVA-amine salt may be resolubilized in a suitable solvent recrystallized to produce a purified CBDVA-amine salt. The purified CBDVA-amine salt may be resolubilized and the amine separated therefrom to produce a highly purified CBDVA. The highly purified CBDVA may be decarboxylated to produce highly purified CBDV oil that may then be crystallized if so desired.

According to an aspect, the purified CBDVA-amine salt may be decarboxylated by adding and dissolving the CBDVA-amine salt into a sodium carbonate solution and mixing the solution at about 100° C. for about 4 hr to thereby form an oil comprising CBDV and the amine. The decarboxylated CBDV may be dissolved into a selected alkane solvent or alternatively, may be dissolved into a low-boiling petroleum ether. The dissolved amine may then be partitioned from the dissolved CBDV by the addition of aqueous HCl thereby forming an aqueous layer containing the amine therein, and an organic layer containing the CBDV therein. After separation and removal of the aqueous layer, the solvent may then be removed from the organic layer thereby producing a highly purified CBDV.

Some embodiments according to the present disclosure pertain to methods for forming, recovering, and purifying cannabichromevarinic acid-amine (CBCVA-amine) salts, cannabichromevarinic acid (CBCVA), and cannabichromevarin (CBCV) from solvent-solubilized crude Cannabis extracts according to the present disclosure. A selected amine may be added to a solvent-solubilized crude Cannabis extract to precipitate therefrom a CBCVA-amine salt. The precipitated CBCVA-amine salt may be washed one or more times with a selected alkane solvent for example, heptane, and then dried to produce a washed CBCVA-amine salt. The washed CBCVA-amine salt may be resolubilized in a suitable solvent disclosed herein and then recrystallized to produce a purified CBCVA-amine salt. The purified CBCVA-amine salt may then be resolubilized and the amine recovered therefrom to produce a highly purified CBCVA. The highly purified CBCVA may be decarboxylated to produce a highly purified CBCV oil that may then be crystallized if so desired.

According to an aspect, the purified CBCVA-amine salt may be decarboxylated by adding and dissolving the CBCVA-amine salt into a sodium carbonate solution and mixing the solution at about 100° C. for about 4 hr to thereby form an oil comprising CBCV and the amine. The decarboxylated CBCV may be dissolved into a selected alkane solvent or alternatively, may be dissolved into a low-boiling petroleum ether. The dissolved amine may then be partitioned from the dissolved CBCV by the addition of aqueous HCl thereby forming an aqueous layer containing the amine therein, and an organic layer containing the CBCV therein. After separation and removal of the aqueous layer, the solvent may then be removed from the organic layer thereby producing a highly purified CBCV.

Some embodiments according to the present disclosure pertain to methods for forming, recovering, and purifying cannabichromenic acid-amine (CBCA-amine) salts, cannabichromenic acid (CBCA), and cannabichromene (CBC) from solvent-solubilized crude Cannabis extracts according to the present disclosure. A selected amine may be added to a solvent-solubilized crude Cannabis extract to precipitate therefrom a CBCA-amine salt. The precipitated CBCV-amine salt may be washed one or more times with a selected alkane solvent for example, heptane, and then dried to produce a washed CBCA-amine salt. The washed CBCA-amine salt may be resolubilized in a suitable solvent disclosed herein and recrystallized into a purified CBCA-amine salt. The purified CBCA-amine salt may then be resolubilized and the amine recovered therefrom to produce a highly purified CBCA. The highly purified CBCA may be decarboxylated to produce a highly purified CBC oil that may then be crystallized if so desired.

According to an aspect, the purified CBCA-amine salt may be decarboxylated by adding and dissolving the CBCA-amine salt into a sodium carbonate solution and mixing the solution at about 100° C. for about 4 hr to thereby form an oil comprising CBC and the amine. The decarboxylated CBC may be dissolved into a selected alkane solvent or alternatively, may be dissolved into a low-boiling petroleum ether. The dissolved amine may then be partitioned from the dissolved CBC by the addition of aqueous HCl thereby forming an aqueous layer containing the amine therein, and an organic layer containing the CBC therein. After separation and removal of the aqueous layer, the solvent may then be removed from the organic layer thereby producing a highly purified CBC.

### BRIEF DESCRIPTION OF THE DRAWINGS

The present invention will be described in conjunction with reference to the following drawings in which:

FIG. 1A is a chart showing a linear calibration curve for cannabidivarin (CBDV);

FIG. 1B is a chart showing a linear calibration curve for tetrahydrocannbidivarin (THCV);

FIG. 1C is a chart showing a linear calibration curve for cannabidiol (CBD);

FIG. 2A is a chart showing a linear calibration curve for cannabigerol (CBG);

FIG. 2B is a chart showing a linear calibration curve for cannabidiolic acid (CBDA);

FIG. 20 is a chart showing a linear calibration curve for cannabigerolic acid (CBGA);

FIG. 3A is a chart showing a linear calibration curve for cannabinol (CBN);

FIG. 3B is a chart showing a linear calibration curve for Δ⁹-tetrahydrocannabinol (Δ⁹-THC);

FIG. 3C is a chart showing a linear calibration curve for Δ⁸-tetrahydrocannabinol (Δ⁸-THC);

FIG. 4A is a chart showing a linear calibration curve for cannabichromene (CBC);

FIG. 4B is a chart showing a linear calibration curve for (-)-Trans-Δ⁹-tetrahydrocannabinolic acid (THCA);

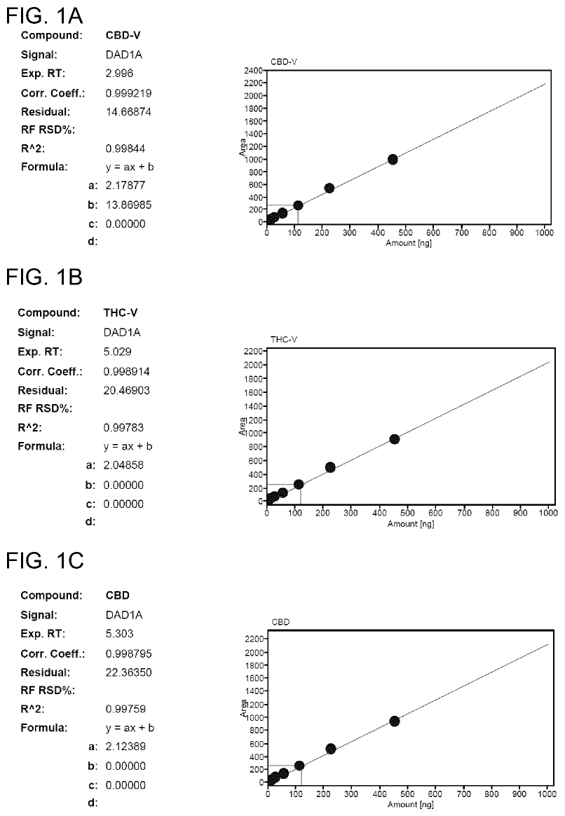

FIG. 5 is an HPLC chromatogram showing separation of a standardized reference mixture of the eleven cannabinoid phytochemicals shown in FIGS. 1A-1B;

FIG. 6 is an is an HPLC chromatogram showing the cannabinoid content of a solvent-solubilized crude hemp extract solution from Example 2;

FIG. 7 is an HPLC chromatogram showing the cannabinoid content of a washed solid CBGA-Hunig's base salt precipitated from the solvent-solubilized crude hemp extract solution shown in FIG. 6 (Example 2);

FIG. 8 is an HPLC chromatogram showing the cannabinoid content of the depleted extract after separation therefrom of the CBGA-Hunig's base salt shown in FIG. 7; (Example 2);

FIG. 9 is an HPLC chromatogram showing the purified solid CBG that was produced from the CBGA-Hunig's base salt in Example 3);

FIG. 10 is an HPLC chromatogram showing the purified solid CBGA that was produced from the CBGA-Hunig's base salt in Example 3);

FIG. 11 is an HPLC chromatogram showing the cannabinoid content of a standardized crude extract produced from a dry powdered hemp kief sample in Example 6.

FIG. 12 is an HPLC chromatogram showing the cannabinoid content of the crude CBGA-Hunig's base amine salt precipitated from the standardized crude extract in Example 6;

FIG. 13 is an HPLC chromatogram showing the cannabinoid content of a CBGA-depleted standardized crude extract in Example 6;

FIG. 14 is an HPLC chromatogram showing the cannabinoid content of the purified recrystallized CBGA-Hunig's base amine salt in Example 6;

FIG. 15 is an HPLC chromatogram showing the cannabinoid content of the decarboxylated CBG oil produced in Example 6;

FIG. 16 is an HPLC chromatogram of an unfiltered HPLC-methanol control sample in Example 6;

FIG. 17 is an HPLC chromatogram of a filtered HPLC-methanol control sample showing an unknown anomaly associated with the filter labeled as “THC-A in Example 6;

FIG. 18 is an HPLC chromatogram showing the cannabinoid content of the crude CBGA-N,N-diisopropylethylamine amine salt (Hunig's base) precipitated from a standardized crude extract solution in Example 7;

FIG. 19 is an HPLC chromatogram showing the cannabinoid content of the decarboxylated CBG oil produced in Example 7;

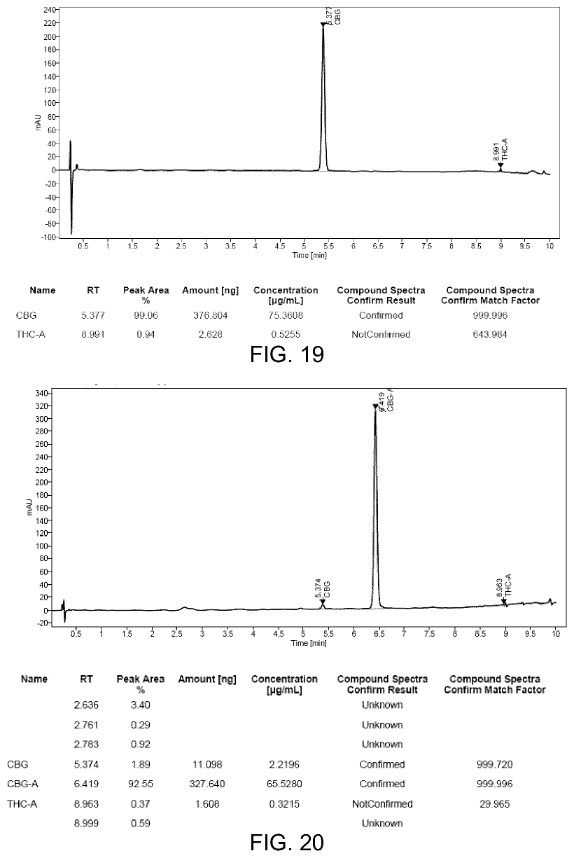

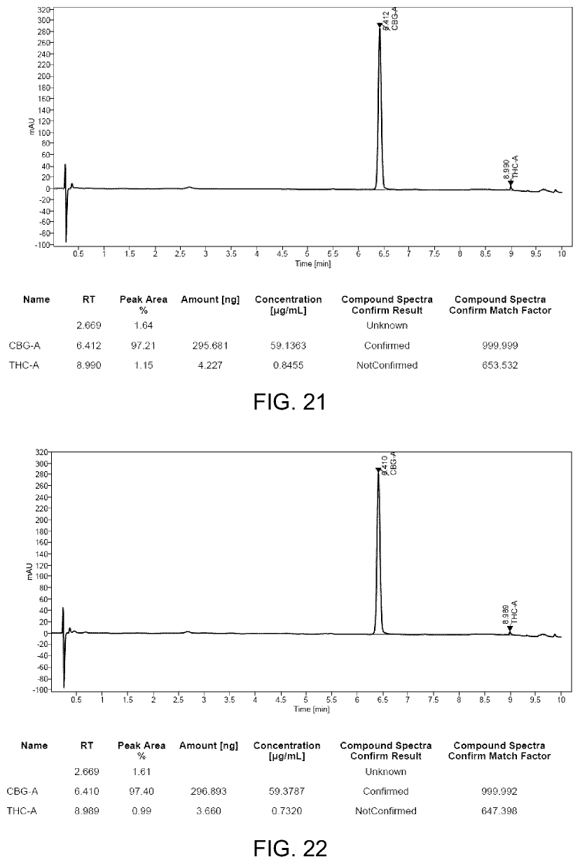

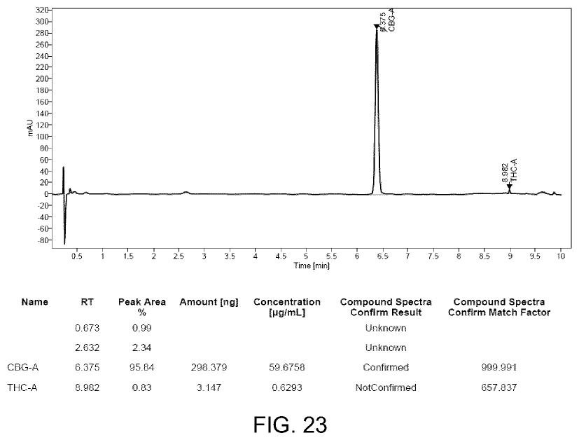

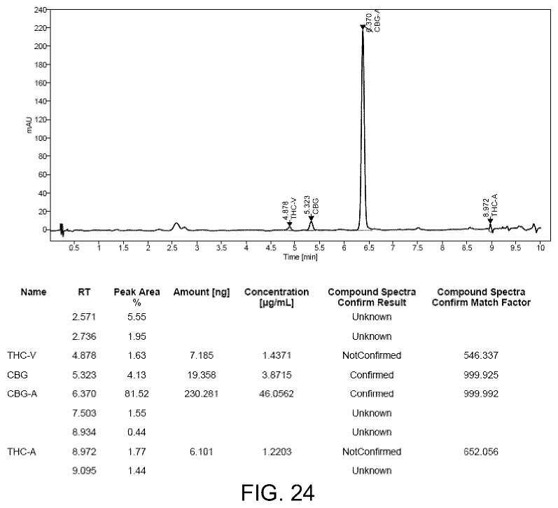

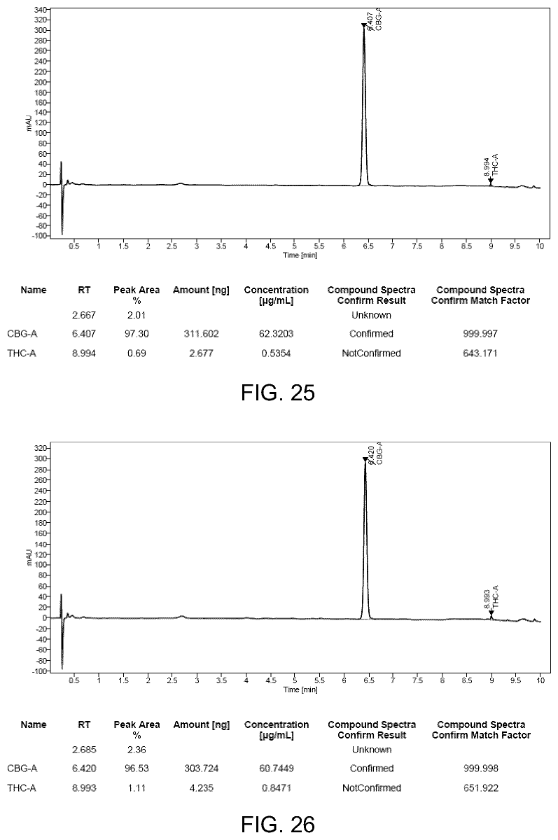

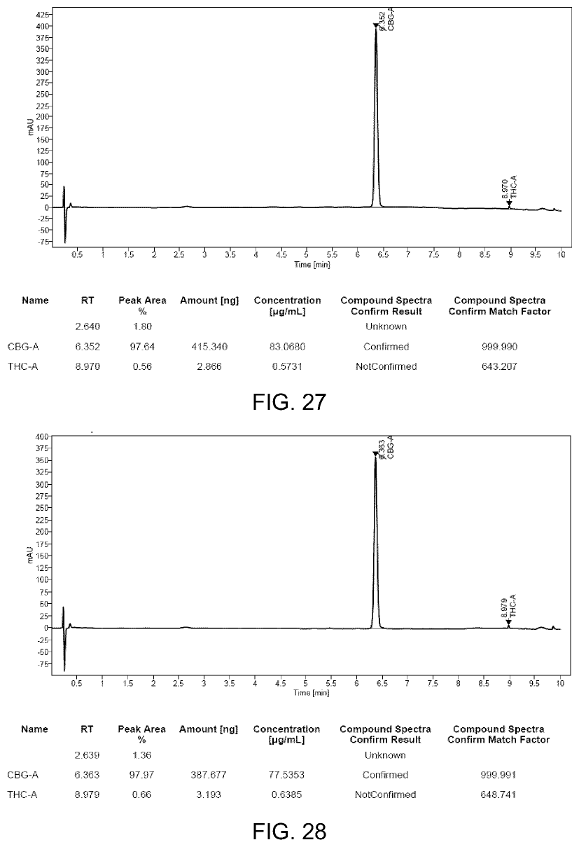

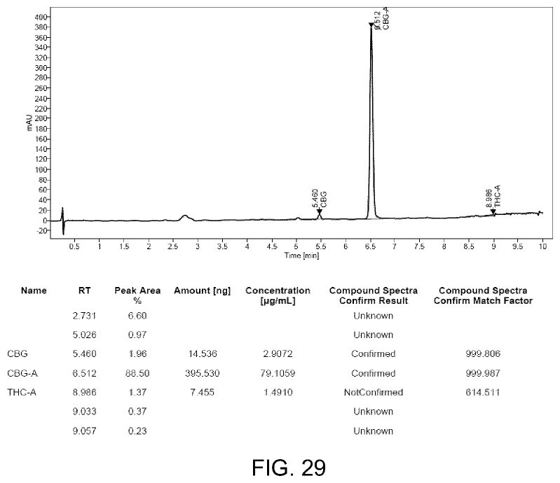

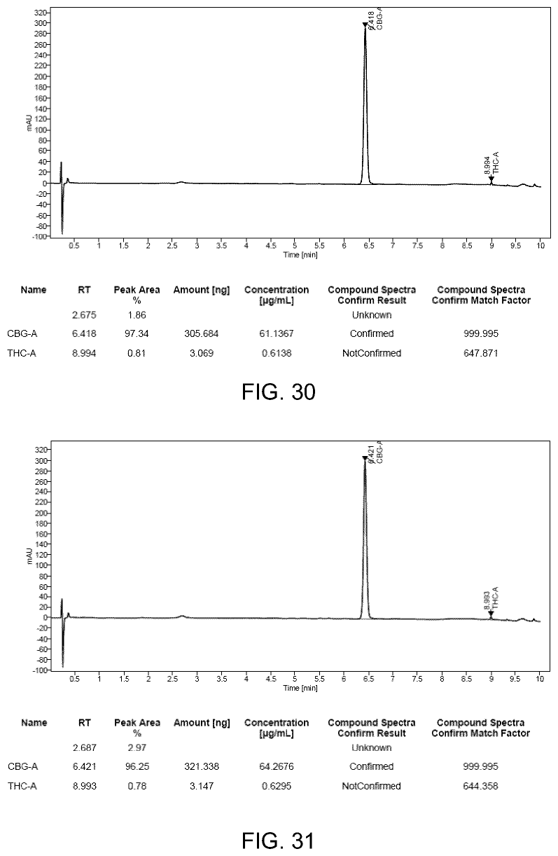

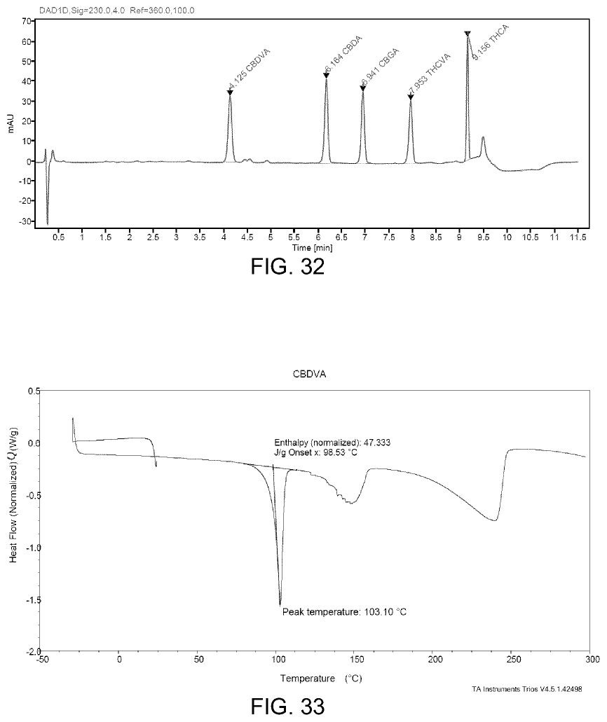

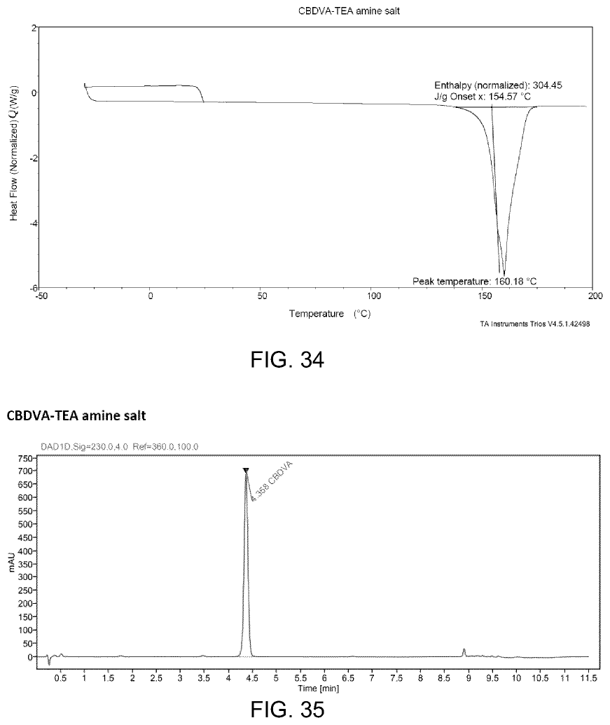

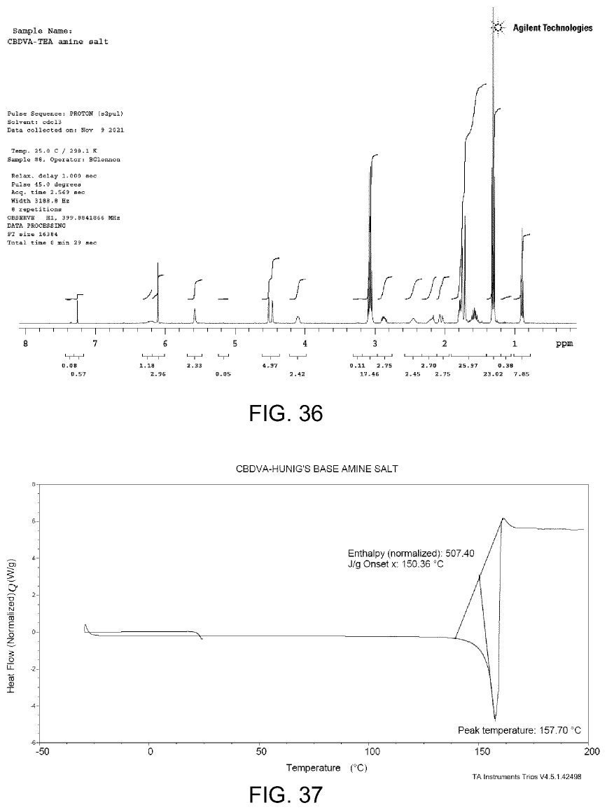

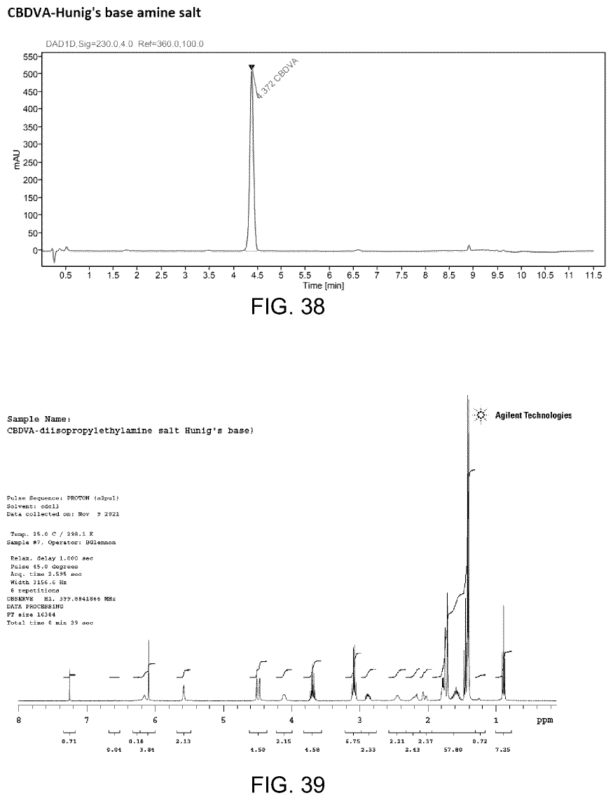

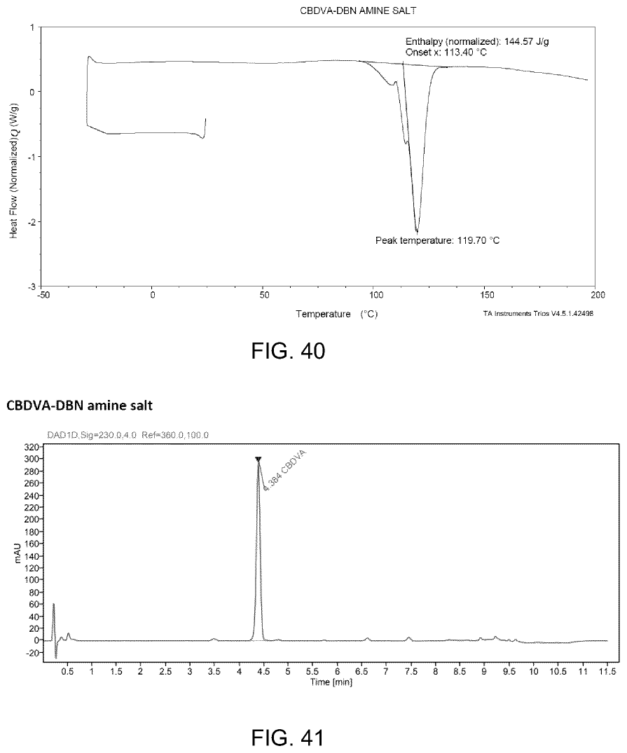

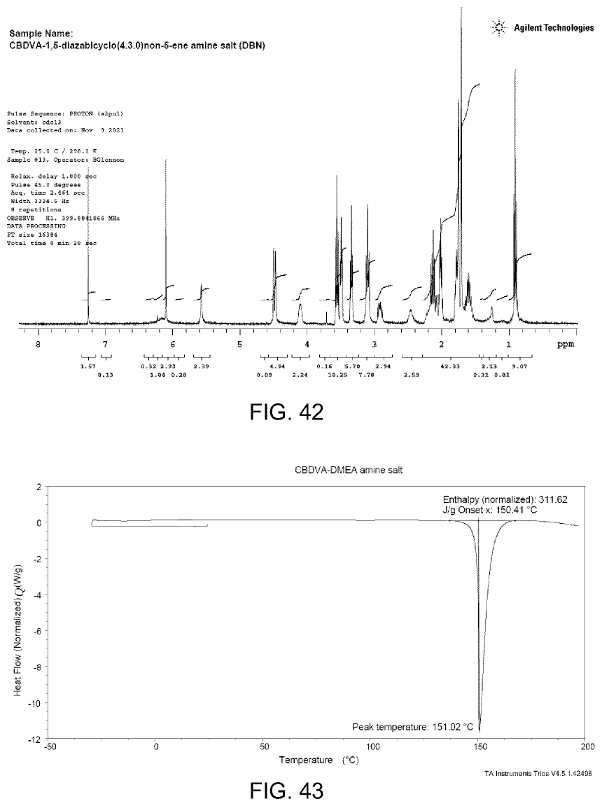

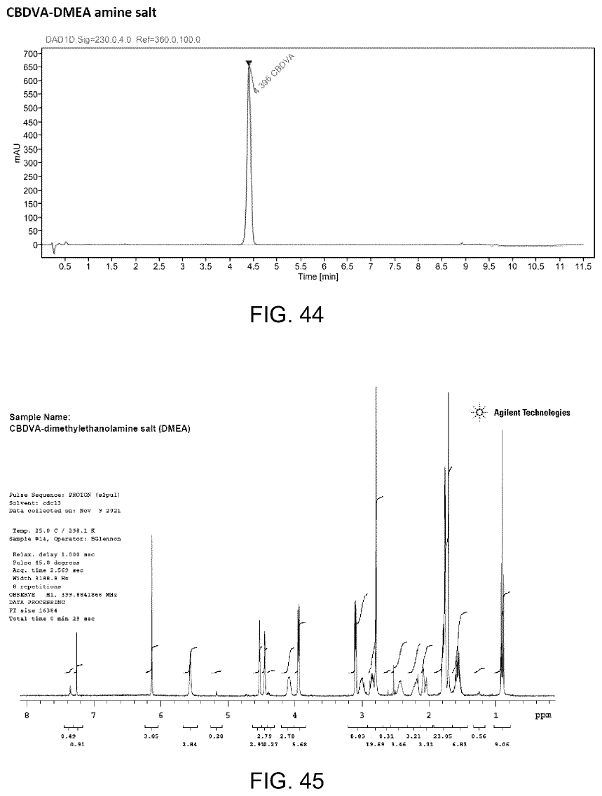

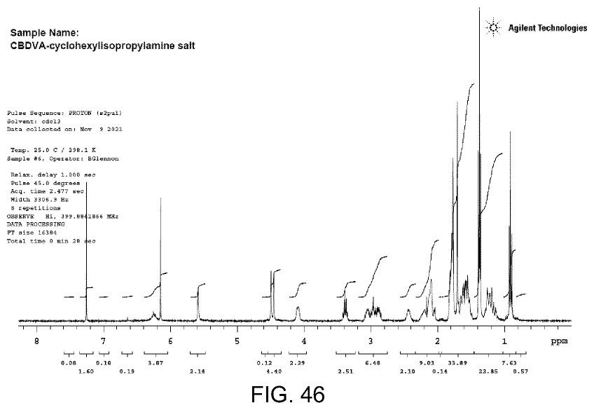

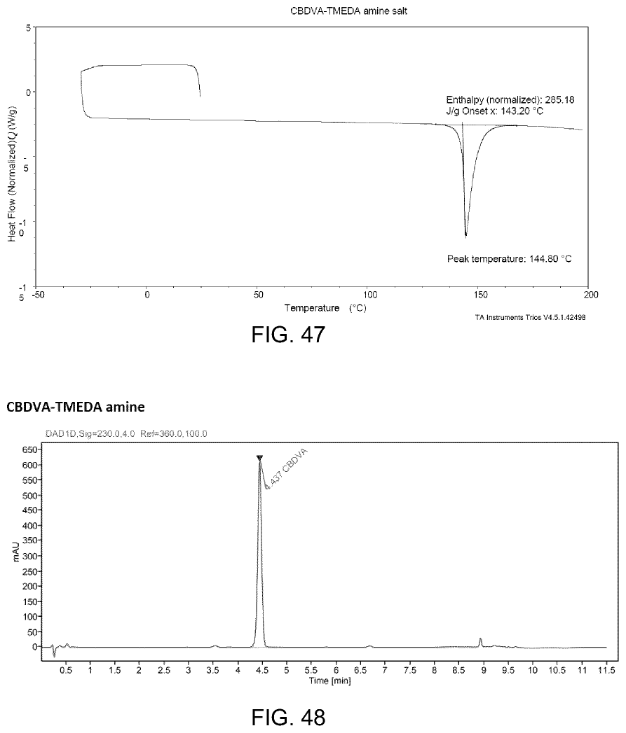

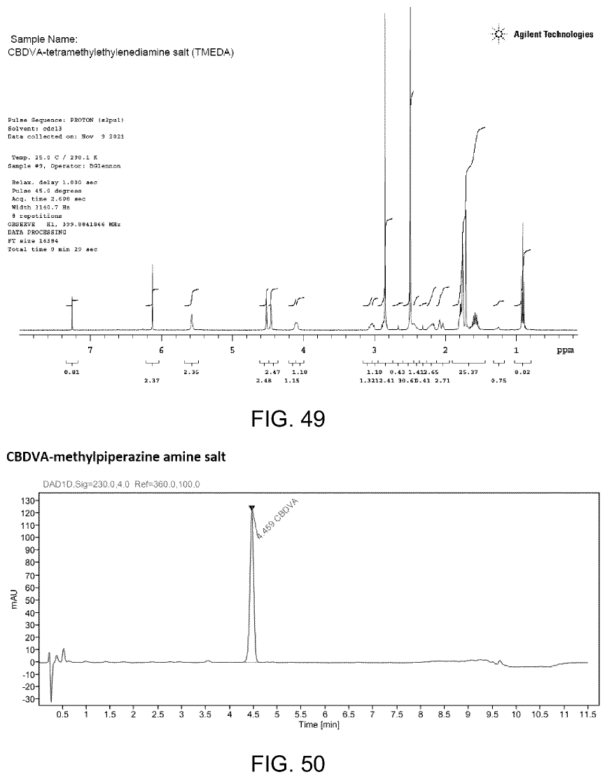

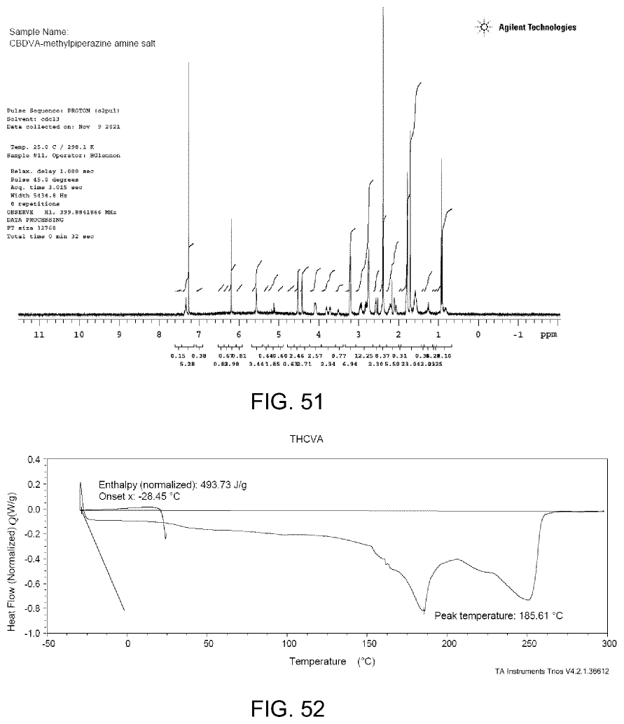

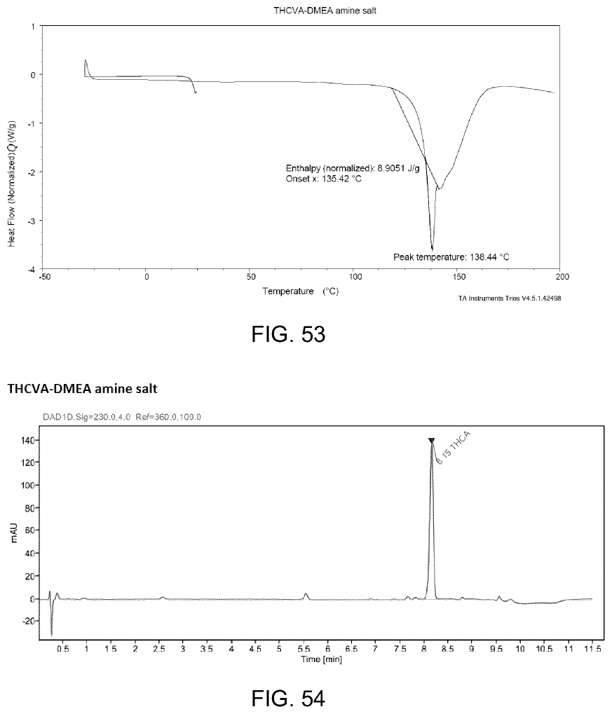

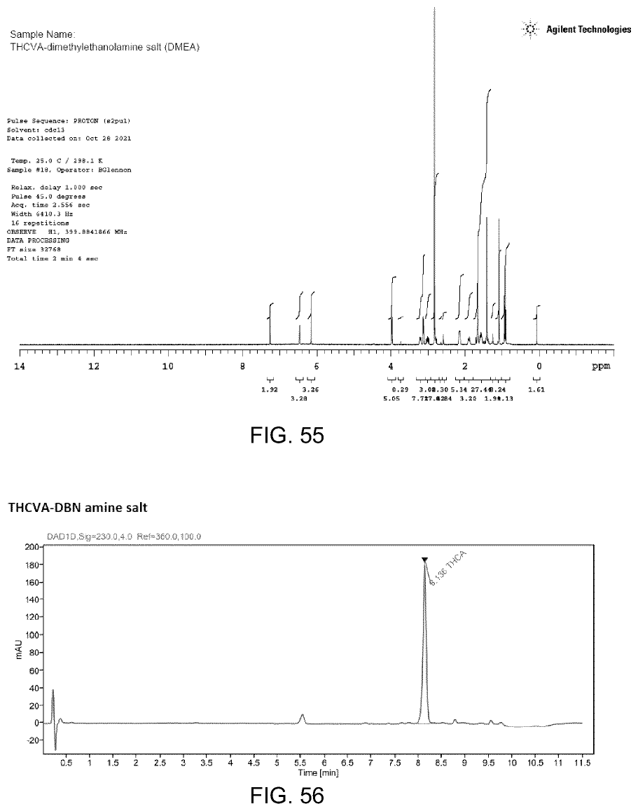

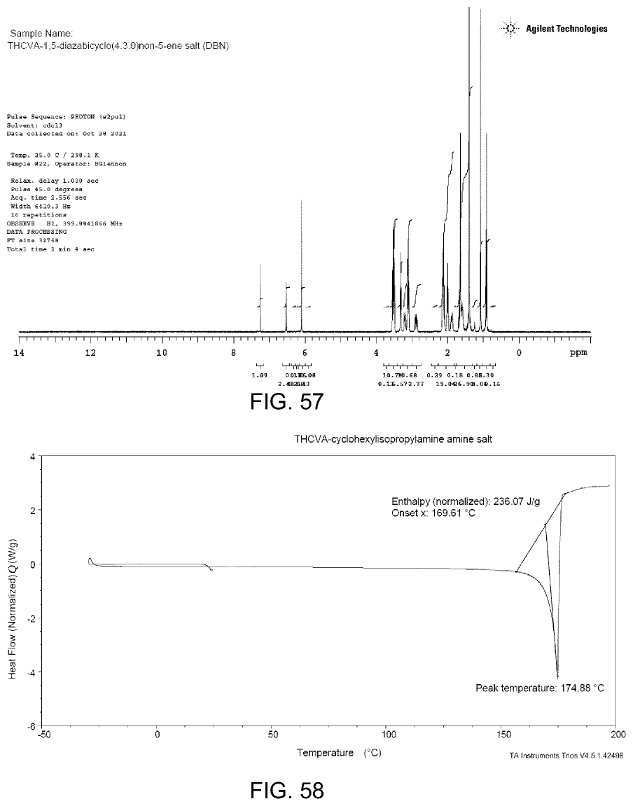

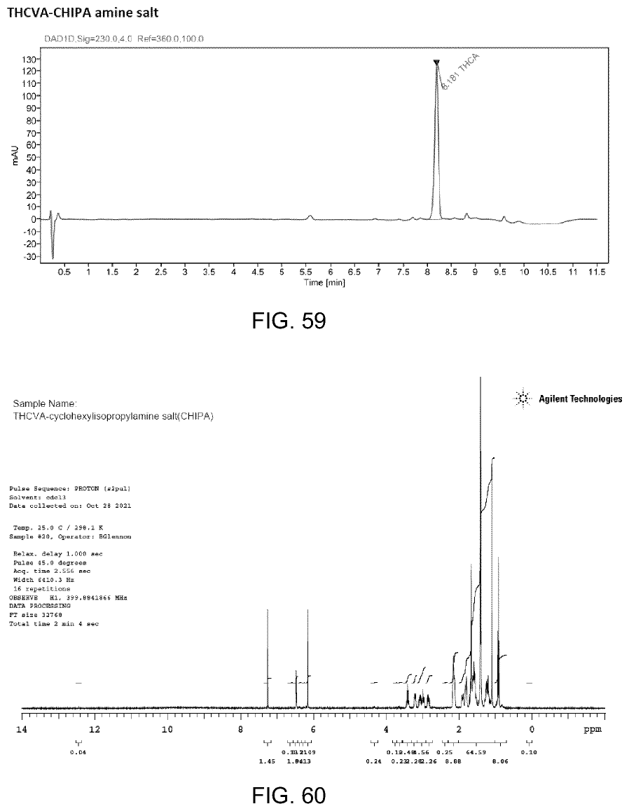

FIG. 20 is an HPLC chromatogram showing the cannabinoid content of a standardized crude extract produced from a dry powdered hemp kief sample and resolubilized in ethyl acetate in Example 8;

FIG. 21 is an HPLC chromatogram showing the cannabinoid content of the crude CBGA-Hunig's base amine salt precipitated from the standardized crude extract in Example 8, wherein the crude CBGA-amine salt was precipitated by addition of Hunig's base to the ethyl acetate-solubilized crude extract with a 1:1 vol./vol. heptane spike;

FIG. 22 is an HPLC chromatogram showing the cannabinoid content of the crude CBGA-Hunig's base amine salt precipitated from the standardized crude extract in Example 8, wherein the crude CBGA-amine salt was precipitated by addition of Hunig's base to the ethyl acetate-solubilized crude extract with a 1:1.75 vol./vol. heptane spike;

FIG. 23 is an HPLC chromatogram showing the cannabinoid content of the crude CBGA-Hunig's base amine salt precipitated from the standardized crude extract in Example 8, wherein the crude CBGA-amine salt was precipitated by addition of Hunig's base to the ethyl acetate-solubilized crude extract without a heptane spike;

FIG. 24 is an HPLC chromatogram showing the cannabinoid content of a standardized crude extract produced from a dry powdered hemp kief sample and resolubilized in 2-propanol in Example 9;

FIG. 25 is an HPLC chromatogram showing the cannabinoid content of the crude CBGA-Hunig's base amine salt precipitated from the standardized crude extract in Example 9, wherein the crude CBGA-amine salt was precipitated by addition of Hunig's base to the 2-propanol-solubilized crude extract with a 1:1 vol./vol. heptane spike;

FIG. 26 is an HPLC chromatogram showing the cannabinoid content of the crude CBGA-Hunig's base amine salt precipitated from the standardized crude extract in Example 9, wherein the crude CBGA-amine salt was precipitated by addition of Hunig's base to the 2-propanol-solubilized crude extract with a 1.75:1 vol./vol. heptane spike;

FIG. 27 is an HPLC chromatogram showing the cannabinoid content of a purified recrystallized CBGA-Hunig's base amine salt precipitated from a 2-propanol solution with a 1:1 vol./vol. heptane spike in Example 9;

FIG. 28 is an HPLC chromatogram showing the cannabinoid content of a purified recrystallized CBGA-Hunig's base amine salt precipitated from a 2-propanol solution without a heptane spike in Example 9;

FIG. 29 is an HPLC chromatogram showing the cannabinoid content of a standardized crude extract produced from a dry powdered hemp kief sample and resolubilized in 1-butanol in Example 10;

FIG. 30 is an HPLC chromatogram showing the cannabinoid content of the crude CBGA-Hunig's base amine salt precipitated from the standardized crude extract in Example 10, wherein the crude CBGA-amine salt was precipitated by addition of Hunig's base to the 1-butanol-solubilized crude extract with a 1:1 vol./vol. heptane spike;

FIG. 31 is an HPLC chromatogram showing the cannabinoid content of the crude CBGA-Hunig's base amine salt precipitated from the standardized crude extract in Example 10, wherein the crude CBGA-amine salt was precipitated by addition of Hunig's base to the 1-butanol-solubilized crude extract with a 1.75:1 vol./vol. heptane spike;

FIG. 32 is an HPLC chromatogram of a standards solution containing CBDVA, CBDA, CBGA, THCVA, and THCA for Examples 11 and 12;

FIG. 33 is a differential scanning calorimetry (DSC) thermogram produced for cannabidivarinic acid (CBDVA) in Example 11;

FIG. 34 is a DSC thermogram produced for a CBDVA-triethylamine salt (CBDVA-TEA);

FIG. 35, is an HPLC chromatogram of a CBDVA-TEA amine salt produced in Example 11;

FIG. 36 is chart showing a ¹H-NMR analysis of the CBDVA-triethylamine salt produced in Example 11;

FIG. 37 is a DSC thermogram produced for a CBDVA-N,N-diisopropylethylamine salt (Hunig's base) produced in Example 11;

FIG. 38 is an HPLC chromatogram of the CBDVA-Hunig's base amine salt produced in Example 11;

FIG. 39 is chart showing a ¹H-NMR analysis of the CBDVA-N,N-diisopropylethylamine salt produced in Example 11;

FIG. 40 is a DSC thermogram produced for a CBDVA-1,5-diazabicyclo(4.3.0)non-5-ene salt (DBN) produced in Example 11;

FIG. 41 is an HPLC chromatogram of the CBDVA-DBN amine salt produced in Example 11;

FIG. 42 is chart showing a ¹H-NMR analysis of the CBDVA-DBN salt produced in Example 11;

FIG. 43 is a DSC thermogram produced for a CBDVA-dimethylethanolamine salt (DMEA) produced in Example 11;

FIG. 44 is an HPLC chromatogram of the CBDVA-DMEA amine salt produced in Example 11;

FIG. 45 is chart showing a ¹H-NMR analysis of the CBDVA-DMEA salt produced in Example 11;

FIG. 46 is chart showing a ¹H-NMR analysis of the CBDVA-cyclohexylisopropylamine salt produced in Example 11;

FIG. 47 is a DSC thermogram produced for a CBDVA-tetramethylethylenediamine salt (TMEDA) produced in Example 11;

FIG. 48 is an HPLC chromatogram of the CBDVA-TMEDA amine salt produced in Example 11;

FIG. 49 is chart showing a ¹H-NMR analysis of the CBDVA-TMEDA salt produced in Example 11;

FIG. 50 is an HPLC chromatogram of a CBDVA-methylpiperazine amine salt produced in Example 11;

FIG. 51 is chart showing a ¹H-NMR analysis of a CBDVA-methylpiperazine salt produced in Example 11;

FIG. 52 is a DSC thermogram produced for Δ9-tetrahydrocannabivirinic acid (THCVA) produced in Example 12;

FIG. 53 is a DSC thermogram produced for a THCVA-dimethylethanolamine salt (DMEA) produced in Example 12;

FIG. 54 is an HPLC chromatogram of the THCVA-DMEA amine salt produced in Example 12;

FIG. 55 is a chart showing a ¹H-NMR analysis of the THCVA-DMEA salt produced in Example 12;

FIG. 56 is an HPLC chromatogram of the THCVA-1,5-diazabicyclo(4.3.0)non-5-ene salt (DBN) amine salt produced in Example 12;

FIG. 57 is a chart showing a ¹H-NMR analysis of the THCVA-DBN salt produced in Example 12;

FIG. 58 is a DSC thermogram produced for a THCVA-cyclohexylisopropylamine salt (CHIPA) produced in Example 12;

FIG. 59 is an HPLC chromatogram of the THCVA-CHIPA amine salt produced in Example 12; and

FIG. 60 is a chart showing a ¹H-NMR analysis of the THCVA-CH IPA salt produced in Example 12.

### DETAILED DESCRIPTION

No language or terminology in this specification should be construed as indicating any non-claimed element as essential or critical. All methods described herein can be performed in any suitable order unless otherwise indicated herein. The use of any and all examples, or example language (e.g., “such as”) provided herein, is intended merely to better illuminate example embodiments and does not pose a limitation on the scope of the claims appended hereto unless otherwise claimed.

It should be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

Throughout this specification, the word “comprise”, or variations such as “comprises”, “comprising”, “including”, “containing”, and the like, will be understood to imply the inclusion of a stated element or integer or group of elements or integers but not the exclusion of any other element or integer or group of elements or integers, unless the context requires otherwise.

To facilitate understanding of the embodiments set forth herein, a number of terms are defined below. Generally, the nomenclature used herein and the laboratory procedures in biology, biochemistry, organic chemistry, medicinal chemistry, pharmacology described herein are generally well known and commonly employed in the art. Unless defined otherwise, all technical and scientific terms used herein generally have the same meaning as commonly understood in the art to which this disclosure belongs. In the event that there is a plurality of definitions for a term used herein, those in this written description shall prevail unless stated otherwise herein.

As used herein, the singular forms “a”, “an”, and “the,” may also refer to plural articles, i.e., “one or more”, “at least one”, “and/or”, are open-ended expressions that are both conjunctive and disjunctive in operation. For example, the term “a cannabinoid” includes “one or more cannabinoids”. Further, each of the expressions “at least one of A, B, and C”, “at least one of A, B, or C”, “one or more of A, B, and C”, “one or more of A, B, or C” and “A, B, and/or C” means A alone, B alone, C alone. A and B together. A and C together, B and C together, or A, B and C together. The term “an entity” refers to one or more of that entity. As such, the terms “a”, “an”, “one or more”, and “at least one” can be used interchangeably herein.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller subranges are also included. The upper and lower limits of these smaller ranges are also included therein, subject to any specifically excluded limit in the stated range.

The terms “about” or “approximately” as used herein, mean an acceptable error for a particular recited value, which depends in part on how the value is measured or determined. In certain embodiments, “about” can mean one or more standard deviations. When the antecedent term “about” is applied to a recited range or value it denotes an approximation within the deviation in the range or value known or expected in the art from the measurement method. For removal of doubt, it shall be understood that any range stated in this written description that does not specifically recite the term “about” before the range or before any value within the stated range inherently includes such term to encompass the approximation within the deviation noted above.

As used herein, the terms “Cannabis” and “Cannabis biomass” encompass whole Cannabis sativa plants and also parts thereof which contain cannabinoids and Cannabis phytochemicals, such as the aerial parts of the plants or isolated leaves and/or flowering heads and/or seeds. The term also encompasses freshly harvested Cannabis plant material and also plant material, Cannabis plant material that was dried after harvesting. Dried Cannabis plant material may be in a loose form or alternatively, may be baled into square bales or rectangular bales or round bales or alternatively, may be compressed into cubes or pellets or cubes. Dried Cannabis plant material may be separated into two or more components wherein one component comprises the Cannabis stalks and stems, and a second component comprises the leaves, trichomes, and flowers. The second component may be further separated into leaves and trichome/flower components and the trichome/flower components may be separated into trichome and flower components. The term “kief” as used herein, refers to a powdery resin that is produced by and may be collected from the trichomes of Cannabis plants.

The separated dried Cannabis plant material components may be stored in a loose form and/or processed into a baled form and/or processed into a compressed form. The separated dried Cannabis plant material components may be packaged and stored in a packaging material.

Freshly harvested and/or dried harvested Cannabis biomass may be processed with a selected solvent to separate and recover therefrom in a crude extract, a complex mixture of cannabinoids and Cannabis phytochemicals.

The term “Cannabis phytochemicals” as used herein, refers to biologically active compounds produced by Cannabis sativa plants, and in particular, to mixtures of terpenes, terpenoids, flavonoids, alkaloids, lignans, omega fatty acids, pigments, and the like, that may be extracted and separated from Cannabis biomass by solvent extraction. The term “phytochemical” as used herein, refers to a single biologically active compound that has been separated from a complex mixture of phytochemicals obtained by solvent extraction of Cannabis biomass or from cultured microbial fermentation systems.

The term “cannabinoid” as used herein encompasses cannabidiol (CBD), cannabidiolic acid (CBDA), cannabinol (CBN), cannabigerol (CBG), cannabigerolic acid (CBGA), cannabichromene (CBC), cannabichromenic (CBCA), cannabicyclol (CBL), cannabivarin (CBV), cannabidivarin (CBDV), cannabidivarinic acid (CBDVA), cannabichromevarin (CBCV), cannabigerovarin (CBGV), cannabigerol monomethyl ether (CBGM), cannabielsoin (CBE), cannabicitran (CBT), among others. The term “cannabinoid” may also be substituted for herein by the acronym “CBD”. The term “tetrahydrocannabinol” as used herein encompasses (-)-trans-Δ9-tetrahydrocannabinol (Δ9-THC). Δ8-tetrahydrocannabinol (Δ8-THC), iso-tetrahydrocannabinol, tetrahydrocannabinolic acid (THCA), tetrahydrocannabivarin (THCV), tetrahydrocannabivarinic acid (THCVA), among others. The term “tetrahydrocannabinol” may also be substituted for herein by the acronym “THC”.

It is to be noted that fermentation broths wherein genetically modified microorganisms have been cultured to produce target cannabinoid phytochemicals, may be processed to separate and recover therefrom complex mixtures of fermentation metabolites and phytochemicals. The processing may include separating the cultured microorganisms from the fermentation broths prior to separation and recovery of complex mixtures of fermentation metabolites and phytochemicals. The processing may include steps to recover metabolites and phytochemicals from the cultured microorganisms. The complex mixtures may be processed by one or more of dewatering steps, desolventizing steps, drying steps, filtration steps including microfiltration steps, extraction with supercritical CO₂ steps, and the like known to those skilled in this art, to produce a concentrated crude complex mixture in an oil form or a solid form or a dry form. Suitable methods for preparing complex mixtures of fermentation metabolites and phytochemicals from fermentation broths and from cultured microorganism may be found in International Patent Application Publication No. WO 2020/160284A1, International Patent Application Publication No. WO 2020/176998CA, IUS Patent Application Publication No. 2021/0189444A1, Chapter 10 “The recovery and purification of fermentation products” in the third edition of “Principles of Fermentation Technology” (Stanbury et al., 2016).

The term “solvent” as used herein, is used herein to denote a liquid or gas capable of dissolving a solid or another liquid or gas. Non-limiting examples of solvents include alcohols such as methanol, ethanol, propanol, isopropanol, butanol, alkanes such as propane, butane, hexane, heptane, pentane, and the like, ethyl acetate, acetone (also known as propanone), dichloromethane, 1,4-dioxane, tetrahydrofuran, acetonitrile, toluene, methyl tert-butyl ether, supercritical carbon dioxide (CO₂), subcritical CO₂, and the like.

The term “solvent switching” as used herein refers to a process for extracting, separating, recovering, and purifying selected cannabinoids from Cannabis biomass wherein the first step is to process a Cannabis biomass with a first solvent selected from methanol, ethanol, propanol, isopropanol, butanol, propane, butane, ethyl acetate, acetone, dichloromethane, 1,4-dioxane, tetrahydrofuran, acetonitrile, toluene, methyl tert-butyl ether, supercritical carbon dioxide (CO₂), subcritical CO₂, and the like, to produce a crude Cannabis extract. The next step is to desolventize the crude Cannabis extract to recover the extractant solvent therefrom to produce a concentrated Cannabis extract in the form of an oil or a resin or a solid. The next step is to resolubilize the concentrated Cannabis extract into a second solvent (that is, switching the processing solvents) selected from one of ethyl acetate, methanol, ethanol, isopropanol, propanol, butanol, dichloromethane, C5-C7 low-boiling hydrocarbon solvents including alkanes and petroleum ethers to thereby produce a solvent-switched crude Cannabis extract.

As used herein, the term “antisolvent” refers to an organic solvent that may be used to precipitate a target compound or molecule from another solvent in which the target compound or molecule is completely dissolved whereby, as the antisolvent is added to the solvent containing the dissolved target compound or molecule, the precipitation process is initiated by nucleation of the target compound or molecule followed by the formation of solid particles. When an alcohol was a solvent selected for dissolution of a target compound or molecule, water may be a suitable antisolvent to precipitate the target compound or molecule.

The term “crude precipitate” as used herein means the solids and/or oils produced by a chemical reaction between a selected organic base with a mixture of cannabinoid carboxylic acids present in a crude Cannabis extract. The “crude precipitate” may also be referred to herein as a “crude isolate” or a “carboxylic acid salt” or a “precipitated cannabinoid”.

The term “purified precipitate” as used herein means the solids and/or oils remaining after the crude precipitate is washed with a selected solvent such as, for example, with ethyl acetate. A purified precipitate may also be produced via a recrystallization process wherein the crude precipitate is dissolved in a heated solvent and then cooled to an appropriate temperature to induce crystallization. Alternatively, the crude precipitate may be dissolved in a solvent which readily dissolves both the desired purified precipitate and the impurities present in the crude precipitate, followed by addition of an antisolvent in which the desired precipitate is insoluble and the impurities remain in solution. Subsequent filtration yields the purified precipitate. The “purified precipitate” may also be referred as a “purified isolate” or a “purified cannabinoid precipitate” or a “purified cannabinoid carboxylic acid”.

As used herein, the term a “standardized solvent-solubilized complex extract or mixture” refers to a complex extract or complex mixture that has been adjusted by the addition or removal of a solvent to adjust the concentrations therein of one or more bioactive markers, such as CBGA or THCVA or CBGVA or CBCVA to a selected target range in comparison to the concentrations of the one or more bioactive markers in a reference solution, using analytical methods known to those skilled in these arts. For example, suitable analytical methods include HPLC methods and the like.

Some embodiments disclosed herein relate to methods of separating and recovering CBGA or THCVA or CBGVA or CBCVA or CBCA from solvent-solubilized complex extracts or mixtures comprising cannabinoids and other phytochemicals extracted and recovered from Cannabis biomass feedstocks or from cultured microbial fermentation systems. The methods for specifically separating and recovering CBGA and/or THCVA and/or CBDVA and/or CBCVA and/or CBCA from solvent-solubilized complex extracts or mixtures pertain to the use of one or more selected amines to react with CBGA and/or THCVA and/or CBDVA and/or CBCVA and/or CBCA thereby forming CBGA-amine salts and/or THCVA-amine salts and/or CBDVA-amine salts and/or CBCVA-amine salts and/or CBCA-amine salts that precipitate out of the solvent-solubilized complex extracts and mixtures. The methods disclosed herein include steps for separating and recovering precipitated CBGA-amine salts and/or THCVA-amine salts and/or CBDVA-amine salts and/or CBCVA-amine salts and/or CBCA-amine salts from solvent-solubilized complex extracts or mixtures, for washing recovered CBGA-amine salts and/or THCVA-amine salts and/or CBDVA-amine salts to and/or CBCVA-amine salts and/or CBCA-amine salts to separate and remove therefrom other cannabinoids and Cannabis phytochemicals that may have been recovered with the precipitated CBGA-amine salts and/or THCVA-amine salts and/or CBDVA-amine salts and/or CBCVA-amine salts and/or CBCA-amine salts, for further purifying and recrystallization of the washed CBGA-amine salts and/or THCVA-amine salts and/or CBDVA-amine salts and/or CBCVA-amine salts and/or CBCA-amine salts, for the preparation of purified crystalline CBGA and/or THCVA and/or CBDVA, and/or CBCVA and/or CBCA, and for decarboxylating the purified CBGA-amine salts and/or THCVA-amine salts and/or CBDVA-amine salts and/or CBCVA-amine salts and/or CBCA-amine salts to produce purified CBG and/or THCV and/or CBDV and/or CBCV and/or CBC therefrom.

Without being bound by any theory of operation or mechanism of action, the examples of embodiments disclosed herein are based in part, on an unpredicted/unexpected discovery that use of an amine having a suitably placed heteroatom can effectuate the transfer of the acidic proton from the carboxylic acid to the amine by stable/strong hydrogen bonding in the ammonium ion, as shown below, and thereby drive the acid-base reaction to completion by facilitating the crystallization of the desired salt as shown in Eqn 1 and Eqn 2:

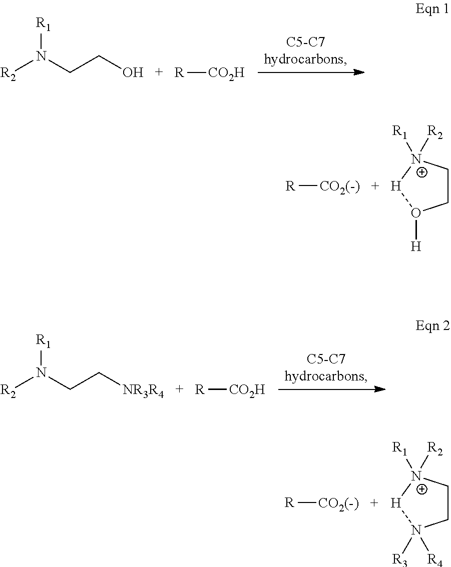

It was surprisingly discovered that some amines precipitated CBGA salts from desolventized crude Cannabis extracts that were resolubilized in certain organic solvents selected from ethyl acetate, alcohols, dichloromethane (DCM), acetone, and mixtures thereof. It was also discovered that some amines precipitated CBGA salts from desolventized crude Cannabis extracts that were resolubilized in methanol solvents after the addition of water to the amine-desolventized crude Cannabis extract mixtures. The amine-precipitated CBGA salts, also referred to herein as CBGA-amine salts, have low or no solubility in a number of organic solvents at room temperature and therefore, may be washed with those organic solvents to produce highly purified CBGA-amine salts.

Some amines may precipitate CBDVA salts from desolventized complex extracts and mixtures that were resolubilized in certain organic solvents selected from ethyl acetate, isopropanol, propanol, butanol, and acetone. Some amines may precipitate CBDVA salts from desolventized complex extracts and mixtures that were resolubilized in ethanol solvents after the addition of water to the amine-desolventized crude Cannabis extract mixtures. The amine-precipitated CBDVA salts, also referred to herein as CBDVA-amine salts, have low or no solubility in a number of organic solvents at room temperature and therefore, may be washed with those organic solvents to produce highly purified CBDVA-amine salts.

Some amines may precipitate THCVA salts from desolventized complex extracts and mixtures that were resolubilized in certain organic solvents selected from ethyl acetate, ethanol, isopropanol, propanol, butanol, and acetone. Some amines may precipitate THCVA salts from desolventized complex extracts and mixtures that were resolubilized in ethanol solvents after the addition of water to the amine-desolventized complex extracts and mixtures. The amine-precipitated THCVA salts, also referred to herein as THCVA-amine salts, have low or no solubility in a number of organic solvents at room temperature and therefore, may be washed with those organic solvents to produce highly purified THCVA-amine salts.

Some amines may precipitate CBCVA salts from desolventized complex extracts and mixtures that were resolubilized in certain organic solvents selected from ethyl acetate, ethanol, isopropanol, propanol, butanol, acetone, dichloromethane and the like. Some amines may precipitate CBCVA salts from desolventized complex extracts mixtures that were resolubilized in ethanol solvents after the addition of water to the amine-desolventized crude Cannabis extract mixtures. The amine-precipitated CBCVA salts, also referred to herein as CBCVA-amine salts, have low or no solubility in a number of organic solvents at room temperature and therefore, may be washed with those organic solvents to produce highly purified CBCVA-amine salts.

Some amines may precipitate CBCA salts from desolventized complex extracts and mixtures that were resolubilized in certain organic solvents selected from ethyl acetate, ethanol, isopropanol, propanol, butanol, and acetone. Some amines may precipitated CBCA salts from desolventized complex extracts mixtures that were resolubilized in ethanol solvents after the addition of water to the amine-desolventized crude Cannabis extract mixtures. The amine-precipitated CBCA salts, also referred to herein as CBCA-amine salts, have low or no solubility in a number of organic solvents at room temperature and therefore, may be washed with those organic solvents to produce highly purified CBCA-amine salts.

According to one embodiment of the present disclosure, it was discovered that addition at room temperature of certain tertiary amines such as triethylamine, tripropylamine, tributylamine, N,N-diisopropylethylamine (Hunig's base), and methyldicyclohexylamine to solvent-solubilized complex extracts and mixtures comprising complex mixtures of metabolites, cannabinoids and Cannabis phytochemicals, precipitated CBGA-amine salts, from the crude extracts. It was also discovered that certain diamines such as 1,4-diazabicyclo[2.2.2]octane (DABCO) precipitated CBGA-amine salts from solvent-solubilized complex extract and mixtures. It was also discovered that certain secondary amines such as dicyclohexylamine, isopropylcyclohexylamine, and 2,2,6,6-tertamethylpiperidine precipitated CBGA-amine salts from solvent-solubilized complex extracts and mixtures.

According to another embodiment of the present disclosure, addition at room temperature of certain tertiary amines such as triethylamine and N,N-diisopropylethylamine (Hunig's base) to solvent-solubilized complex extracts and mixtures comprising complex mixtures of metabolites, cannabinoids and Cannabis phytochemicals, precipitated CBDVA-amine salts, from the crude extracts. It was also discovered that certain amino alcohols such as dimethylethanolamine (DMEA) precipitated CBDVA-amine salts from solvent-solubilized complex extract and mixtures. It was also discovered that certain highly basic amines such as 1,5-diazabicyclo(4.3.0)non-5-ene (DBN) precipitated CBDVA-amine salts from solvent-solubilized complex extract and mixtures. It was also discovered that certain diamines such as 1,4-diazabicyclo[2.2.2]octane (DABCO) and N-methylpiperazine precipitated CBDVA-amine salts from solvent-solubilized complex extract and mixtures at temperatures of about 0° C. It was also discovered that N-cyclohexylisopropylamine precipitated CBDVA-amine salts from solvent-solubilized complex extract and mixtures at temperatures of about 0° C.

According to another embodiment of the present disclosure, addition at about 0° C. of dimethylethanolamine (DMEA) or 1,5-diazabicyclo(4.3.0)non-5-ene (DBN) or N-cyclohexylisopropylamine to solvent-solubilized complex extracts and mixtures comprising complex mixtures of metabolites, cannabinoids and Cannabis phytochemicals, precipitated THCVA-amine salts, from the crude extracts.

According to another embodiment of the present disclosure, addition at room temperature of isopropylcyclohexylamine or 2,2,6,6-tetramethylpiperidine or dicyclohexylamine or N,N-diisopropylethylamine (Hunig's base) or 1,5-diazabicyclo[4.3.0]non-5-ene (DBN) or 1,4-diazabicyclo[2.2.2]octane (DABCO) or dimethylaminopyridine (DMAP) to solvent-solubilized complex extracts and mixtures comprising complex mixtures of metabolites, cannabinoids and Cannabis phytochemicals, precipitated CBCVA-amine salts, from the crude extracts and mixtures.

According to another embodiment of the present disclosure, that addition at room temperature of 1,5-diazabicyclo[4.3.0]non-5-ene (DBN) of 4-dimethylaminopyridine to solvent-solubilized complex extracts and mixtures comprising complex mixtures of metabolites, cannabinoids and Cannabis phytochemicals, precipitated CBCA-amine salts from the crude extracts and mixtures.

According to another embodiment of the present disclosure, CBGA-amine salts, THCVA-amine salts, CBDVA-amine salts, CBCVA-amine salts, CBCA-amine salts that formed an oil and/or precipitated as a solids salt by a selected amine as disclosed herein, may be washed with a selected solvent to remove other cannabinoids and/or Cannabis phytochemicals that may have remained associated with the recovered precipitated CBGA-amine salts, THCVA-amine salts, CBDVA-amine salts, CBCVA-amine salts, CBCA-amine salts. Suitable solvents for the washing step include ethyl acetate, ethanol, isopropanol, propanol, butanol, dichloromethane, heptane, hexane, and the like.

According to another embodiment of the present disclosure, washed CBGA-amine salts, THCVA-amine salts, CBDVA-amine salts, CBCVA-amine salts, and CBCA-amine salts may be further purified by addition and mixing into a heated mixture of a solvent pair to form a solution, and then, may be recrystallized back into purified CBGA-amine salts, THCVA-amine salts, CBDVA-amine salts, CBCVA-amine salts and CBCA-amine salts by cooling or by the addition of an antisolvent. According to an aspect, a suitable solvent may be one of ethyl acetate, 95% ethanol, denatured ethanol, methanol, isopropanol, dichloromethane, toluene, methyl-tert-butyl ether (MTBE), tetrahydrofuran (THF), and the like. A particularly suitable solvent pair is a mixture of ethyl acetate with heptane. Suitable antisolvents for use with such solvents include C5-C7 alkanes and low b.p. petroleum ethers. A particularly suitable antisolvent may be heptane.

According to another aspect, a suitable ratio for the solvent pair mixture may be selected from a range of about 5:1 to about 20:1. A particularly suitable solvent pair ratio may be about 10:1, for example 10 parts ethyl acetate and 1 part heptane.

The CBGA-amine salts/polar solvent/non-polar solvent solution is then cooled to about 30° C., and then may be placed into a 4° C. environment for a period of time selected from about 30 min to about 12 h during which time, purified CBGA-amine salt will recrystallize out of the polar solvent/non-polar solvent mixture. The recrystallized purified CBGA-amine salt may then be separated from the polar solvent/non-polar solvent mixture, for example, by filtration or centrifugation.

The THCVA-amine salts/polar solvent/non-polar solvent solution is then cooled to about 30° C., and then may be placed into a 4° C. environment for a period of time selected from about 30 min to about 12 h during which time, purified THCVA-amine salt will recrystallize out of the polar solvent/non-polar solvent mixture. The recrystallized purified THCVA-amine salt may then be separated from the polar solvent/non-polar solvent mixture, for example, by filtration or centrifugation.

The CBDVA-amine salts/polar solvent/non-polar solvent solution is then cooled to about 30° C., and then may be placed into a 4° C. environment for a period of time selected from about 30 min to about 12 h during which time, purified CBDVA-amine salt will recrystallize out of the polar solvent/non-polar solvent mixture. The recrystallized purified CBDVA-amine salt may then be separated from the polar solvent/non-polar solvent mixture, for example, by filtration or centrifugation.

The CBCVA-amine salts/polar solvent/non-polar solvent solution is then cooled to about 30° C., and then may be placed into a 4° C. environment for a period of time selected from about 30 min to about 12 h during which time, purified CBCVA-amine salt will recrystallize out of the polar solvent/non-polar solvent mixture. The recrystallized purified CBCVA-amine salt may then be separated from the polar solvent/non-polar solvent mixture, for example, by filtration or centrifugation.

The CBCA-amine salts/polar solvent/non-polar solvent solution is then cooled to about 30° C., and then may be placed into a 4° C. environment for a period of time selected from about 30 min to about 12 h during which time, purified CBCA-amine salt will recrystallize out of the polar solvent/non-polar solvent mixture. The recrystallized purified CBCA-amine salt may then be separated from the polar solvent/non-polar solvent mixture, for example, by filtration or centrifugation.

According to another embodiment of the present disclosure, purified CBGA-amine salts, THCVA-amine salts CBDVA-amine salts, CBCVA-amine salts, and CBCA-amine salts produced by the methods disclosed herein, may be decarboxylated and then separated from the amine moieties by acidification to thereby produce a purified CBG or a purified THCV or a purified CBDV or a purified CBCV or a purified CBC.

CBGA-amine salts, THCVA-amine salts, CBDVA-amine salts, CBCVA-amine salts, CBCA-amine salts produced by the methods disclosed herein, may be acidified to separate the amines therefrom to produce highly purified CBGA, THCVA, CBDVA, CBCVA, or CBCA.

Some embodiments of the present disclosure relate to purified CBGA-amine salts, THCVA-amine salts, CBDVA-amine salts, CBCVA-amine salts, and/or CBCA-amine salts that have been precipitated and recovered from a crude complex extract comprising a mixture of metabolites, cannabinoids, and Cannabis phytochemicals recovered from Cannabis biomass, or from a complex mixture of metabolites, cannabinoids, and Cannabis phytochemicals recovered from cultured microbial fermentation systems, with a suitable selected amine. An example method for producing purified CBGA-amine salts or THCVA-amine salts or CBDVA-amine salts or CBCVA-amine salts or CBCA-amine salts comprises the steps of:
1. providing a crude complex extract comprising a mixture of metabolites, cannabinoids, and Cannabis phytochemicals recovered from Cannabis biomass, or a crude complex mixture of metabolites, cannabinoids, and Cannabis phytochemicals recovered from cultured microbial fermentation systems;
2. assaying the crude complex extract or complex mixture to determine the content of CBGA or THCVA or CBDVA or CBCVA or CBCA therein;
3. adding a selected volume of a first organic solvent to the crude complex extract or complex mixture to thereby adjust the CBGA or THCVA or CBDVA or CBCVA or CBCA content therein to within a selected range in reference to a CBGA or THCVA or CBDVA or CBCVA or CBCA standard, thereby producing a standardized solvent-solubilized crude extract;
4. adding and mixing into the standardized solvent-solubilized crude extract, a selected volume of a selected amine whereby the ammonium moiety of the amine reacts with CBGA or THCVA or CBDVA or CBCVA or CBCA therein, thereby forming and precipitating a crude CBGA-amine salt and/or THCVA-amine salt and/or CBDVA-amine salt and/or CBCVA-amine salt and/or CBCA-amine salt;
5. separating and recovering the precipitated crude CBGA-amine salt or THCVA-amine salt or CBDVA-amine salt or CBCVA-amine salt or CBCA-amine salt from the standardized solvent-solubilized crude extract;
6. washing the recovered crude CBGA-amine salt or THCVA-amine salt or CBDVA-amine salt or CBCVA-amine salt or CBCA-amine salt with a selected second organic solvent one or more times to thereby produce a washed CBGA-amine salt or THCVA-amine salt or CBDVA-amine salt or CBCVA-amine salt or CBCA-amine salt;
7. re-solubilizing the washed CBGA-amine salt or THCVA-amine salt or CBDVA-amine salt or CBCVA-amine salt or CBCA-amine salt in a selected third organic solvent;
8 crystalizing the solubilized CBGA-amine salt or THCVA-amine salt or CBDVA-amine salt or CBCVA-amine salt or CBCA-amine salt by cooling and optionally, by the addition of and mixing with a selected antisolvent, to thereby produce a crystallized purified CBGA-amine salt or THCVA-amine salt or CBDVA-amine salt or CBCVA-amine salt or CBCA-amine salt;
9. separating, recovering, and washing the recrystallized purified CBGA-amine salt or THCVA-amine salt or CBDVA-amine salt or CBCVA-amine salt or CBCA-amine salt with the second organic solvent, then drying the purified CBGA-amine salt or THCVA-amine salt or CBDVA-amine salt or CBCVA-amine salt or CBCA-amine salt.

According to an aspect, a suitable first organic solvent for use in step 3 may be one of ethyl acetate, ethanol, denatured ethanol, isopropanol, propanol, butanol, and dichloromethane. According to another aspect, a suitable first solvent for use in step 3 may be a mixture of ethanol and a selected alkane, for example a 75:25 mixture of ethanol and heptane or a 75:25 mixture of ethanol and hexane.

According to an aspect, a suitable target range for adjusting the CBGA or THCVA or CBDVA or CBCVA or CBCA content in step 3 may be from about 20 mg/mL to about 445 mg/mL. A particularly suitable target range may be from about 27 mg/mL to about 200 mg/mL. A preferred target range may be from about 31 mg/mL to about 153 mg/mL.

According to another aspect, a suitable amine for use in step 4 for CBCA may be a tertiary amine such as triethylamine, tributylamine, N,N-diisopropylethylamine (Hunig's base), and methyldicyclohexylamine, or a diamine such as 1,4-diazabicyclo[2.2.2]octane (DABCO), 1,5-diazabicyclo[4.3.0]non-5-ene (DBN), or a secondary amines such as dicyclohexylamine, isopropylcyclohexylamine, and 2,2,6,6-tertamethylpiperidine.

According to another aspect, a suitable amine for use in step 4 for CBDVA at room temperature may be a tertiary amine such as triethylamine and N,N-diisopropylethylamine (Hunig's base), or an amino alcohol such as dimethylethanolamine (DMEA), a highly basic amine such as 1,5-diazabicyclo(4.3.0)non-5-ene (DBN). If so desired to perform step 3 for CBDVA at about 0° C., a suitable amine would be a diamine such as 1,4-diazabicyclo[2.2.2]octane (DABCO), N-methylpiperazine, N-cyclohexylisopropylamine, and tetramethylethylenediamine.

According to another aspect, a suitable amine for use in step 4 for THCVA at about 0° C. is dimethylethanolamine (DMEA) or 1,5-diazabicyclo(4.3.0)non-5-ene (DBN) or N-cyclohexylisopropylamine.

According to another aspect, a suitable amine for use in step 4 for CBCVA is 2,2,6,6-tetramethylpiperidine or N,N-diisopropylethylamine (Hunig's base) or 1,5-diazo-2,2,2-bicyclooctane (DBN) to solvent-solubilized complex extracts and mixtures comprising complex mixtures of metabolites, cannabinoids and Cannabis phytochemicals, from the crude extracts and mixtures.

According to another aspect, a suitable amine for use in step 4 for CBCA at room temperature of 2,2,6,6-tetramethylpiperidine or N,N-diisopropylethylamine (Hunig's base) or 1,4-diazabicyclo[2.2.2]octane (DABCO) to solvent-solubilized complex extracts and mixtures comprising complex mixtures of metabolites, cannabinoids and Cannabis phytochemicals, from the crude extracts and mixtures.

According to another aspect, a suitable second organic solvent for washing the crude CBGA-amine salt or CBCVA-amine salt or CBDVA-amine salt or THCVA-amine salt or CBCA-amine salt in step 6, may be a C5 to C7 alkane.

According to another aspect, a suitable third organic solvent for resolubilizing the washed crude CBGA-amine salt or CBCVA-amine salt or CBDVA-amine salt or THCVA-amine salt or CBCA-amine salt in step 7, may be one of ethyl acetate, 85%-99% ethanol, denatured ethanol, methanol, isopropanol, dichloromethane, toluene, MTBE, THF, and the like. A particularly suitable solvent for resolubilizing the washed CBGA-amine salt or CBCVA-amine salt or CBDVA-amine salt or THCVA-amine salt or CBCA-amine salt in step 6, may be ethyl acetate heated to about 60° C. or ethanol heated to about 40° C.

According to another aspect, a suitable antisolvent for recrystallizing the solubilized CBGA-amine salt or CBCVA-amine salt or CBDVA-amine salt or THCVA-amine salt or CBCA-amine salt in step 8, may be an alkane such as one of heptane, hexane, pentane, and the like. In the case wherein an alcohol is the selected third organic solvent, a suitable antisolvent may be heptane.

Another embodiment of the present disclosure pertains to an example method for separating out, recovering, and purifying one or more of CBGA, THCVA, CBDVA, CBCVA, and/or CBCA from a CBGA-amine salt, a THCVA-amine salt, a CBDVA-amine salt, a CBCVA-amine salt, and/or a CBCA-amine salt produced by following the method steps 1 to 9 previously disclosed herein, additionally comprising the steps of:
10. re-solubilizing the purified CBGA-amine salt or THCVA-amine salt or CBDVA-amine salt or CBCVA-amine salt or CBCA-amine salt in a selected fourth organic solvent, then
11. acidifying the solubilized purified CBGA-amine salt or THCVA-amine salt or CBDVA-amine salt or a CBCVA-amine salt or CBCA-amine salt with a mineral acid solution to partition therefrom an organic layer containing the highly purified CBGA or THCVA or CBDVA or CBCVA or CBCA, and an aqueous layer containing the amine as its hydrochloride;
12. separating the aqueous layer from the organic layer containing the highly purified CBGA or THCVA or CBDVA or CBCVA or CBCA; and
13. desolventizing the CBGA or THCVA or CBDVA or CBCVA or CBCA to produce highly purified CBGA or THCVA or CBDVA or CBCVA or CBCA.

According to an aspect, a suitable fourth organic solvent for use in step 10 may be one of ethyl acetate, ethanol, denatured ethanol, isopropanol, propanol, butanol, and dichloromethane.

According to another aspect, a suitable mineral acid solution for use in step 11 may be one of 5% HCl, 5% H₂SO₄, and the like.

Another embodiment of the present disclosure pertains to an example method for separating out, recovering, and purifying one or more of CBG, THCV, CBDV, CBCV, and/or CBC from a CBGA-amine salt, a THCVA-amine salt, a CBDVA-amine salt, a CBCVA-amine salt, and/or a CBCA-amine salt produced by following method steps 1 to 9 previously disclosed herein, additionally comprising the steps of:
14. decarboxylating the purified CBGA-amine salt or THCVA-amine salt or CBDVA-amine salt or CBCVA-amine salt or CBCA-amine salt to produce an oil containing CBG or CBCV or CBDV or THCV or CBC and amine;
15. solubilizing the oil containing decarboxylated CBG or CBCV or CBDV or THCV or CBC and amine in a selected fifth organic solvent to thereby partition therefrom an organic layer containing a highly purified CBG or CBCV or CBDV or THCV or CBC oil and separated amine organic layer, and an aqueous layer;
16. separating the organic layer containing the highly purified CBG or CBCV or CBDV or THCV or CBC and amine from the aqueous layer;
17. acidifying the organic layer with a mineral acid solution to partition therefrom an organic layer containing the highly purified CBG or CBCV or CBDV or THCV or CBC in the form of an oil, and an aqueous layer containing the amine as its hydrochloride;
18. dewatering and drying the purified CBG or THCV or CBDVA or CBCVA or CBCA organic layer; and
19. removing the fifth organic solvent from the highly purified CBG or CBCV or CBDV or THCV or CBC.

According to another aspect, the recrystallized purified CBGA-amine salt or CBCVA-amine salt or CBDVA-amine salt or THCVA-amine salt or CBCA-amine salt may be decarboxylated in step 14, by adding the CBCA-amine salt or CBCVA-amine salt or CBDVA-amine salt or THCVA-amine salt or CBCA-amine salt into a sodium carbonate (Na₂CO₃) solution, then heating the mixture under constant mixing at a temperature selected from a range of about 98-102° C. to reflux for a period of time selected from a range of about 2 hr to about 18 hr, thereby producing a biphasic solution of CBG or CBCV or CBDV or THCV or CBC oil and separated amine organic phase, and an aqueous phase containing the Na₂CO₃ solution. A suitable concentration of Na₂CO₃ solution to use for this step is from a range of about 1% to about 15% (w/v). A particularly suitable concentration of Na₂CO₃ solution is from a range of about 2.5% to about 10% (w/v), for example, about 5% (w/v). A particularly suitable temperature for this decarboxylation step is about 100° C. A particularly suitable time duration for this decarboxylation step is about 4 hr.

According to an aspect, a suitable fifth organic solvent for use in step 15 may be one of dichloromethane or C5 to C7 alkane and the like.

According to another aspect, a suitable mineral acid solution for use in step 17 may a 5% HCl solution, a 5% H₂SO₄ solution, and the like.

The following examples are provided to more fully describe the invention and are presented for non-limiting illustrative purposes.

### EXAMPLES

### Example 1

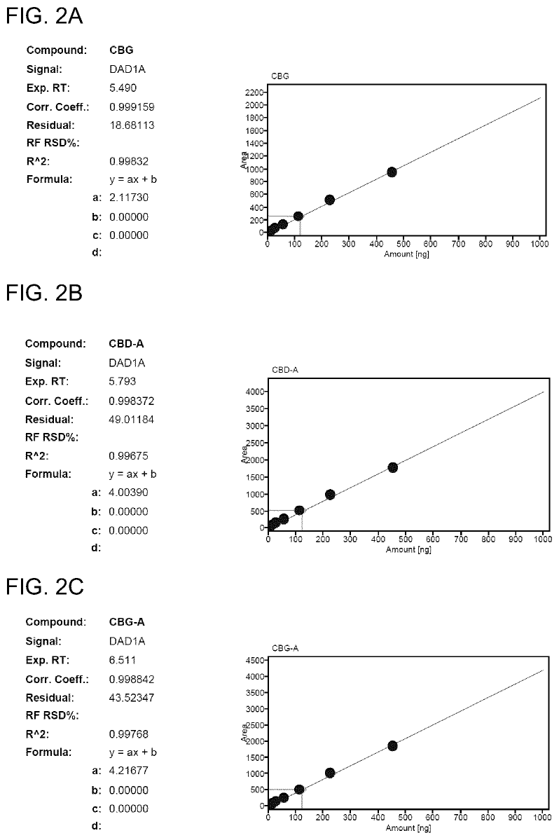

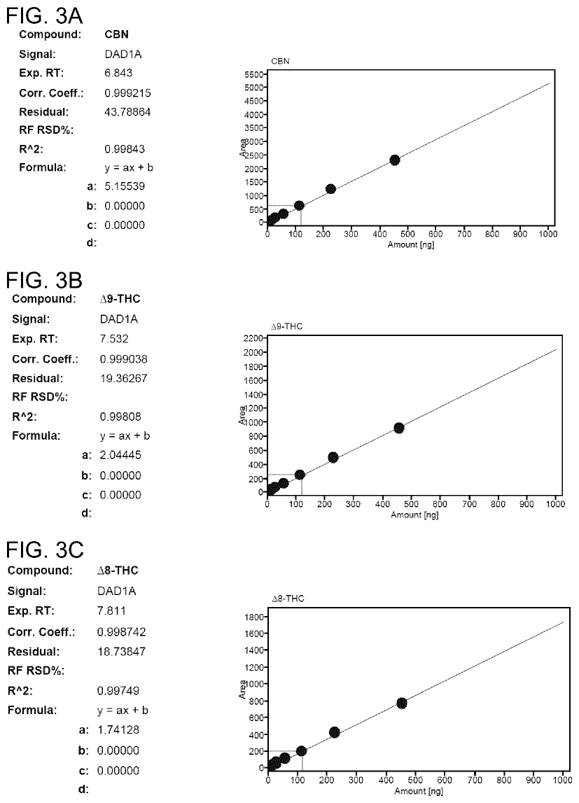

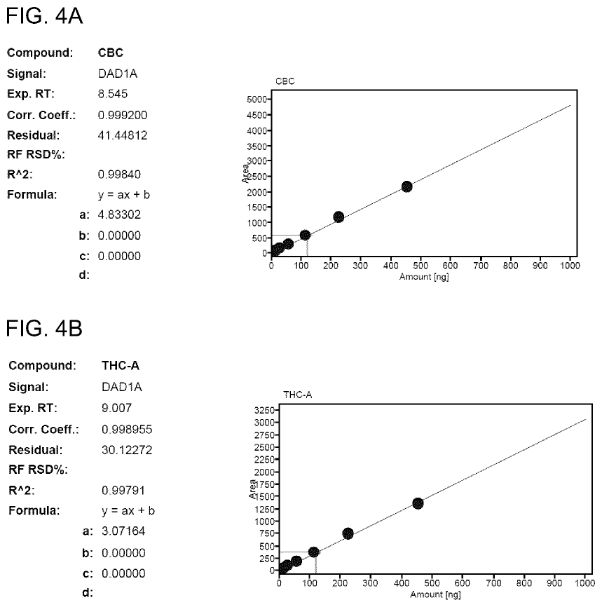

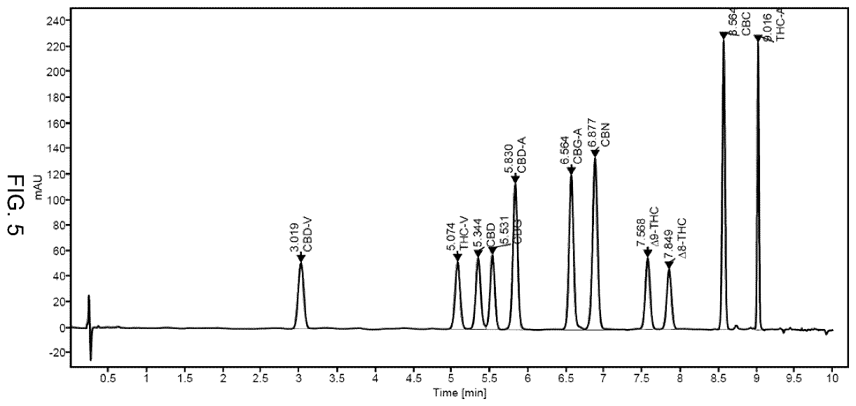

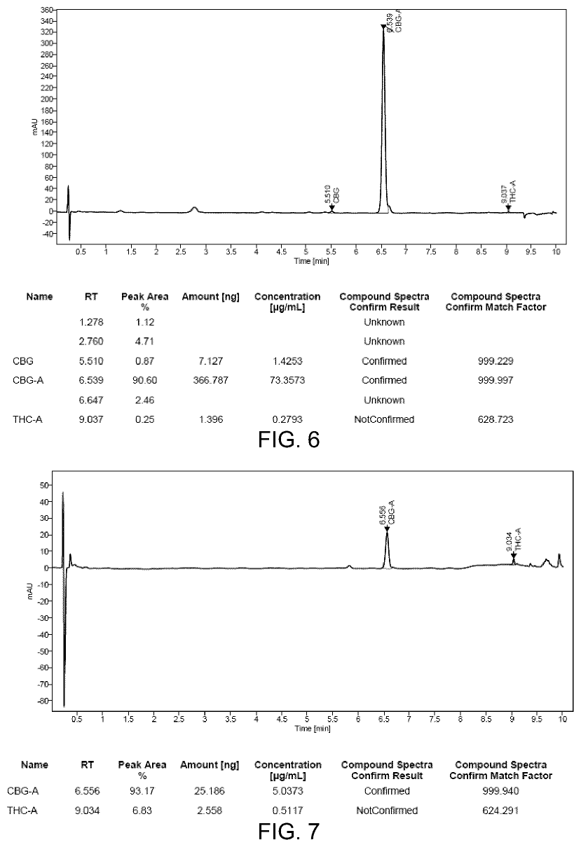

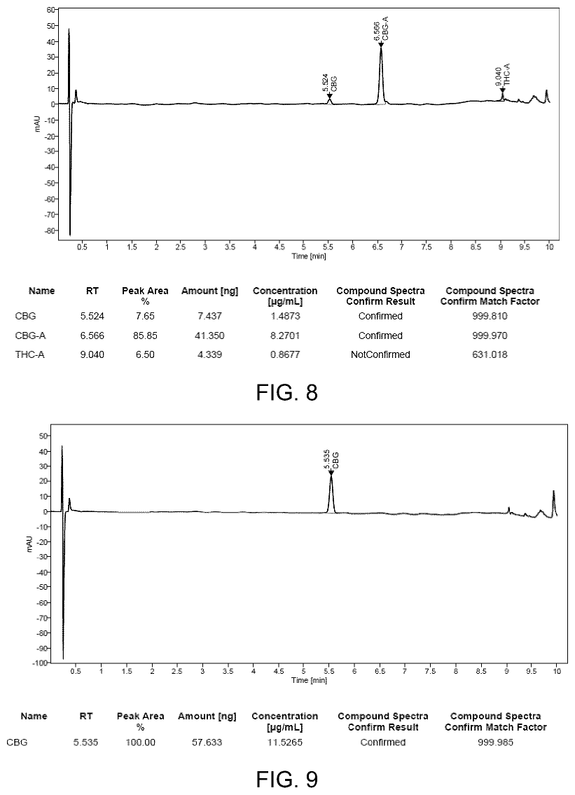

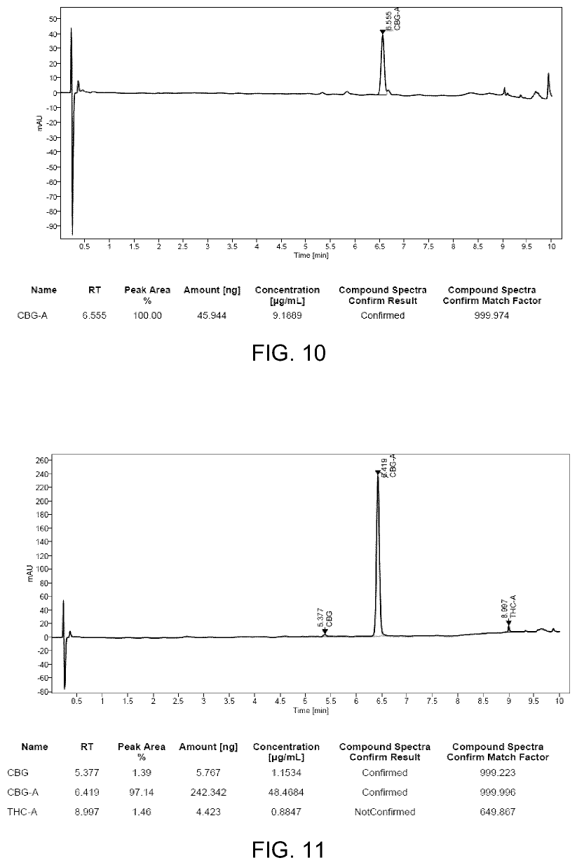

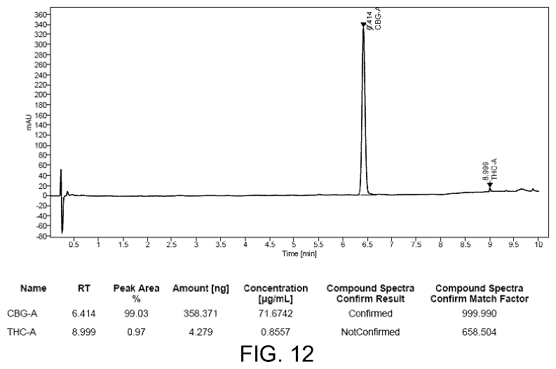

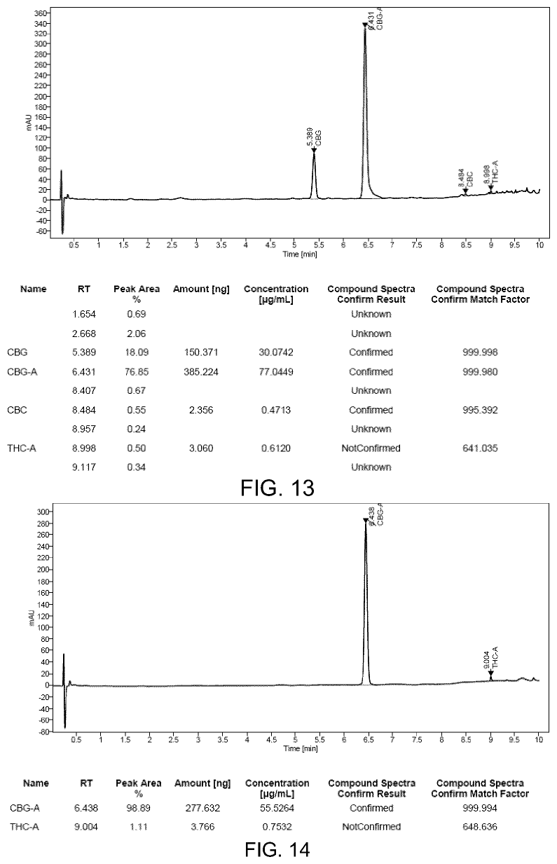

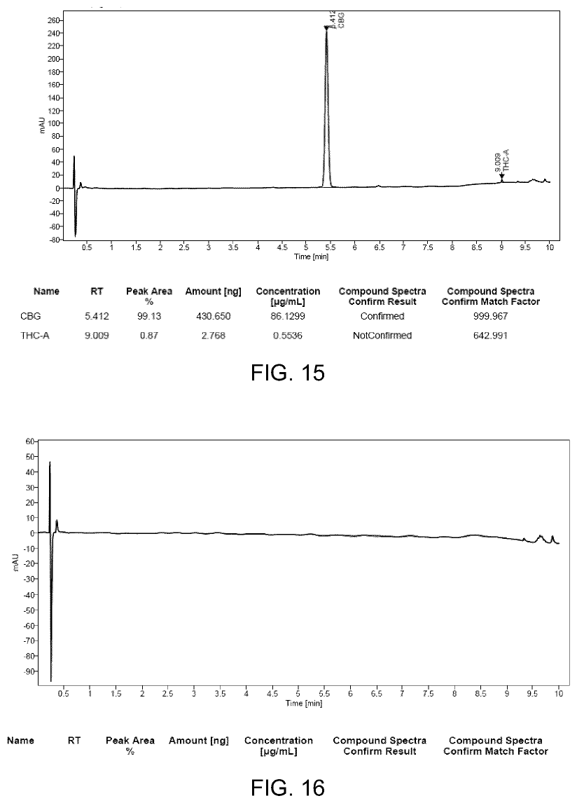

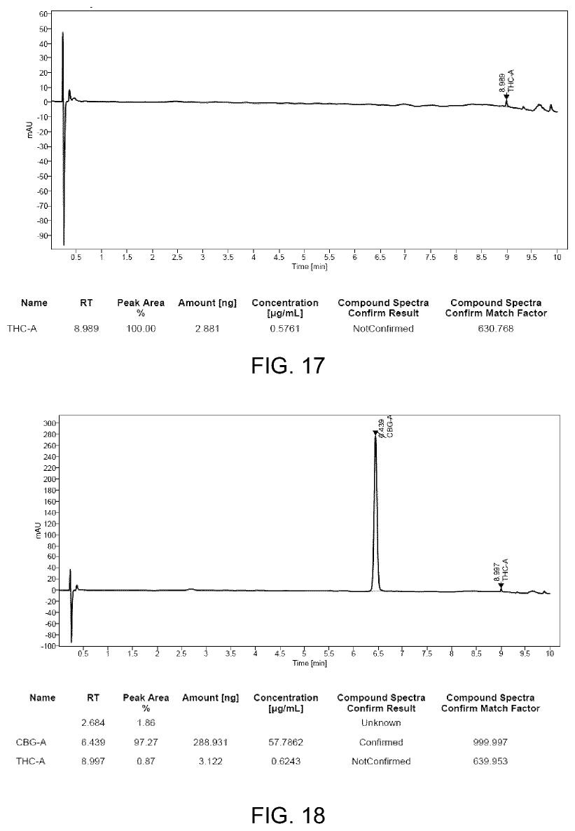

Prior to assessing and refining the methods disclosed herein, an internal method for detecting and quantifying individual THC and CBD phytochemicals based on use of HPLC methods and equipment, was developed and tested for sensitivity, precision, and reproducibility. Eleven naturally occurring purified cannabinoid phytochemical compounds were purchased from Mandel Scientific Inc. (Guelph, ON, CA). Specifically, cannabidivarin (CBDV), tetrahydrocannbidivarin (THCV), cannabidiol (CBD), cannabigerol (CBG), cannabidiolic acid (CBDA), cannabigerolic acid (CBGA), cannabinol (CBN), (-)-trans-Δ⁹-tetrahydrocannabinol (Δ⁹-THC), cannabichromene (CBC), Δ⁸-tetrahydrocannabinolic acid (Δ⁸-THCA). Seven dilutions (1.42 μg/ml, 2.84 μg/ml, 5.68 μg/ml, 11.36 μg/ml, 22.73 μg/ml, 45.45 μg/ml, 90.90 μg/ml) of each cannabinoid standard were prepared and analyzed in triplicate following the instructions in the Agilent Application Note “Dedicated Cannabinoid Potency Testing Using the Agilent 1220 Infinity II LC System” (downloaded from www.agilent.com/chem). The average of the three measurements for each of the seven dilutions was used to create a linear calibration curve for each of the eleven cannabinoid phytochemical compounds: FIG. 1A, CBDV; FIG. 1B, THCV; FIG. 1C, CBD; FIG. 2A, CBG; FIG. 2B, CBD-A; FIG. 2C, CBGA; FIG. 3A, CBN, FIG. 3B, Δ⁹-THC; FIG. 3C, Δ⁸-THC; FIG. 4A, CBC; FIG. 4B, THCA. A mixture containing 22.73 μg/mL of each of the eleven above-noted cannabinoid phytochemical compounds was prepared and then analyzed with the Agilent 1220 Infinity II LC System. The HPLC analysis of the mixture is shown in FIG. 5 and summarized below in Table 1.

TABLE 1

| | | Peak Area | Amount | Concentration |
|---|---|---|---|---|
| Name | RT | % | [ng] | [μg/mL] |
| CBD-V | 3.019 | 6.49 | 114.983 | 22.9965 |
| THC-V | 5.074 | 6.13 | 121.932 | 24.3865 |
| CBD | 5.344 | 6.34 | 121.629 | 24.3257 |
| CBG | 5.531 | 6.24 | 120.126 | 24.0252 |
| CBD-A | 5.830 | 12.32 | 125.316 | 25.0633 |
| CBG-A | 6.564 | 12.75 | 123.143 | 24.6285 |
| CBN | 6.877 | 15.31 | 120.991 | 24.1982 |
| Δ9-THC | 7.568 | 6.12 | 121.963 | 24.3925 |
| Δ8-THC | 7.849 | 5.05 | 118.237 | 23.6473 |
| CBC | 8.564 | 14.13 | 119.110 | 23.8221 |
| THC-A | 9.016 | 9.10 | 120.688 | 24.1376 |

### Example 2

53.8 g of a high CBGA-containing hemp flower was extracted with 9:1 volume ratio of denatured ethanol (84.15% ethanol, 15% methanol, 0.85% ethyl acetate) by first grinding the biomass and then, comingling the ground biomass and ethanol for 25 min at ambient room temperature.

The liquid phase was separated from the biomass by pressure filtration using nitrogen after which, the ethanol was separated from the organic phase by distillation under vacuum to produce 5.6 g of a crude hemp extract resin containing CBGA.

A standardized stock solution of the hemp extract was prepared by dissolving the hemp extract resin in 28 mL denatured ethanol to produce a solvent-solubilized crude hemp extract solution containing 73.357 mg/mL of CBGA (FIG. 6).

A 3:1 molar ratio of N,N-diisopropylethylamine (0.535 mL; Hunig's base) was added to a 5 mL aliquot of the standardized solution and mixed by vortexing. 5 mL of heptane was then added dropwise causing precipitation of a crude CGBA-Hunig's base salt. The mixture was incubated at −20° C. for 10 min, centrifuged for 5 min at 4200 rpm after which, the liquid phase was decanted. The remaining precipitate pellet was resuspended in 5 mL cold heptane and the solid CBGA-Hunig's base salt was separated from the liquid phase by vacuum filtration and dried, yielding 0.6235 g of an off-white solid. A sample of the washed solid CBGA-amine salt was solubilized in HPLC-grade methanol, diluted 250×, and analyzed by HPLC (FIG. 7).

A 50 μL sample of the depleted extract (liquid phase, total vol. 14 mL) was dried under vacuum, diluted 20× in HPLC-grade methanol, and analyzed by HPLC (FIG. 8). The precipitation reaction yield was determined to be 87.4% (total CBGA removed from the standardized extract solution).

### Example 3

### Decarboxylation of CBGA-Hunig's Base Salt

Salt (158 mg) which had been purified by washing the crude precipitate with hexane was dissolved in 9.5 mL of toluene and 0.5 ml of 95% ethanol. The mixture was refluxed for 17 h. TLC indicated the formation of CBG, based on comparison with CBD and some remaining salt. The reaction mixture was washed with 5 mL of 5% aqueous HCl solution and the toluene layer was evaporated. The residue was dissolved in 1 mL of dichloromethane and added to a 3 g of silica gel prepared with 5:1 hexane:ethyl acetate. Elution with 60 ml of this solvent afforded 62 mg of CBG as a solid after removal of the solvent. The product was recrystalized by dissolving it in 1 mL of hexane, cooling to −20° C. and filtering. The ¹H NMR of the resultant white solid, mp. 50-51 C was in agreement with published NMR data for CBG. A sample of the purified solid CBG was solubilized in HPLC-grade methanol, diluted 250×, and analyzed by HPLC (FIG. 9).

### Conversion of CBG-Hunig's Base Salt to CBGA

Recrystallized CBG-Hunig's base salt (55 mg) was dissolved in 3 mL of dichloromethane. The solution was added to a separatory funnel and shaken with 3 mL of 5% HCl solution. The organic phase was dried and the solvent was evaporated to yield 38 mg of CBGA as a white solid. A sample of the washed solid CBGA-amine salt was solubilized in HPLC-grade methanol, diluted 250×, and analyzed by HPLC (FIG. 10).

### Example 4

A screening study was performed to assess the efficiency of five organic solvents for extraction of CBGA from plant biomass:
(i) hexane,
(ii) ethyl acetate,
(iii) dichloromethane,
(iv) 95% ethanol,
(v) acetone.

### 4.1 Hexane

Dried, ground kief produced from a high-CBGA-containing hemp cultivar named “HURV19PAN (Panakiea)” was obtained from Cannabis Orchards Inc. (Ottawa, ON, CA). The Certificate of Analysis provided with the kief indicated its CBG content was 218.65 mg/g (21.87% wt/wt).

17.5 g of the kief was extracted by stirring with 90 mL of hexane at ambient room temperature (˜18° C. to 21° C.) for about 1 h. The mixture was vacuum-filtered after which, the filter cake was washed with an additional 30 mL of hexane. The total volume of filtrate, a golden yellow color, was 110 mL.

10-mL aliquots of the filtrate were separately reacted with about 100 mg of (i) N,N-diisopropylethylamine (Hunig's base), (ii) tributylamine, (iii) triethylamine, and (iv) dimethylaminoethanol. In each case, a minimal amount of cloudiness appeared. The reactions with Hunig's base and tributylamine yielded small amounts of crystalline material. The remaining hexane solution was evaporated to yield 550 mg of a semi-solid. This was taken up in 10 mL of hot hexane and reacted with 1 mL of Hunig's base. The resultant salt was filtered to yield 480 mg of an almost white solid. Based on these observations, it was concluded that hexane does not extract CBG acid efficiently from hemp biomass at ambient temperatures.

The remaining filter cake was re-extracted with 90 ml of 95% ethanol for about 1 h at ambient room temperature. The ethanol-soluble materials was reacted with Hung's base to yield 1.7 g of CBGA-amine salt as an almost white solid crystalline solid.

### 4.2 Ethyl Acetate

5.0 g of the HURV19PAN kief biomass was extracted by stirring with 35 mL of ethyl acetate at room temperature for 1 h. The mixture was filtered and the filter cake was washed with an additional 20 mL of ethyl acetate. The filtrate was evaporated to yield 1.1 g of brown solid. The brown solid was resolubilized in ethyl acetate and then recrystalized by the addition of about 10 mL of hexane The recrystallized material was filtered off yielding 0.35 g of an almost white crystalline material. ¹H NMR analysis of this material confirmed it was CBGA.

### 4.3 Dichloromethane (DCM)

5.0 g of the HURV19PAN kief biomass was extracted with 35 mL of dichloromethane with stirring for 1 h at room temperature. The mixture was filtered and the filter cake was washed with an additional 20 mL of DCM.

The light brown solution was evaporated to yield 1.1 g of a tan solid that was recrystallized by dissolving in a minimum amount of hot hexane and then cooling to −20° C. to yield about 0.92 g of a white crystalline material. ¹H NMR analysis confirmed this white material to be CBGA.

A second 5.0 g sample of the HURV19PAN kief biomass was extracted with 35 mL of DCM while stirring at ambient room temperature for 1 h. The mixture was filtered under suction and the filter cake washed with 25 mL of DCM. 1 mL of Hunig's base was added to the filtrate after which, the solvent was removed by evaporation under vacuum using a Rotovap evaporator. The remaining solid was redissolved in a minimum amount of DCM and 30 mL of hexane was added to thereby cause precipitation of a tan-colored solid. The yield was 1.15 g of CBGA-Hunig's base salt.

35.33 g of the HURV19PAN kief biomass was extracted with 245 mL of DCM while stirring at ambient room temperature for 1 h. The mixture was filtered under suction and the filter cake was washed with 150 mL of DCM. 7 mL of Hunig's base were added to the clear brown solution after which, the solvent and excess base were removed via a rotary evaporator to yield 10.92 g of a dark brown gum. A mixture of 15 mL of ethyl acetate and 75 mL of hexane were added to the gum resulting in formation of a solid. The mixture was placed into a −20° C. freezer for 30 min and then filtered. The solid was washed with 20 mL of hexane to produce a light tan solid crystalline salt material. The salt yield was 8.08 g which is equivalent to 5.95 g CBGA and to 5.22 g of CBG. Accordingly, the concentration of a CBG acid in the HURV19PAN kief biomass sample was at least 16.8% wt/wt.

### 4.4 95% Ethanol

A 5.0 g sample of the HURV19PAN kief biomass was extracted with 35 mL of 95% ethanol while stirring at ambient room temperature for 1 h. The mixture was filtered under suction and the filter cake washed with 25 mL of 95% ethanol. Hunig's base 1 mL was added to the filtrate and the solvent was removed by evaporation on a Rotovap. The remaining solid was redissolved in a minimal amount of DCM and then, 30 mL of hexane were added to thereby cause precipitation of a tan-colored solid crystalline material. The yield was 1.16 g of CBGA Hunig's base salt.

### 4.5 Acetone

A 5.10 g sample of the HURV19PAN kief biomass was stirred with 35 mL of acetone while stirring at ambient room temperature for 1 h. The mixture was filtered under suction and the filter cake was washed with an additional 20 mL of acetone. 1 mL of Hunig's base was added to the filtrate after which, the solvent was removed by evaporation to yield a brownish gum. Addition of 20 mL of a 9:1 mixture of hexane and ethyl acetate resulted in a crystallization occurring on the sides of the flask. The solids were filtered and washed with 10 mL of hexane to yield 1.09 g of tan-colored solid crystalline salt material. The calculations determined that yield was equivalent to 0.80 g (15.7% wt/wt) of CBGA or 0.73 g (14.4% wt/wt) of CBG in the supplied biomass.

The results indicate that ethyl acetate, dichloromethane, and 95% ethanol are particularly suitable organic solvents for extraction of CBGA from Cannabis biomass.

### 4.6 Conversion of CBGA-N,N-diisopropylethylamine Salt (Hunig's Base Amine Salt) to CBG Acid

2.55 g of the CBGA-Hunig's base amine salt produced during the assessment of ethyl acetate as an extracting solvent for use with the HURV19PAN kief biomass, were added to 25 mL of dichloromethane and dissolved. Then, the resulting solution was shaken with 25 mL of a 5% HCl solution. The DCM layer was separated from the aqueous layer, dried with anhydrous magnesium sulfate, after which the DCM was removed by evaporation in a rotovap. The yield was 1.80 g (97%) of a tan-colored solid crystalline material. The melting point (mp) of the crystalline material was 115° C. to 116.° C. ¹H NMR analysis confirmed that the crystalline material was CBGA.

### 4.7 Decarboxylation of CBGA-N,N-diisopropylethylamine Salt (Hunig's Base Amine Salt) in Refluxing 5% Na₂O₃

2.32 g of the CBGA-Hunig's base amine salt produced during the assessment of ethyl acetate as an extracting solvent for use with Cannabis biomass, were suspended in 20 mL of 5% Na₂CO₃ solution. The mixture was refluxed for 4 h after which time, a TLC analysis of the refluxing mixture showed the absence of the acid form. The reaction mixture was allowed to cool and then diluted with 20 mL of hexane. The aqueous and organic layers were separated and the aqueous phase was extracted a second time with 10 mL of hexane. The combined organic hexane layers were washed with 10 mL of a 5% HCl solution, dried over anhydrous MgSO₄, after which, the solvent was removed by evaporation on a rotovap to yield 1.83 g of a crude brownish oil which solidified on standing at room temperature. The CBG yield in the crude oil was 89%.

The crude CBG oil product was purified by passing through 10 g of silica gel followed by elution with 7:1 hexane-ethyl acetate. The yield of CBG as a chromatographically pure white solid crystalline material was 1.52 g (75%). The mp of the crystalline material was 52° C. to 53° C. 1H NMR analysis confirmed that the crystalline material was CBG.

### Example 5

A study was performed to assess the potential of twenty-four selected amine compounds from a range of amines, for reliable and routine precipitation of CBGA from complex mixtures.

Each of the twenty-four amines listed in Table 2 was assessed for its potential to crystallize (i.e., precipitate) CBGA from a selected organic solvent solution by dropwise addition of the amine into a solubilized CBGA solution to provide a 50% molar excess of the amine.

Each of the amines was dissolved in 2.5 mL hexane. Amines that were not soluble in hexane, were solubilized in 2.5 mL of ethyl acetate. For the reactions with the amines in ethyl acetate, an additional 5 mL of hexane was added into the reaction mixtures. It is to be noted that the molecular weight of CBG is 316.5, and that the molecular weights for the amines tested in this study were in a range of 100 to 150. Accordingly, the yields expected were in the range of 80% to 90% of the theoretical yield (theoretical yields in a range of 450 to 500 mg), that is, about 400 mg.

Nine of the twenty-four amines assessed in this study precipitated CBGA as an amine salt from an organic solvent solution containing 100 mg of CGBA (Table 2). The CBGA-salt precipitating amines were:
(i) N,N-diisopropylethylamine (Hunig's base),
(ii) dicyclohexylamine,
(iii) methyldicyclohexylamine,
(iv) 1,4-diazabicyclo[2.2.2]octane (DABCO),
(v) triethylamine,
(vi) tripropylamine,
(vii) tributylamine,
(viii) Isopropycyclohexylamine, and
(ix) 2,2,6,6-tetramethylpiperidine.

TABLE 2

| | | Crystals | MP |
|---|---|---|---|
| | Amine | formed | (° C.) |
| | Aromatic amines | | |
| 1 | tert-butylamine | no | |
| 2 | methylbenzylamine | no | |
| | Secondary amines | | |
| 3 | pyrrolidine | no | |
| 4 | diisopropylamine | no | |
| 5 | dicyclohexylamine | no | |
| 6 | isopropylcyclohexylamine | no | |
| 7 | dicyclohexylamine | YES | 160-163 |
| 8 | isopropylcyclohexylamine | YES | 91-92 |
| 9 | 2,2,6,6-tetramethylpiperidine | YES | 136-139 |
| 10 | morpholine | no | |
| 11 | piperidine | no | |
| | Tertiary amines | | |
| 12 | triethylamine | YES | 75-77 |
| 13 | tripropylamine | YES | 110-112 |
| 14 | tributylamine | YES | 118-122 |
| 15 | N,N-diisopropylethylamine (Hunig's base) | YES | 119-120 |
| 16 | N-Methyldicyclohexylamine | YES | 112-116 |
| | Amino alcohols | | |
| 17 | dimethylethanolamine (DMEA) | no | |
| 18 | piperidineethanol | no | |
| | Diamines | | |
| 19 | 1,8-diazabicyclo[5.4.0]undec-7-ene (DBU) | no | |
| 20 | 1,4-diazabicyclo[2.2.2]octane (DABCO) | YES | 103-105 |
| 21 | tetramethylethylenediamine (TMEDA) | no | |
| 22 | N-methylpiperazine | no | |
| 23 | dimethylaminopyridine (DMAP) | no | |
| | Aromatic amines | | |
| 24 | aniline | no | |

In each of the reaction vessels wherein a selected amine caused CBGA crystallization/precipitation, the remaining solution was analyzed with thin-layer chromatography to determine if any CBGA remained in solution. In all cases wherein CBGA crystallization/precipitation occurred, there weren't any significant amounts of CBGA remaining in solution indicating that essentially all of the CBGA had been crystallized/precipitated.

Each of the amine salt products was filtered to remove excess amine solution, and then washed with a small volume of hexane. Each of the dried salt products was weighed and its melting point (MP) determined. Most of the measured melting points (MP) were quite narrow indicating high purity of the precipitated CBGA salt (Table 2).

The nine room-temperature solid CBGA-amine salts produced were characterized by taking their ¹H NMR spectra in CDCl₃ and recording at 400 MHz. Each of the CBGA-amine salts showed the expected peaks due to the ammonium ion in addition to all the peaks comprising the CBGA acid unit. The integration of the peaks was consistent with a 1:1 ratio of ammonium ion vs CBGA carboxylate. Key peaks of the carboxylate portion, see structure (1) below, and key peaks due to the ammonium ion which do not overlap with the CBGA carboxylate peaks are reported. The seven carboxylate peaks are listed first starting with the most deshielded peak due to H1 and ending with peaks to the methyl group 7 are reported in bold in the spectroscopic data. These peaks are found at 6.24 (s, 1H), 5.27 (m, 1H), 5.04 (m, 1H), 1.80 (s, 3H), 1.66 (s, 3H), 1.57 (s, 1H) and 0.83 (t, 3H) ppm in CBG acid. The peak assignment and the integration of the relevant ammonium ion peaks are also given.

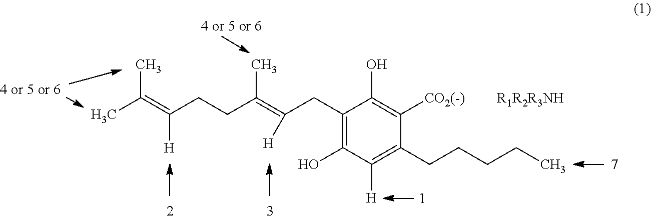

¹H NMR (400 MHz, CDCl₃ solvent, δ (ppm)): Key peaks due to CBGA: 6.12 (s, 1H), 5.30 (m, 1H), 5.04 (m, 1H), 3.42 (t, J=7.6 Hz, 2H), 1.78 (s, 3H) 1.64 (s, 3H), 1.60 (s, 3H), 0.83 (t, J=3H).

Key peaks due the ammonium ion. δ (ppm): 3.67 (quint, 2H), 3.04 (qt, 2H), 1.37 (d, J=6.8 H, 6H).

¹³C NMR δ (ppm): 174.90. 162.39, 156.8, 145.82, 137.29, 131.71, 124.08, 122.90. 110.43, 110.35, 108.43, 52.76, 41.38. 39.78, 35.73, 32.27, 31.77, 25.54, 22.85, 22.43., 18.11, 17.69. 16.20, 14.16, 11.80.

The ¹³C spectra of the CBGA-amine salts all show the expected number of unique carbon signals.

### Salt 1: CBGA-N,N-diisopropylethylamine Salt (Hunig's Base)

5.0 g of CBGA-containing hemp biomass extracted with 35 mL of DCM while stirring for 60 min, followed by separation of the solvent from the biomass by filtration. The filter cake was then washed with 25 mL of DCM and the two filtrates were combined. 1.0 mL of Hunig's base was added to the filtrate and stirred for 30 min. The solvent was then evaporated using a Rotovap rotary evaporator to thereby produce a crude CBGA-Hunig's base salt. The salt was resolubilized in a small volume of DCM and recrystallized to yield 1.15 g of purified CBGA-Hunig's base salt with a melting point of 119-120° C..

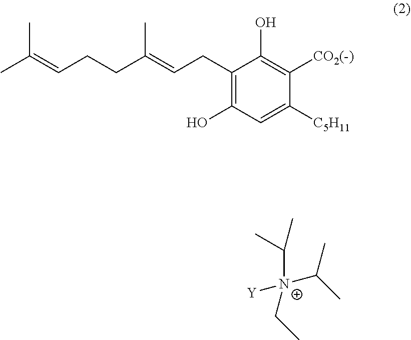

¹H NMR (400 MHz, CDCl₃ solvent, δ (ppm): Key peaks due to CBGA: 6.12 (s, 1H), 5.30 (m, 1H), 5.04 (m, 1H), 2H), 3.42 (t, J=7.6 Hz, 2H), 1.78 (s, 3H), 1.64 (s, 3H), 1.60 (s, 3H), 0.83 (t, J=3H)

Key peaks due to the ammonium ion. δ (ppm): 3.67 (quint, 2H), 3.04 (qt, 2H), 1.37 (d, J=6.8 H, 6H).

¹³C NMR δ (ppm): 174.90. 162.39, 156.8, 145.82, 137.29, 131.71, 124.08, 122.90. 110.43, 110.35, 108.43, 52.76, 41.38. 39.78, 35.73, 32.27, 31.77, 25.54, 22.85, 22.43., 18.11, 17.69. 16.20, 14.16, 11.80.

### Salt 2: CBGA-dicyclohexylamine Salt

A hexane solution containing 100 mg of CBG acid, isolated by ethyl acetate extraction was reacted with 100 mg of dicyclohexylamine, and yielded 93 mg CBGA-dicyclohexylamine salt. The melting point of the salt was 160-163° C.

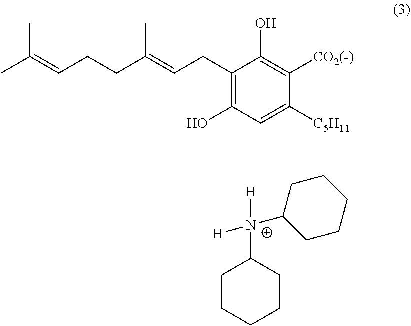

¹H NMR (400 MHz, CDCl₃ solvent, δ (ppm)): Key peaks due to CBGA: 6.12 (s, 1H), 5.31 (m, 1H), 5.04 (m, 1H), 3.41 (t, J=7.6 Hz, 2H), 1.78 (s, 3H), 1.64 (s, 3H), 1.56 (s, 3H), 0.83 (t, J=3H).

Key peaks due the ammonium ion. δ (ppm): 3.13 (m, 2H), 2.63 (s, 3H).

### Salt 3. CBGA-methyldicyclohexyamine Salt

100 mg of methyldicyclohexylamine was dissolved in 1 mL of hexane and then added to 15 mL of hexane containing 110 mg of CBGA. A white oil separated which was converted to 132 mg of a white solid upon scratching the side of the container. The melting point of the salt was 112-116° C.

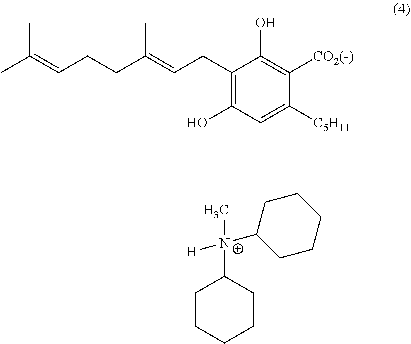

¹H NMR: (400 MHz, CDCl₃). δ (ppm) values for key peaks due to CBGA: 6.13 (s, 1H), 5.30 (t, 1H), 5.04 (t, 1H), 3.41 (d, J=7.2 H, 2H), 3.0 (t, J=7.8 Hz, 2H), 1.78 (9S, 3H), 1.64 (s, 3H), 1.59 (s, 3h), 0.84(t, 3H).

Key peaks due to the ammonium ion: 3.14(2H) 2.63 (s, 3H).

### Salt 4. CBGA-1,4-diazabicyclo[2.2.2]octane (DABCO)

A 3:1 hexane-DCM solution containing 210 mg of CBG acid was treated with 150 mg of DABCO. An oil began to form at the bottom of the conical flask when the DCM began to evaporate selectively. Scratching the oil against the side of the flask induced crystallization. The crystals were filtered off and washed with diethyl ether to help remove any remaining DABCO to yield 273 mg CBGA-DABCO salt. The melting point of the salt was 103-105° C.

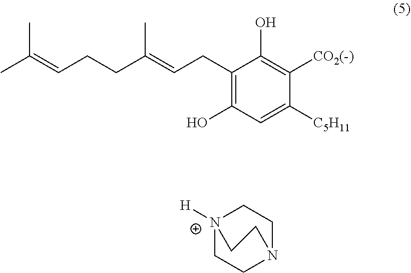

¹H NMR: (400 MHz, CDCl₃). δ (ppm) values for key peaks due to CBGA: 6.13 (s, 1H), 5.30 (t, 1H), 5.04 (t, 1H), 3.41 (d, J=7.2 H, 2H), 3.0 (t, J=7.8 Hz, 2H), 1.78 9S, 3H), 1.64 (s, 3H), 1.59 (s, 3h), 0.84 (t, 3H).

Single peak due to the ammonium ion: δ (ppm): 2.09

### Salt 5. CBGA-triethylamine Salt

47 mg of pure CBGA was dissolved in 1 ml of DCM after which, 4 drops of triethylamine were added followed by 4 mL of hexane. Evaporation of the solvents produced 35 mg of CBGA-triethylamine salt. The melting point of the salt was 75-77° C.

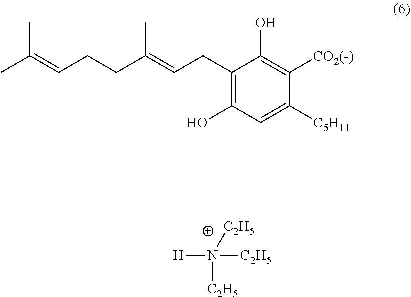

¹H NMR: (400 MHz, CDCl₃). δ (ppm) values for key peaks due to CBGA: 6.13 (s, 1H), 5.30 (m, 1H), 5.04 (m, 1H), 3.41 (d, J=6.4 Hz, 2H), 1.78 (s, 3H) 1.65 (s, 3H), 1.56 (s, 3H), 0.84 (t, J=3H).

Key peaks due to the ammonium ion. 8 (ppm): 3.06 (q, 6H), 1.29(t, 9H).

### Salt 6. CBGA-tripropylamine Salt

1.15 g of CBGA was dissolved in 23 mL of ethyl acetate (50 mg/mL). The solution was treated with tripropylamine was added to the solution which precipitated a salt. The solvent was evaporated after which, hexane was added to resolubilize the salt. Evaporation of the hexane yielded 102 mg of a CBGA-tripropylamine salt. The melting point of the salt was 110-112° C.

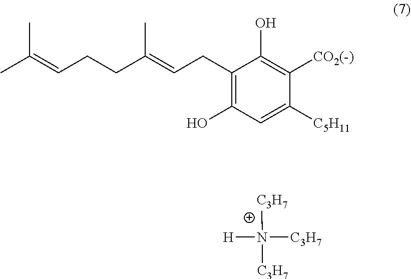

¹HNMR: (400 MHz, CDCl₃). δ (ppm) values for key peaks due to CBGA: 6.13 s, 1H), 5.30 (m, 1H), 5.04 (m, 1H), 3.4 (d, 2H), 1.78 (s, 3H) 1.64 (s, 3H), 1.58 (s, 3H), 0.83 (t, 3H)

Key peaks due to the ammonium ion. δ (ppm): 2.92 (m, 6H), 0.94 (t, 9H).

### Salt 7. CBGA-tributylamine Salt

1.15 g of CBGA was dissolved in 23 mL of ethyl acetate (50 mg/mL). Tributylamine was added to the solution which precipitated a salt. The solvent was evaporated after which, the resulting amine salt was washed with hexane. Evaporation of the hexane yielded 1119 mg of a CBGA-tributylamine salt. The melting point of the salt was 118-122° C.

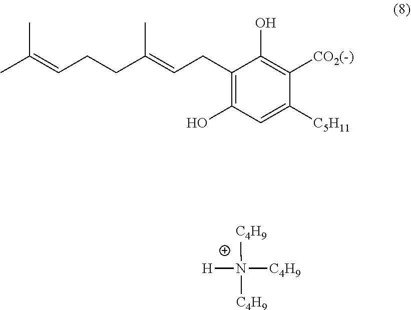

¹H NMR: (400 MHz, CDCl₃). δ (ppm) values for key peaks due to CBGA: 6.13 (s, 1H), 5.31 (m, 1H), 5.04 (m, 1H), 2H), 3.42 (t, J=7.6 Hz, 2H), 1.78 (s, 3H), 1.65 (s, 3H), 1.60 (s, 3H), 0.85 (t, J=3H).

Key peaks due to the ammonium ion. δ (ppm): 2.9 (m, 6H), 0.93 (t, J=7.6 Hz, 9H).

### Salt 8. CBGA-isopropycyclohexylamine Salt

50 mg of pure CBGA were dissolved in 2 ml of dichloromethane. Then, 4 drops of isopropylcyclohexylamine were added followed by 4 mL of hexane. Evaporation of the solvents produced 45 mg of CBGA-isopropycyclohexylamine salt. The melting point of the salt was 91-92° C.

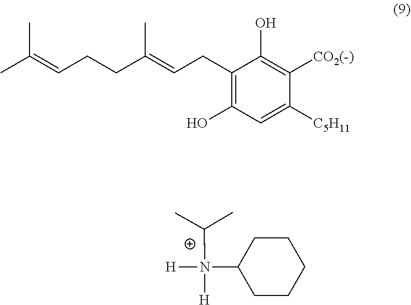

¹H NMR: (400 MHz, CDCl₃). δ (ppm) values for key peaks due to CBGA: 6.16 (s, 1H), 5.30 (m, 1H), 5.04 (m, 1H, 2H), 3.41 (m, 2H), 1.79 (s, 3H), 1.65 (s, 3H), 1.57 (s, 3H), 0.85 (t, J=3H).

Key peaks due to the ammonium ion. δ (ppm): 3.18 (m, 2H), 1.19 (d, J=6.0 Hz, 6H).

The ¹³C NMR spectra showed the required 17 unique sp3 carbon resonances. CDCl₃). δ (ppm): 53.6, 45.3. 39.8, 35.9, 32.4, 319, 31.3, 26.5, 25.7, 25.6, 25.1, 22.9, 22.4, 21.0, 17.7, 16.2, 14.2.

### Salt 9. CBGA-2,2,6,6-tetramethylpiperidine

37 mg of pure CBGA were dissolved in 2 ml of dichloromethane. Then, 4 drops of 2,2,6,6 tetramethylpiperidine were added followed by 4 mL of hexane. Evaporation of the solvents resulted in 43 mg of a CBGA-2,2,6,6-tetramethylpiperidine salt. The melting point of the salt was 136-139° C.

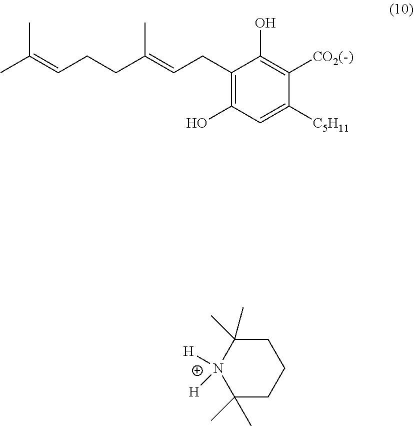

¹H NMR: (400 MHz, CDCl₃). δ (ppm) values for key peaks due to CBGA: 6.18 (s, 1H), 5.31 (t, 1H), 5.04 (t, 1H, 2H), 3.42 (d, J=7.6.8 Hz, 2H), 1.79 (s, 3H) 1.65 (s, 3H), 1.56 (s, 3H), 0.84 (t, J=3H).

Key peak due to the ammonium ion: δ (ppm): 2.9 (1.45 (s, 12H)

### Example 6

This study assessed the extraction and recovery of a CBGA-amine salt from a high-CBGA producing cultivar of hemp, followed by purification of the CBGA-amine salt, and decarboxylation of the CBGA-amine salt into CBG.

A quantity of a dried and powdered kief from a high-CBGA producing hemp cultivar named “HURV19PAN” (Panakiea) was obtained from Cannabis Orchards Inc. (Ottawa, ON, CA). The certificate of analysis for its cannabinoid content is shown in Table 3.

96.6 g of the HURV19PAN hemp kief were extracted with 966 mL of cold ethyl acetate (−20° C.) for 20 min after which the ethyl acetate extract was recovered by pressure filtration. The kief filter cake was washed by pressure filtration with 266 mL of fresh cold ethyl acetate (−20° C.) after which, the two filtrates were combined.

The ethyl acetate solvent was removed from the filtrate by distillation with a rotary evaporator to thereby produce 18.8 g of resin comprising a crude complex mixture of phytochemicals recovered from the HURV19PAN hemp kief. The crude complex resin was resolubilized in 75.2 mL of ethyl acetate to produce a 4:1 standardized crude extract.

TABLE 3

| | LOD* | LOQ** | | | |
|---|---|---|---|---|---|
| Analyte | (μg/g) | (μg/g) | Results | | |
| Tetrahydrocannabinolic | 7.44 | 22.48 | | | |
| acid (THCa) | | | | | |
| Cannabinol (CBN) | 8.52 | 25.84 | | | |
| Cannabidiolic acid (CBDa) | 2504 | 7.6 | | | |
| Cannabigerolic | 3.844 | 11.64 | | | |
| acid (CBGa) | | | | | |
| Δ9-Tetrahydrocannabinol | 6.0 | 18.2 | | | |
| (Δ9-THC) | | | | | |
| Cannabigerol (CBG) | 8.16 | 24.76 | 21.87% | 28.65 | mg/g |
| Cannabidiol (CBD) | 6.96 | 21.04 | | | |
| Total CBD | | | 0.0% | 0.0 | mg/g |
| (CBDa * 0.877 + CBD) | | | | | |
| Total THC | | | 0.0% | 0.0 | mg/g |
| (THCa * 0.877 + THC) | | | | | |

*LOD is the smallest concentration of an analyte that can be detected.
**LOQ is the smallest concentration of an analyte that can be determined with acceptable repeatability and accuracy

A 20-μL aliquot of the standardized crude extract was prepared for HPLC analysis as follows. First, the 20-μL aliquot was transferred into to a 1.5 mL microfuge tube. The solvent was evaporated from the sample by vacuum centrifugation to produce a resin. The resin was resuspended in 1,000 μL of HPLC-grade methanol at 40° C. to create a 50×-diluted sample. The resuspended Cannabis resin was further diluted 50× solution to a 250× final dilution by transfer to a new 1.5 ml microfuge tube to which was added 980 μL of HPLC-grade methanol and mixed well. The 250× final diluted sample was transferred into a HPLC sample vial with a 0.45 μm syringe fitted with a filter. A 5 μl aliquot of the 250× diluted sample was injected into an HPLC and analyzed in reference to the calibrated 7-point Cannabinoid HPLC analytical method disclosed in Example 1 to determine that the 4:1 ethyl acetate-standardized crude complex extract contained 11.154 g of CBGA (FIG. 11). The total molar content of CBGA in the standardized crude extract was 3.09×10⁻² mol. The sample was analyzed with an Agilent 1220 II Infinity LC Gradient UV/DAD High-Pressure Liquid Chromatography System (HPLC) following the instructions in the Agilent Application Note “Dedicated Cannabinoid Potency Testing Using the Agilent 1220 Infinity II LC System” (downloaded from www.agilent.com/chem).

A crude CBGA-amine salt was precipitated from standardized crude extract with N,N-diisopropylethylamine (Hunig's base). First, the resolubilized standardized crude extract has heated to and maintained at 30° C. to which was added a 3:1 molar ratio of Hunig's base under constant mixing. When precipitation was observed from the standardized crude extract the reaction mixture was spiked with a 1.75:1 ratio (vol/vol) of n-heptane while maintaining the constant mixing. The crude CBGA-Hunig's base amine salt was separated from the heptane-spiked standardized crude extract mixture by vacuum filtration. The crude CBGA-Hunig's base amine salt was washed by resuspension and mixing in cold n-heptane using a 5:1 volume/mass ratio, then recovered again by vacuum filtration, and dried to produce 18.98 g of the crude CBGA-Hunig's base amine salt. Samples of the crude CBGA-Hunig's base amine salt and the CBGA-depleted standardized crude extract were reserved for HPLC analysis.

The crude CBGA-amine salt was purified by recrystallization as follows. The dried crude CBGA-amine salt was added into 76 mL of denatured ethanol at a 4:1 vol/wt ratio and warmed to 43° C. under constant mixing until the salt was completely dissolved. The solution was cooled under constant mixing until its temperature was about 36° C. Then, room-temperature n-heptane was added to the cooled CBGA-amine solution under constant mixing to a final ratio of 1:1 vol/vol n-heptane:denatured ethanol after which, the mixture was cooled to about 4° C. under constant mixing while the purified CBGA-Hunig's base amine salt recrystallized. The purified recrystallized CBGA-Hunig's base amine salt was recovered from the reaction mixture by vacuum filtration and then dried under vacuum to produce 16.33 g of purified CBGA-amine salt. 3 mg of the purified CBGA-amine salt was reserved for HPLC analysis.

The purified CBGA-Hunig's base amine salt was then decarboxylated by adding the amine salt into a rotary evaporator flask with a 2.5% solution of sodium carbonate (Na₂CO₃) in a 10:1 vol/wt ratio, followed by heating to about 101° C. and refluxing under a nitrogen atmosphere while constantly mixing the solution for 4 h to thereby produce a biphasic mixture of decarboxylated CGB oil and aqueous Na₂CO₃. After cooling, a 70-mL volume of n-heptane was added into the mixture which was then stirred to solubilize the CGB oil into the n-heptane solvent. The bi-phasic mixture was transferred into a separatory funnel and then the lower aqueous layer was removed. Then, a 5% HCl solution was added to the n-heptane containing the dissolved CBG oil to a 1:1 ratio vol/vol and then shaken to thereby produce a bi-phasic mixture. The lower aqueous phase containing the 5% HCl plus amine solution was removed, and a second volume of the 5% HCl solution was added to the n-heptane containing the dissolved CBG oil to a 1:1 ratio vol/vol and then shaken to thereby produce another bi-phasic mixture. The lower aqueous phase containing the 5% HCl solution and residual amine was removed from the separatory funnel after which, the organic layer containing the solubilized neutral CBG was transferred to a flask and dried over MgSO₄. The dried organic layer was filtered and transferred to a rotary evaporator flask for removal of the n-heptane solvent by distillation to thereby produce 9.56 g of decarboxylated CBG oil.

The samples reserved from:
(i) the standardized crude extract solution
(ii) the crude CBGA-Hunig's base amine salt precipitated from the crude extract solution,
(iii) the CBG-depleted standardized crude extract solution,
(iv) the purified recrystallized CBGA-Hunig's base amine salt, and
(v) the decarboxylated CBG oil,
   were prepared for HPLC analyses by placing 1-2 mg of each sample into separate 1.5-mL microfuge tubes, adding 1,000 μL of HPLC-grade methanol into each tube followed by vigorous mixing. Sample dilution factors were calculated by multiplying the mass of each sample in mg ×25/3. Each sample was diluted by its dilution factor to a final volume of 1,000 in HPLC-grade methanol. Each sample was then transferred to a HPLC sample vial with a syringe fitted with a 0.45-μm filter. 5 μL of each sample was analyzed with an Agilent 1220 II Infinity LC Gradient UV/DAD High-Pressure Liquid Chromatography System (HPLC) following the instructions in the Agilent Application Note “Dedicated Cannabinoid Potency Testing Using the Agilent 1220 Infinity II LC System” (downloaded from www.agilent.com/chem), in reference to the calibrated 7-point cannabinoid HPLC analytical method disclosed in Example 1.

The HPLC analyses of the (i) standardized crude extract solution (ii) the crude CBGA-Hunig's base amine salt precipitated from the standard crude extract solution, (iii) the CBGA-depleted standardized crude extract solution, (iv) the purified recrystallized CBGA-Hunig's base amine salt s, and (v) the decarboxylated CBG oil are shown in FIGS. 11, 12, 13, 14, 15, respectively.

It should be noted that FIGS. 11 to 15 for Example 6 show a very small peak at a retention time of 9 min that is identified as “THC-A”. This peak is not due to the presence of THC-A but instead, is an unknown anomaly associated with the filtration syringes used to prepare the samples for HPLC analyses. FIG. 16 shows an HPLC scan of an unfiltered HPLC-grade methanol control blank. FIG. 17 shows an HPLC scan of an filtered HPLC-grade methanol control blank wherein an anomaly associated with the filter is mis-identified as “THC-A” by the HPLC.

### Example 7

2.069 g of a crude CBGA-N,N-diisopropylethylamine amine salt (Hunig's base) was precipitated from a standardized crude extract solution prepared by solubilizing a resin comprising a complex mixture of phytochemicals including cannabinoids in ethyl acetate as the organic solvent. The resin was previously prepared from an ethyl acetate crude extract recovered from a biomass sample of the dried and powdered kief from the high-CBGA-producing “HURV19PAN” as disclosed in Example 4.

The CBGA-Hunig's base amine salt amine salt was decarboxylated by the addition of a 2.5% Na₂CO₃ solution (41.3 mL) to a 20:1 vol/wt ratio, followed by refluxing of the reaction mixture at 100-101° C.) for 4 h to thereby produce a bi-phasic mixture consisting of an upper organic oil layer containing CBG and lower aqueous layer containing the Na₂CO₃ solution. The bi-phasic solution was cooled to 35.8° C. after which, 20 mL of n-heptane were added and vigorously mixed and then, the lower aqueous layer was removed. After removal of the lower aqueous layer, the upper organic layer was washed twice with 20 mL of a 5% HCl solution, and then with a third wash with 30 mL of a 200N NaCl solution. The organic layer containing the solubilized neutral CBG was transferred to a flask and dried over MgSO₄. The dried organic layer was filtered and transferred to a rotary evaporator flask for removal of the n-heptane solvent by distillation to thereby produce 0.98 g of decarboxylated CBG oil.

Samples of (i) the crude CBGA-N,N-diisopropylethylamine amine salt, and (ii) the decarboxylated CBG oil were prepared for HPLC analyses following the steps disclosed in Example 6 to produce the HPLC analyses shown in FIGS. 18 and 19, respectively.

It should be noted that FIGS. 18 and 19 for Example 7 show a very small peak at a retention time of about 9 min that is identified as “THC-A”. This peak is not due to the presence of THC-A but instead, is an unknown anomaly associated with the filters in the syringes used to prepare the samples for HPLC analyses (FIGS. 16, 17).

### Example 8

This example assessed methods for extracting, recovery, and purification of crude CBGA-amine salts from a dried-down complex crude mixture of phytochemicals recovered from Cannabis biomass wherein ethyl acetate was the organic solvent wherein to the dried-down complex crude mixture was resolubilized and standardized prior to addition of an amine to thereby precipitate a crude CBGA-amine salt.

A sample of HURV19PAN hemp kief was extracted with cold ethyl acetate (−20° C.) after which the crude complex ethyl acetate extract solution was recovered following the method disclosed in Example 6. A dried resin comprising a complex mixture of compounds was produced by evaporation of the ethyl acetate following the method disclosed in Example 6 after which, the resin was resolubilized in fresh ethyl acetate to form a standardized crude extract solution.

Three 5-mL aliquots of the standardized CBGA hemp extract solution containing 131.056 mg/mL CBGA (FIG. 20) were heated to 30° C. under constant stirring with a magnetic stirrer. Then, a 3:1 molar ratio of N,N-diisopropylethylamine (Hunig's base; 1.106 mL) was added dropwise to each of the three aliquots. N-heptane was then added to two of the three aliquots in a 1:1 vol/vol ratio (5 mL) or 1.75:1 vol/vol ratio (8.75 mL) ratio of heptane:ethyl acetate standardized extract solution. The three reaction mixtures were cooled slowly to 23° C. to thereby cause precipitation of a crude CBGA-Hunig's base amine salt in each of the reaction mixtures.

Each of the three crude CBGA-Hunig's base amine salt precipitate was separated from the organic phase by vacuum filtration, washed with cold heptane, and dried under vacuum.

A minimum quantity of dichloromethane (DCM) was added to the CBGA-depleted extract filtrate/heptane wash solutions to dissolve all solids that formed in the filtrate from the addition of heptane during the wash. The CBGA contents of the CBGA-depleted extract/heptane wash/DCM filtrate solutions were quantified by removing a 50-uL sample (30 uL sample, 1:1 heptane ratio aliquot), separating the ethyl acetate/heptane/DCM under vacuum, dissolving the resulting resin in 1 mL of HPLC-grade methanol and further preparing an undiluted (1:1 heptane aliquot) or 2× dilution (1.75:1 heptane aliquot & neat EA aliquot) of the sample in HPLC-grade methanol. The diluted samples were analyzed by HPLC and the crude CBGA-HB salt precipitation yields in the three samples were determined to be (i) aliquot 1, 92.59% (1:1 heptane; FIG. 21), (ii) aliquot 2, 96.32% (FIG. 22, 1:1.75 heptane), and (iii) aliquot 3, 89.39% (neat EA; FIG. 23).

It should be noted that FIGS. 20 to 23 for Example 8 show a very small peak at a retention time of about 9 min that is identified as “THC-A”. This peak is not due to the presence of THC-A but instead, is an unknown anomaly associated with the filters in the syringes used to prepare the samples for HPLC analyses (FIGS. 16, 17).

### Example 9

This example assessed methods for extracting, recovery, and purification of crude CBGA-amine salts from a dried-down complex crude mixture of phytochemicals recovered from Cannabis biomass wherein 2-propanol was the organic solvent wherein to the dried-down complex crude mixture was resolubilized and standardized prior to addition of an amine to thereby precipitate a crude CBGA-amine salt.

A sample of HURV19PAN hemp kief was extracted with cold ethyl acetate (−20° C.) after which the crude complex ethyl acetate extract solution was recovered following the method disclosed in Example 6. A dried resin comprising a complex mixture of compounds was produced by evaporation of the ethyl acetate following the method disclosed in Example 6 after which, the resin was resolubilized in 2-propanol to form a standardized crude extract solution.

The CBGA content in the standardized crude extract 2-propanol solution was quantified by removing a 10-uL sample of the standardized crude extract solution, separating the 2-propanol under vacuum, dissolving the resulting resin in 1 mL of HPLC-grade methanol and further preparing a 20× dilution of the sample in HPLC-grade methanol. The 20× diluted sample was then analyzed by HPLC and the standardized crude extract solution in 2-propanol was determined to contain 121.146/mL CBGA (FIG. 24).

Two 5-mL aliquots of the standardized crude extract solution in 2-propanol were heated to 30° C. after which, a 3:1 molar ratio of N,N-diisopropylethylamine (Hunig's base; 0.878 mL)] was added dropwise to each aliquot while mixing with a magnetic stir bar. Then, heptane was added in (i) a 1:1 ratio vol./vol. or (ii) a 1.75:1 ratio vol./vol. of heptane to 2-propanol standardized extract to thereby cause precipitation of a crude CBGA-Hunig's base amine salt. The solid CBGA-Hunig's base amine salt was separated from the liquid phase by vacuum filtration, washed with cold heptane, vacuum filtered, and dried. HPLC analyses of the precipitated salts confirmed that the yield in the aliquot receiving a heptane spike at a 1:1 ratio vol./vol. was 91.57% (FIG. 25), whereas the yield in the aliquot receiving a heptane spike at a 1.75:1 ratio vol./vol. was 91.54% (FIG. 26).

Two 0.82 g samples of a solid crude CBGA-Hunig's base amine salt were recrystallized by fully dissolving in 10:1 ratio vol/wt. ratio of 2-propanol (8.2 mL) at 60° C. with constant stirring. Crystallization was induced by slowly cooling the solutions to 38° C. One of the two samples was then further cooled to 4° C. to complete the crystallization process (neat isopropanol). The other of the two samples was spiked with heptane to a 1:1 ratio vol./vol. (heptane:isopropanol) at ambient temperature and then further cooled to 4° C. The recrystallized CBGA-Hunig's base amine salts were separated from the liquid phase by vacuum filtration, washed with 4 mL room-temperature heptane, and dried under vacuum, The sample that was spiked with heptane yielded 0.722 g of purified CBGA-Hunig's base amine salt (87.9% yield) and was analysed by HPLC (FIG. 27). The sample that was crystallized without the heptane spike yielded and 0.681 g of a white crystalline CBGA-Hunig's base amine salt (82.3% yield) and was analysed by HPLC (FIG. 28).

It should be noted that FIGS. 24 to 28 for Example 9 show a very small peak at a retention time of about 9 min that is identified as “THC-A”. This peak is not due to the presence of THC-A but instead, is an unknown anomaly associated with the filters in the syringes used to prepare the samples for HPLC analyses (FIGS. 16, 17).

### Example 10

This example assessed methods for extracting, recovery, and purification of crude CBGA-amine salts from a dried-down complex crude mixture of phytochemicals recovered from Cannabis biomass wherein 1-butanol was the organic solvent wherein to the dried-down complex crude mixture was resolubilized and standardized prior to addition of an amine to thereby precipitate a crude CBGA-amine salt.

A sample of HURV19PAN hemp kief was extracted with cold ethyl acetate (−20° C.) after which the crude complex ethyl acetate extract solution was recovered following the method disclosed in Example 6. A dried resin comprising a complex mixture of compounds was produced by evaporation of the ethyl acetate following the method disclosed in Example 6 after which, the resin was resolubilized in 1-butanol to form a standardized crude extract solution.

The CBGA content in the standardized crude extract 1-butanol solution was quantified by removing a 10-uL sample of the standardized crude extract solution, separating the 1-butanol under vacuum, dissolving the resulting resin in 1 mL of HPLC-grade methanol and further preparing a 10× dilution of the sample in HPLC-grade methanol. The 10× diluted sample was then analyzed by HPLC and the standardized crude extract solution in 2-propanol was determined to contain 79.106 mg/mL CBGA (FIG. 29).

Two 5-mL aliquots of the standardized crude extract solution in 1-butanol were heated to 30° C. after which, a 3:1 molar ratio of N,N-diisopropylethylamine (Hunig's base; 0.573 mL)] was added dropwise to each aliquot while mixing with a magnetic stir bar. Then, heptane was added in (i) a 1:1 ratio vol./vol. or (ii) a 1.75:1 ratio vol./vol. of heptane to 1-butanol standardized extract to thereby cause precipitation of a crude CBGA-Hunig's base amine salt. The solid CBGA-Hunig's base amine salt was separated from the liquid phase by vacuum filtration, washed with cold heptane, vacuum filtered, and dried. HPLC analyses of the precipitated salts confirmed that the yield in the aliquot receiving a heptane spike at a 1:1 ratio vol./vol. was 79.46% (FIG. 30), whereas the yield in the aliquot receiving a heptane spike at a 1.75:1 ratio vol./vol. was 85.66% (FIG. 31).

It should be noted that FIGS. 29 to 31 for Example 10 show a very small peak at a retention time of about 9 min that is identified as “THC-A. This peak is not due to the presence of THC-A but instead, is an unknown anomaly associated with the filters in the syringes used to prepare the samples for HPLC analyses (FIGS. 16, 17).

### Example 11

Two studies were performed to assess the potential of twelve selected amine compounds from a range of amines, for reliable and routine precipitation of cannabidivarinic acid-amine salts (CBDVA-amine salts).

The first study assessed the potential of each of the twelve amines listed in Table 4 for its potential to crystallize (i.e., precipitate) CBDVA from selected organic solvent solutions by dropwise addition of the amine into a solubilized CBDVA solution to provide an equimolar quantity (1:1 ratio) of the amine. For each assay, 25 mg of pure CBDVA was dissolved in 0.5 mL of ethyl acetate. Each of the amines was dissolved in 0.5 mL ethyl acetate and then mixed with the dissolved CBDVA in ethyl acetate. Then, 0.5 mL heptane was added to each mixture as the antisolvent and the mixture was vigorously mixed.

Seven of the twelve amines assessed in this study precipitated CBDVA as an amine salt from its ethyl acetate solutions (Table 4). Four amines precipitated CBDVA-amine salts during vigorous mixing at ambient room temperature (about 20° C.):
1. CBDVA-triethylamine salt (precipitation occurred immediately after addition of the amine prior to addition of the antisolvent),
2. CBDVA-Hunig's base amine salt (precipitation occurred immediately after addition of the amine prior to addition of the antisolvent),
3. CBDVA-DBN amine salt (precipitation occurred immediately after addition of the amine prior to addition of the antisolvent), and
4. CBDVA-DMEA amine salt (precipitation occurred immediately after addition of the amine prior to addition of the antisolvent).

Three more amines precipitated CBDVA-amine salts from an ethyl acetate solution after addition of the heptane antisolvent and lowering the temperature of reaction mixture to about 0° C.:
5. CBDVA-cyclohexylisopropylamine salt,
6. CBDVA-TMEDA amine salt, and
7. CBDVA-methylpiperazine amine salt.

TABLE 4

| | | Crystals | Precipitation | |
|---|---|---|---|---|
| | Amine | formed | temp. (° C.) | |
| | Primary amines | | | |
| 1 | cyclohexylamine | no | | |
| | Secondary amines | | | |
| 2 | diethylamine | no | | |
| 3 | morpholine | no | | |
| | Tertiary amines | | | |
| 4 | triethylamine | YES | 20° | C. |
| 5 | tributylamine | no | | |
| 6 | N,N-diisopropylethylamine (Hunig's base) | YES | 20° | C. |
| | Amino alcohols | | | |
| 7 | dimethylethanolamine (DMEA) | YES | 20° | C. |
| | Diamines | | | |
| 8 | 1,4-diazabicyclo[2.2.2]octane (DABCO) | no | | |
| 9 | tetramethylethylenediamine (TMEDA) | YES | 0° | C. |
| 10 | N-methylpiperazine | YES | 0° | C. |
| | Highly basic amines | | | |
| 11 | 1,5-diazabicyclo(4.3.0)non-5-ene (DBN) | YES | 20° | C. |
| | Other | | | |
| 12 | N-cyclohexylisopropylamine | YES | 0° | C. |

The second study assessed the precipitation of amine salts from butanol solutions containing CBDVA. It was determined that triethylamine, Hunig's base, and dimethylethanolamine precipitated CBDVA from butanol solutions into which CBDVA had been dissolved.

Each of the CBDVA-amine salts was assessed by differential scanning calorimetry (DSC) analysis with a TA Discovery DSC instrument equipped with an autosampler, a reference-loaded furnace, and hermetically sealed aluminum crucibles. 1-4 mg of each CBDVA-amine salts were placed into separate DSC crucibles, weighed, and then placed into the autosampler tray and analyzed.

Each of the CBDVA-amine salts was assessed by qualitative HPLC analysis in reference to a standards solution containing CBDVA, CBDA, CBGA, THCVA, THCA (FIG. 32). As the amine counterions were not detectable due to lack of chromophores, the dominant spectral signal which was recorded, comes from the cannabinoid acid unit of the salt. Cannabinoid acid reference standard solutions were prepared in HPLC-grade methanol containing 0.05% formic acid vol./vol., for each of CBDVA, CBDA, CBGA, THCVA, THCA wherein the final concentrations were 0.05 mg/mL (wt./vol.). 200 μL of each standard solution were combined into a 1-mL solution. Each of the individual standards was analyzed by injection into an Agilent 1220 HPLC equipped with an InfinityLab Poroshell 120 EC-C18 column and Agilent OpenLAB v2.0 software. The HPLC chromatogram peaks for the individual cannabinoids directly corresponded with their peaks in the HPLC chromatogram for the combined standards solution (FIG. 32). The samples for HPLC analysis of each of the CBDVA-amine salts were prepared as subsamples from their corresponding sample solutions prepared for the ¹H NMR analyses described below. 100 μL of each CBGVA-amine salt were brought to final volumes of 1 mL with HPLC-grade methanol, and then sonicated for 5 min, The samples were then filtered through 0.2 μm filters, and subsamples transferred into HPLC vials (discarding the initial few drops) and then injected into the HPLC column.

About 50 μg of each CBDVA-amine salt were added to 1-mL volumes of CDCl₃. A 0.7-mL subsample was characterized by taking their ¹H NMR spectra at 400 MHz. The remaining aliquots were used for HPLC analyses as described above.

The DSC thermogram produced with CBDVA is shown in FIG. 33. CBDVA has the chemical structure shown in (11).

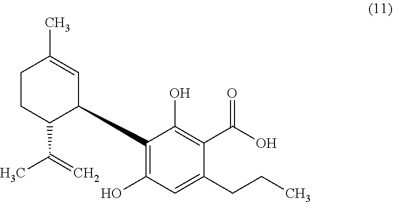

### Salt 1. CBDVA-triethylamine Salt (TEA)

The DSC thermogram produced with CBDVA-TEA amine salt is shown in FIG. 34. The HPLC analysis of the CBDVA-TEA amine salt is shown in FIG. 35. The ¹HNMR spectra in CDCl₃ and recording at 400 MHz are shown in FIG. 36 and confirmed the chemical structure shown in (12).

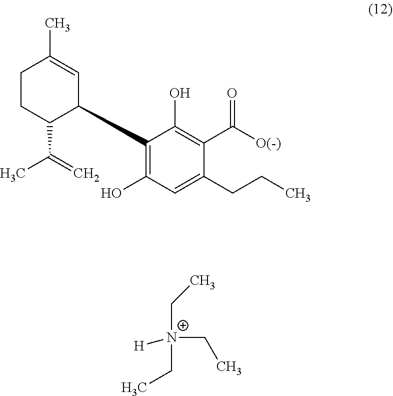

### Salt 2. CBDVA-14,N-diisopropylethylamine Salt (Hunig's Base)

The DSC thermogram produced with CBDVA-Hunig's base amine salt is shown in FIG. 37. The HPLC analysis of the CBDVA-Hunig's base amine salt is shown in FIG. 38. The ¹HNMR spectra in CDCl₃ and recording at 400 MHz are shown in FIG. 39 and confirmed the chemical structure shown in (13).

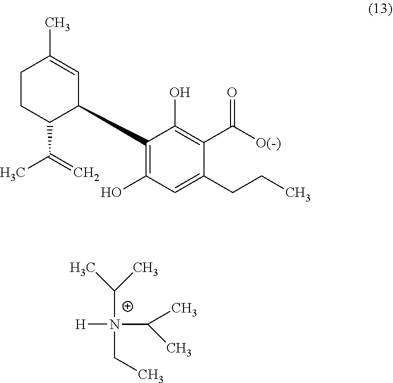

### Salt 3. CBDVA-1,5-diazabicyclo(4.3.0)non-5-ene Salt (DBN)

The DSC thermogram produced with CBDVA-DBN amine salt is shown in FIG. 40. The HPLC analysis of the CBDVA-DBN amine salt is shown in FIG. 41. The ¹HNMR spectra in CDCl₃ and recording at 400 MHz are shown in FIG. 42 and confirmed the chemical structure shown in (14).

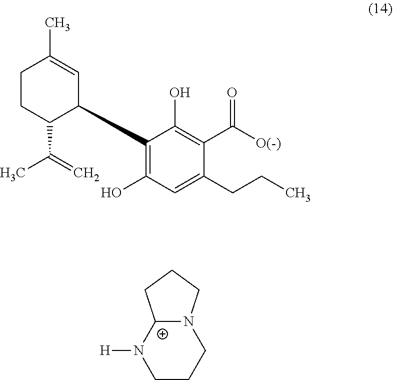

### Salt 4. CBDVA-dimethylethanolamine Salt (DMEA)

The DSC thermogram produced with CBDVA-DMEA amine salt is shown in FIG. 43. The HPLC analysis of the CBDVA-DMEA amine salt is shown in FIG. 44. The ¹HNMR spectra in CDCl₃ and recording at 400 MHz are shown in FIG. 45 and confirmed the chemical structure shown in (15).

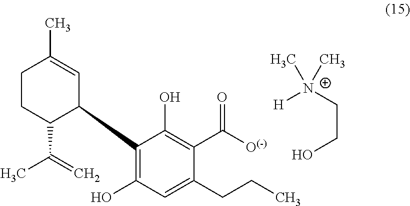

### Salt 5. CBDVA-cyclohexylisopropylamine Salt (CHIPA)

The ¹HNMR spectra for the CBDVA-DMEA salt in CDCl₃ and recording at 400 MHz are shown in FIG. 46 and confirmed the chemical structure shown in (16).

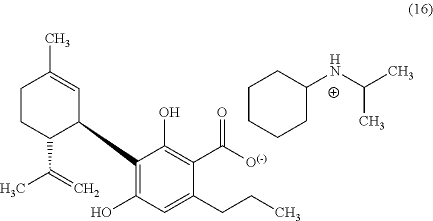

### Salt 6. CBDVA-tetramethylethylenediamine Salt (TMEDA)

The DSC thermogram produced with CBDVA-TMEDA amine salt is shown in FIG. 47. The HPLC analysis of the CBDVA-TMEDA amine salt is shown in FIG. 48. The ¹HNMR spectra in CDCl₃ and recording at 400 MHz are shown in FIG. 49 and confirmed the chemical structure shown in (17).

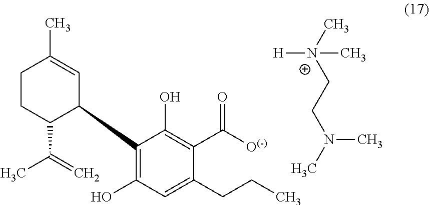

### Salt 7. CBDVA-methylpiperazine amine Salt

The HPLC analysis of the CBDVA-metylpiperidine amine salt is shown in FIG. 50. The ¹HNMR spectra for the CBDVA-methylpiperazine amine salt are shown in FIG. 51 and confirmed the chemical structure shown in (18).

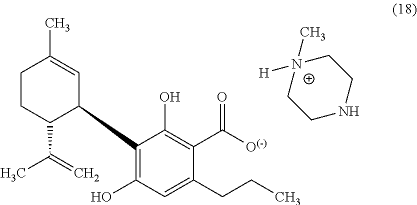

### Example 12

This study assessed the potential of twelve selected amine compounds from a range of amines, for reliable and routine precipitation of Δ9-tetrahydrocannabivirinic acid-amine salts (THCVA-amine salts) from ethanol and butanol solutions

The first study assessed the potential of each of the twelve amines listed in Table 5 for its potential to crystallize (i.e., precipitate) THCVA from selected organic solvent solutions by dropwise addition of the amine into a solubilized THCVA solution to provide an equimolar quantity (1:1 ratio) of the amine. For each assay, 25 mg of pure THCVA was dissolved into 0.5 mL of ethanol or butanol. Each of the amines was dissolved in 0.5 mL ethyl acetate and then mixed with the dissolved THCVA in ethanol or butanol. Then, 0.5 mL heptane was added to each mixture as the antisolvent and the mixture was vigorously mixed.

TABLE 5

| | | Crystals | Precipitation |
|---|---|---|---|
| | Amine | formed | temp. (° C.) |
| | Primary amines | | |
| 1 | cyclohexylamine | no | |
| 2 | butylamine | no | |
| | Secondary amines | | |
| 3 | diethylamine | no | |
| 4 | morpholine | no | |
| | Tertiary amines | | |
| 5 | triethylamine | no | |
| 6 | tributylamine | no | |
| 7 | N,N-diisopropylethylamine (Hunig's base) | no | |
| | Amino alcohols | | |
| 8 | dimethylethanolamine (DMEA) | YES | 0° C. |
| | Amino ethers | | |
| 9 | morpoholine | no | |
| | Highly basic amines | | |
| 10 | 1,5-diazabicyclo(4.3.0)non-5-ene (DBN) | YES | 0° C. |
| | Other | | |
| 11 | N-cyclohexylisopropylamine | YES | 0° C. |

Three amines precipitated THCVA-amine salts from THCVA solubilized in ethanol with a 1:1 vol./vol. spike of heptane with mixing at about 0° C.; (i) dimethylethanolamine, 1,5-diazabicyclo(4.3.0)non-5-ene, and (iii) N-cyclohexylisopropylamine. The same three amines also precipitated THCVA-amine salts from THCVA solubilized in 1-butanol with a 1:1 vol./vol. spike of heptane with mixing at about 0° C.

Each of the THCVA-amine salts was assessed by differential scanning calorimetry (DSC) analysis in reference to THCVA, and was characterized by taking their ¹H NMR spectra in CDCl₃ and recording at 400 MHz.

Each of the THCVA-amine salts was assessed by qualitative HPLC analysis in reference to a standards solution containing CBDVA, CBDA, CBGA, THCVA, THCA (FIG. 32). The samples for HPLC analysis of each of the THCVA-amine salts were prepared as subsamples from their corresponding sample solutions prepared for the ¹H NMR analyses described below. 100 μL of each THCVA-amine salt were brought to final volumes of 1 mL with HPLC-grade methanol, and then sonicated for 5 min, The samples were then filtered through 0.2 μm filters, and subsamples transferred into HPLC vials (discarding the initial few drops) and then injected into the HPLC column.

About 50 μg of each THCVA-amine salt were added to 1-mL volumes of CDCl₃. A 0.7-mL subsample was characterized by taking their ¹H NMR spectra at 400 MHz. The remaining aliquots were used for HPLC analyses as described above.

The DSC thermogram produced with THCVA is shown in FIG. 52. THCVA has the chemical structure shown in (19).

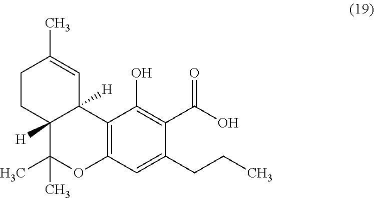 [SMILES: CCCc1cc2c(c(O)c1C(=O)O)[C@@H]1C=C(C)CC[C@H]1C(C)(C)O2]

### Salt 1. THCVA-dimethylethanolamine Salt (DMEA)

The DSC thermogram produced with THCVA-DMEA amine salt is shown in FIG. 53. The HPLC analysis of the THCVA-DMEA amine salt is shown in FIG. 54. The ¹HNMR spectra in CDCl₃ and recording at 400 MHz are shown in FIG. 55 and confirmed the chemical structure shown in (20).

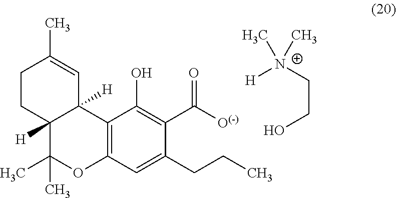

### Salt 2. THCVA-1,5-diazabicyclo(4.3.0)non-5-ene Salt (DBN)

The HPLC analysis of the THCVA-DBN amine salt is shown in FIG. 56. The ¹HNMR spectra for the THCVA-DBN amine salt are shown in FIG. 57 and confirmed the chemical structure shown in (21).

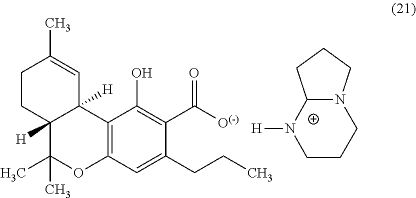

### Salt 3. THCVA-cyclohexylisopropylamine Salt (CHIPA)

The DSC thermogram produced with THCVA-CHIPA amine salt is shown in FIG. 58. The HPLC analysis of the THCVA-DBN amine salt is shown in FIG. 59. The ¹HNMR spectra in CDCl₃ and recording at 400 MHz are shown in FIG. 60 and confirmed the chemical structure shown in (22).

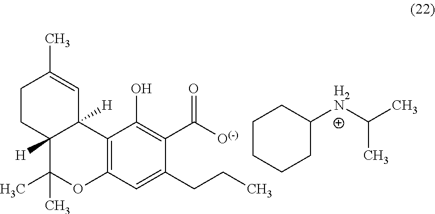

### Example 13

A screening study was performed to assess the potential of thirteen selected amine compounds from a range of amines, for reliable and routine precipitation of cannabichromevarinic acid (CBCVA) from complex mixtures. These were:
(i) diisopropylamine (a secondary amine),
(ii) isopropylcyclohexylamine (a secondary amine),
(iii) 2,2,6,6-tetramethylpiperidine (a secondary amine),
(iv) dicyclohexylamine (a secondary amine),
(v) triethylamine (a tertiary amine),
(vi) N,N-diisopropylethylamine (Hunig's base; a tertiary amine),
(vii) methyldicyclohexylamine (a tertiary amine),
(viii) dimethylethanolamine (DMEA; an amino alcohol),
(ix) piperidineethanol (an amino alcohol),
(x) 1,8-diazabicyclo[5.4.0]undec-7-ene (DBU; a diamine)
(xi) 1,5-diazabicyclo[4.3.0]non-5-ene (DBN; a diamine)
(xii) 1,4-diazabicyclo[2.2.2]octane (DABCO; a diamine),
(xiii) dimethylaminopyridine (DMAP; a diamine), all of which have been shown previously to precipitate one or more of THCA, CBDA, and CBGA.

Each of these salts listed in Table 6 was screened as follows. First, a solution of CBCVA was prepared in hexane (40 mg/mL) after which, 50 mg of a selected amine solution was added after which, the solvents were evaporated to yield one of an oil or a solid (Table 6).

Seven of the thirteen amines precipitated solid CBCVA-amine salts:
(i) isopropylcyclohexylamine formed 67 mg of amine salt from 80 mg of CBCVA,
(ii) 2,2,6,6-tetramethylpiperidine formed 40 mg of amine salt from 80 mg of CBCVA,
(iii) dicyclohexylamine formed 67 mg of amine salt from 80 mg of CBCVA,
(iv) Hunig's base formed 18 mg of amine salt from 40 mg of CBCVA,
(v) DBN formed 67 mg of amine salt from 80 mg of CBCVA,
(vi) DABCO formed 60 mg of amine salt from 80 mg of CBCVA,
(vii) DMAP formed 68 mg of amine salt from 80 mg of CBCVA.

TABLE 6
Summary of salt formation of CBCVA with various amines.

| | | Crystals | Melting |
|---|---|---|---|
| | Amines | formed | temp. (° C.) |
| | Secondary amines | | |
| 1 | diisopropylamine | no | |
| 2 | isopropylcyclohexylamine | YES | 91-92 |
| 3 | 2,2,6,6-tetramethylpiperidine | YES | 136-139 |
| 4 | dicyclohexylamine | YES | 159-160 |
| | Tertiary amines | | |
| 5 | triethylamine | no | |
| 6 | N,N-diisopropylethylamine (Hunig's base) | YES | |
| 7 | N-methyldicyclohexylamine | no | |
| | Amino alcohols | | |
| 8 | dimethylethanolamine (DMEA) | no | |
| 9 | piperidineethanol | no | |
| | Diamines | | |
| 10 | 1,8-diazabicyclo[5.4.0]undec-7-ene (DBU) | no | |
| 11 | 1,5-diazabicyclo[4.3.0]non-5-ene (DBN) | YES | 77-78 |
| 12 | 1,4-diazabicyclo[2.2.2]octane (DABCO) | YES | 125-127 |
| 13 | dimethylaminopyridine (DMAP) | YES | 121-122 |

The seven CBCVA-amine salts produced were characterized by taking their ¹H NMR spectra in CDCl₃ and recording at 400 MHz. Each of the CBCVA-amine salts showed the expected peaks due to the ammonium ion in addition to all the peaks comprising the CBCVA acid unit. The integration of the peaks was consistent with a 1:1 ratio of ammonium ion vs CBCVA carboxylate (23).

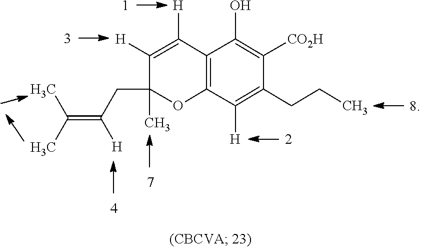

The key peaks of the carboxylate portion (23) and key peaks due to the ammonium ion which do not overlap with the CBCV carboxylate peaks are reported. The eight carboxylate peaks starting with the most deshielded peak due to H1 and ending with peak of the terminal methyl group, H8, are reported in bold in the spectroscopic data for each of the salts. These peaks are found at 6.72 (d, J=10.0 Hz, 1H), 6.22 (s, 1H), 5.46 (d, J=10.0 Hz), 1H), 5.07 (m, 1H), 1.63 (d, I=0.8 Hz, 3H), 1.55 (s, 3H), 1.39 (s, 3H) and) 0.95 (t, J=7.6 Hz, 3H) in CBCV-carboxylate. The peak assignment and the integration of the relevant ammonium ion peaks are also given.

The ¹³C spectra of the salts all show the expected number of unique carbon signals.

### Salt 1. CBCVA-isopropylcyclohexylamine Salt

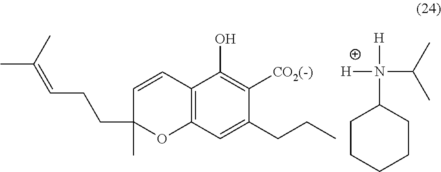

¹H NMR (400 MHz, CDCl₃). δ (ppm) values for key peaks due to CBCVA. 6.76 (d. J=10 Hz, 1H), 6.07 (s, 1H), 5.37 (d, J=10 Hz, 1H), 5.06 (t, J=6.8 Hz, 2H), 1.63 (s, 3H), 1.55 (s, 3H), 1.34 (s, 3H), 0.90 (t, 3H).

Key peaks due to the ammonium ion, δ (ppm): 3.98 (sept., 2H), 3.06 (q, J=7.6 Hz, 2H), 1.38 (d, J=6.8 Hz), 12H).

¹³C NMR (CDCl₃) δ (ppm): 174.6, 160.0, 154.9, 147.8, 131.5, 125.1, 124.4, 118.2, 110.3, 108.5, 106.9, 78.4, 52.9, 41.5, 41.3, 38.0, 26.6, 25.7, 24.9, 22.7, 18.0, 17.6, 14.4, 11.8.

### Salt 2. CBCVA-2,2,6,6-tetramethylpiperidine Salt

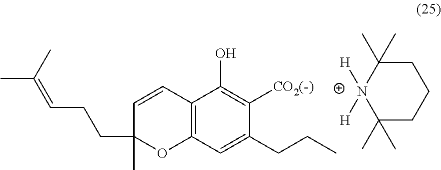

¹H NMR (400 MHz, CDCl₃). δ (ppm) values for key peaks due to CBCVA: 6.76 (d. J=10 Hz, 1H), 6.11 (s, 1H), 5.40 (d, J=10 Hz, 1H), 5.08 (m, 1H), 3.06 (m, 2H), 1.64 (s, 3H), 1.56 (s, 3H), 1.36 (s, 3H), 0.91 (t, 3H).

Key peak due to the ammonium ion, δ (ppm): 1.45 (s, 12H).

¹³C NMR (CDCL₃) δ (ppm): 174.6, 160.0, 155.1, 148.0, 131.5, 125.3, 124.3, 118.0, 110.5, 109.0, 106.8, 78.6, 56.0, 41.4, 37.9, 34.9, 31.6, 27.4, 26.7, 25.7, 24.8, 22.8, 17.6, 16.6, 14.4.

### Salt 3: CBCVA-N,N-dicyclohexylamine Amine Salt

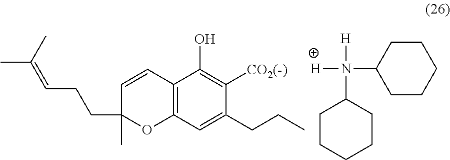

¹H NMR (400 MHz, CDCl₃). δ (ppm) values for key peaks due to CBCVA: 6.74 (d. J=10 Hz, 1H), 6.12 (s, 1H), 5.41 (d, J=10 Hz, 1H), 5.08 (m, 1H), 1.64 (s, 3H), 1.56 (s, 3H), 1.37 (s, 3H), 0.93 (t, 3H).

Key peaks due to the ammonium ion, δ (ppm): 3.36 (s, 1H), 2.95 (m, 1H).

¹³C NMR (CDCl₃) δ (ppm): 174.1, 159.4, 155.2, 147.8, 131.6, 125.5, 124.3, 117.9, 110.4, 109.3, 106.9, 78.6, 52.8, 41.4, 38.1, 28.9, 26.8, 25.7, 25.1, 24.9, 24.8, 22.7, 17.6, 14.5.

### Salt 4: CBCVA-N,N-diisopropylethylamine Salt (Hunig's Base)

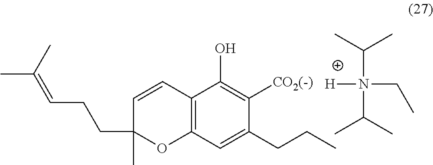

¹H NMR (400 MHz, CDCl₃). δ (ppm) values for key peaks due to CBCVA: 6.76 (d. J=10 Hz, 1H), 6.07 (s, 1H), 5.37 (d, J=10 Hz, 1H), 5.06 (t, J=6.8 Hz, 2H), 3.06 (m, 2H), 1.63 (s, 3H), 1.55 (s, 3H), 1.34 (s, 3H), 0.90 (t, 3H).

Key peak due to the ammonium ion, δ (ppm): 3.98 (sept., 2H), 3.06 (q, J=7.6 Hz, 2H) 1.38 (d, J=6.8 Hz, 12H).

¹³C NMR (CDCL₃) δ (ppm): 174.6, 160.0, 154.9, 147.8, 131.5, 125.1, 124.4, 118.2, 109.3, 108.9, 106.9, 78.4, 52.9, 41.5, 41.3, 38.0, 26.6, 25.7, 24.9, 22.7, 18.0, 17.6, 14.4, 11.8.

### Salt 5: CBCVA 1,5-diazabicyclo [4.3.0]non-5-ene (DBN)

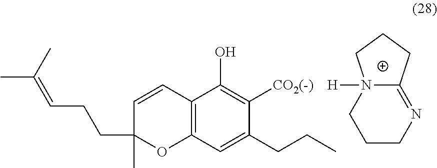

¹H NMR (400 MHz, CDCl₃). δ (ppm) values for key peaks due to CBCVA: 6.71 (d. J=10 Hz, 1H), 6.04 (s, 1H) 5.36 (d, J=10 Hz, 1H) 5.05 (m, 1H) 1.64 (s, 3H), 1.53 (s, 3H), 1.33 (s, 3H), 0.90 (t, 3H).

Key peak due to the ammonium ion, δ (ppm): 3.43 (m, 4H), 3.01 (m, 4H).

¹³C NMR (CDCl₃) δ (ppm): 174.0, 159.6, 155.1, 148.0, 131.5, 125.3, 124.3, 118.0, 110.5, 108.9, 106.8, 78.6, 56.0, 41.4, 37.9.9, 34.9, 27.8, 26.7, 25.7, 24.8, 22.8, 17.7, 16.6, 14.2.

### Salt 6. CBCVA-1,4-diazabicyclo[2.2.2]octane (DABCO)

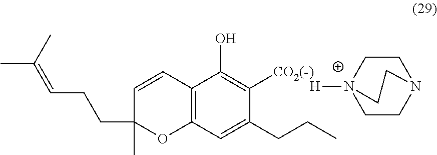

¹H NMR (400 MHz, CDCl₃). δ (ppm) values for key peaks due to CBCVA: 6.74 (d. J=10 Hz, 1H), 6.01 (s, 1H), 5.39 (d, J=10 Hz, 1H), 5.06 (t, J=6.8 Hz, 1H), 2.93 (m, 2H), 1.62 (s, 3H), 1.53 (s, 3H), 1.34 (s, 3H), 0.90 (t, 3H).

Key peak due to the ammonium ion, δ (ppm): 3.05 (s, 12H).

### Salt 7. CBCVA-4-dimethylaminopyridine (DMAP)

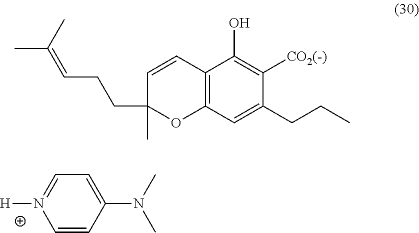

¹H NMR (400 MHz, CDCl₃). δ (ppm) values for key peaks due to CBCVA: 6.76 (d. J=10 Hz, 1H), 6.10 (s, 1H) 5.39 (d, J=10 Hz, 1H) 5.07 (m, 1H), 1.65 (s, 3H), 1.55 (s, 3H), 1.36 (s, 3H), 0.92 (t, 3H).

Key peaks due to the ammonium ion, δ (ppm) 8.21 (d, J=6.0 Hz, 2H), 6.58 (d, J=6.0 Hz, 2H), 3.06 (s, 6H).

¹³C NMR (CDCl₃) δ (ppm) 175.7, 160.1, 156.8, 155.3 148.2, 141.3, 131.5, 125.2, 124.3, 118.1, 109.0, 106.8, 106.5, 78.5, 41.4, 39.7, 38.0, 26.7, 25.7, 24.8, 22.7, 17.6, 14.5.

### Example 14

A screening study was performed to assess the potential of seven selected amine compounds from a range of amines, for reliable and routine precipitation of cannabichromic acid (CBCA) from complex mixtures. These were:
(i) ethyldiisopropylamine (a secondary amine),
(ii) 2,2,6,6-tetramethylpiperidine (a secondary amine),
(iii) dicyclohexylamine (a secondary amine),
(iv) triethylamine (a tertiary amine),
(v) 1,5-diazabicyclo[4.3.0]non-5-ene (DBN; a diamine),
(vi) dimethylaminopyridine (DMAP; a diamine),
(vii) 1-methylpiperazine, all of which have been shown previously to precipitate one or more of THCA, CBDA, and CBCA.

Each of these salts was screened as follows. First a solution of pure CBCA was dissolved in DCM (50 mg/mL), followed by the addition of 50 mg of a selected amine, followed by hexane. The solvents were evaporated to yield one of an oil or a solid (Table 7).

TABLE 7
Summary of salt formation of CBCA with various amines.

| | | Crystals | Melting |
|---|---|---|---|
| | Amines | formed | temp. (° C.) |
| | Secondary amines | | |
| 1 | ethyldiisopropylamine | no | |
| 2 | 2,2,6,6-tetramethylpiperidine | no | |
| 3 | dicyclohexylamine | no | |
| | Tertiary amines | | |
| 4 | triethylamine | no | |
| | Diamines | no | |
| 5 | 1,5-diazabicyclo[4.3.0]non-5-ene (DBN) | YES | |
| 6 | 4-dimethylaminopyridine (DMAP) | YES | 86-88 |
| 7 | 1-methylpiperazine | no | |

Two of the seven amines precipitated solid CBCA-amine salts:
(i) DBN formed 75 mg of amine salt from 75 mg of CBCA,
(ii) 4-dimethylaminopyridine formed 33 mg of amine salt from 40 mg of CBCA.

The two CBCA-amine salts produced were characterized by taking their ¹H NMR spectra in CDCl₃ and recording at 400 MHz. Each of the CBCA-amine salts showed the expected peaks due to the ammonium ion in addition to all the peaks comprising the CBCA acid unit. The integration of the peaks was consistent with a 1:1 ratio of ammonium ion vs CBCA carboxylate (31).

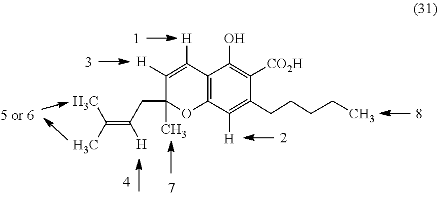

The key peaks of the carboxylate portion (31) and key peaks due to the ammonium ion which do not overlap with the CBC carboxylate peaks are reported. The eight carboxylate peaks are listed starting with the most deshielded peak due to H1 and ending with peak of the terminal methyl group, H8, are reported in bold in the spectroscopic data for each of the salts. These peaks are found at 6.72 (d, J=10.0 Hz, 1H), 6.23 (s, 1H), 5.46 (d, J=10.0 Hz), 1H), 5.07 (m, 1H), 1.64 (s, 3H), 1.56 (s, 3H), 1.39 (s, 3H) and 0.89 (t, J=7.6 Hz, 3H) in CBC-Acid. The peak assignment and the integration of the relevant ammonium ion peaks are also given.

The ¹³C spectra of the salts all show the expected number of unique carbon signals.

### Salt 1. CBCA-1,5-diazabicyclo[4.3.0]non-5-ene Salt (DBN)

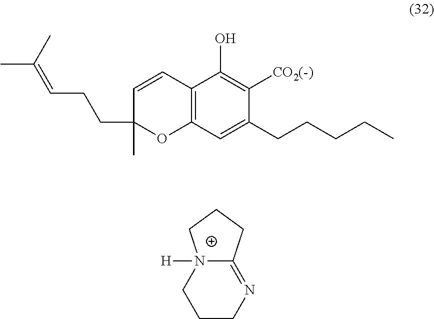

¹H NMR (400 MHz, CDCl₃). δ (ppm) values for key peaks due to CBCA: 6.33 (d, J=10 Hz, 1H), 6.05 (s, 1H), 5.36 (d, J=10.0 Hz, 1H), 5.06 (m, 1H), 1.62 (s, 3H), 1.554 (s, 3H) 1.33 (s, 3H), 0.84 (t, 3H).

Key peaks due to the ammonium ion, δ (ppm): 3.2 (broad s, 4H) 3.05 (m, 4H).

¹³C NMR (CDCl₃) δ (ppm): 174.0, 164.4, 160, 154.8 148.0, 131.5, 125.3, 124.0, 118.2, 110.5, 108.5, 106.7, 78.3, 53.0, 42.4, 41.3, 37.8, 31.6, 27.8, 29.8, 26.6, 25.7, 24.8, 22.7,18.7, 17.6, 14.5.

### Salt 2. CBCA-4-dimethylaminopyridine Salt (DMAP)

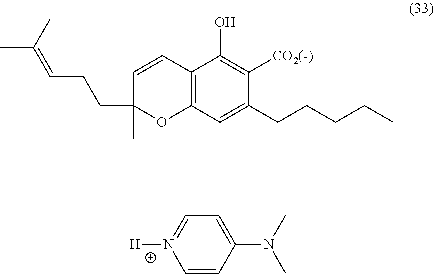

¹H NMR (400 MHz, CDCl₃). δ (ppm) values for key peaks due to CBCA: 6.75 (d, J=10 Hz, 1H), 6.10 (s, 1H), 5.38 (d, J=10.0 Hz, 1H), 5.06 (m, 1H), 1.62 (s, 3H), 1.54 (s, 3H) 1.35 (s, 3H), 0.84 (t, 3H).

Key peaks due to the ammonium ion, δ (ppm): 8.26 (d, J=5.2 Hz, 2H), 6.62 (d, J=5. 2Hz. 2H), 3.11 (s, 6H).

¹³C NMR (CDCl₃) δ (ppm): 175.6, 160.1, 156.7, 155.3 148.5, 141.3, 131.6, 125.2, 124.3, 118.1, 108.9, 106.8, 106.5, 78.5, 52.7, 41.4, 39.8, 35.9, 32.2, 31.5, 26.7, 25.7, 22.7, 17.7, 14.

1-99. (canceled)

100. A method for separating, recovering, and purifying a cannabigerolic acid-amine salt (CBGA-amine salt) or a Δ9-tetrahydrocannabivaric acid-amine salt (THCVA-amine salt) or a cannabidivarinic acid-amine salt (CBDVA-amine salt) or a cannabichromevarinic acid-amine salt (CBCVA-amine salt) or a cannabichromenic acid-amine salt (CBCA-amine salt from an organic solvent solution comprising a mixture of cannabinoids, said method comprising:
(i) providing a crude complex extract or mixture containing therein a complex mixture of metabolites and cannabinoids recovered from a Cannabis biomass or from cultured microbial fermentation systems, with an extractant solvent selected from one of methanol, ethanol, propanol, isopropanol, butanol, heptane, hexane, propane, butane, ethyl acetate, acetone, dichloromethane, 1,4-dioxane, tetrahydrofuran, acetonitrile, toluene, methyl tert-butyl ether, supercritical CO₂, subcritical CO₂, or recovered by microfiltration;
(ii) removing the extractant solvent from the crude extract or mixture and drying the desolventized crude extract or mixture to thereby produce a concentrated complex extract;
(iii) assaying the concentrated crude extract or mixture to determine a first concentration of CBGA or THCVA or CBDVA or CBCVA or CBCA therein;
(iv) adding a volume of a first organic solvent selected from ethyl acetate, ethanol, isopropanol, propanol, butanol, hexane, heptane and dichloromethane, to the concentrated crude extract and commingling therewith to adjust the first CBGA or THCVA or CBDVA or CBCVA or CBCA concentration to a target concentration value selected from a range of target concentrations, thereby producing a solvent-solubilized complex extract solution or complex mixture solution;
(v) adding a selected amine to the solvent-solubilized complex extract solution or complex mixture solution, and commingling therewith to precipitate therefrom a CBGA-amine salt or THCVA-amine salt or CBDVA-amine salt or CBCVA-amine salt or CBCA-amine salt;
(vi) washing the precipitated CBGA-amine salt or THCVA-amine salt or CBDVA-amine salt or CBCVA-amine salt or CBCA-amine salt at least once with said first organic solvent and then drying the washed CBGA-amine salt or THCVA-amine salt or CBDVA-amine salt or CBCVA-amine salt or CBCA-amine salt;
(vii) dissolving the washed and dried CBGA-amine salt or THCVA-amine salt or CBDVA-amine salt or CBCVA-amine salt or CBCA-amine salt in a second organic solvent selected from ethanol, denatured ethanol, ethyl acetate, and dichloromethane, and commingling therewith;
(viii) adding a volume of an antisolvent selected from pentane, hexane, heptane, petroleum ethers, and water, to the dissolved CBGA-amine salt or THCVA-amine salt or CBDVA-amine salt or CBCVA-amine salt or CBCA-amine salt and commingling therewith to thereby recrystallize the CBGA-amine salt or THCVA-amine salt or CBDVA-amine salt or CBCVA-amine salt or CBCA-amine salt; and
(ix) washing the recrystallized CBGA-amine salt or THCVA-amine salt or CBDVA-amine salt or CBCVA-amine salt or CBCA-amine salt at least once with said selected antisolvent to produce a purified CBGA-amine salt or THCVA-amine salt or CBDVA-amine salt or CBCVA-amine salt or CBCA-amine salt, and then drying the purified CBGA-amine salt or THCVA-amine salt or CBDVA-amine salt or CBCVA-amine salt or CBCA-amine salt.

101. The method according to claim 100, additionally comprising the steps of:
(x) re-solubilizing the purified CBGA-amine salt or THCVA-amine salt or CBDVA-amine salt or CBCVA-amine salt or CBCA-amine salt from step (ix) in a third organic solvent selected from ethyl acetate, heptane, hexane, pentane, butanol, and dichloromethane, then
(xi) acidifying the solubilized purified CBGA-amine salt or THCVA-amine salt or CBDVA-amine salt or a CBCVA-amine salt or CBCA-amine salt with a mineral acid solution selected from HCl and H₂SO₄ to partition therefrom an organic layer containing the highly purified CBGA or THCVA or CBDVA or CBCVA or CBCA, and an aqueous layer containing the amine as its hydrochloride;
(xii) separating the aqueous layer from the organic layer containing the highly purified CBGA or THCVA or CBDVA or CBCVA or CBCA; and
(xiii) desolventizing the CBGA or THCVA or CBDVA or CBCVA or CBCA to produce highly purified CBGA or THCVA or CBDVA or CBCVA or CBCA.

102. The method according to claim 100, additionally comprising the steps of:
(xiv) decarboxylating the purified CBGA-amine salt or THCVA-amine salt or CBDVA-amine salt or CBCVA-amine salt or CBCA-amine salt by suspending the purified CBGA-amine salt or CBDVA-amine salt or THCVA-amine salt or CBCVA-amine salt or CBCA-amine salt in a selected volume of a sodium carbonate solution and then heating the sodium carbonate solution at about 100° C. under constant mixing to thereby produce therein a mixture of CBG or CBDV or THCV or CBCV or CBC and the amine, to produce an oil containing CBG or CBCV or CBDV or THCV or CBC and amine;
(xv) solubilizing the oil containing decarboxylated CBG or CBCV or CBDV or THCV or CBC and amine in a fourth organic solvent selected from ethyl acetate, heptane, hexane, pentane, butanol, and dichloromethane, to thereby partition therefrom an organic layer containing a highly purified CBG or CBCV or CBDV or THCV or CBC oil and separated amine organic layer, and an aqueous layer;
(xvi) separating the organic layer containing the highly purified CBG or CBCV or CBDV or THCV or CBC and amine from the aqueous layer;
(xvii) acidifying the organic layer with a mineral acid solution selected from HCl and H₂SO₄ to partition therefrom an organic layer containing the highly purified CBG or CBCV or CBDV or THCV or CBC in the form of an oil, and an aqueous layer containing the amine as its hydrochloride;
(xviii) dewatering and drying the purified CBG or THCV or CBDVA or CBCVA or CBCA organic layer; and
(xix) removing the fourth organic solvent from the highly purified CBG or CBCV or CBDV or THCV or CBC.

103. The method according to claim 100, wherein the CBGA-amine salt is precipitated with one of N,N-diisopropylethylamine (Hunig's base), dicyclohexylamine, methyldicyclohexyamine, 1,4-diazabicyclo[2.2.2]-octane (DABCO), triethylamine, tripropylamine, tributylamine, isopropylcyclohexylamine, and 2,2,6,6-tetramethylpiperidine.

104. The method according to claim 100, wherein the THCVA-amine salt is precipitated with one of dimethylethanolamine (DMEA), 1,5-diazabicyclo(4.3.0)non-5-ene (DBN), and cyclohexylisopropylamine.

105. The method according to claim 100, wherein the CBDVA-amine salt is precipitated with one of triethylamine, N,N-diisopropylethylamine (Hunig's base), 1,5-diazabicyclo(4.3.0)non-5-ene (DBN), and dimethylethanolamine (DMEA).

106. The method according to claim 100, wherein the CBDVA-amine salt is precipitated at a temperature of 0° C. or less, with one of tetramethylethylenediamine, methylpiperazine, and cyclohexylisopropylamine.

107. The method according to claim 100, wherein the CBCVA-amine salt is precipitated with one of isopropylcyclohexylamine, 2,2,6,6-tetramethylpiperidine, dicyclohexylamine, N,N-diisopropylethylamine (Hunig's base), 1,5-diazabicyclo(4.3.0)non-5-ene salt (DBN), 1,4-diazabicyclo[2.2.2]octane (DABCO), and dimethylaminopyridine (DMAP).

108. The method according to claim 100, wherein the CBCA-amine salt is precipitated with one of 1,5-diazabicyclo(4.3.0)non-5-ene (DBN) and 4-dimethylaminopyridine.

109. The method according to claim 103, wherein:
(i) the amine is N,N-diisopropylethylamine and the salt produced is a CBGA-N,N-diisopropylethylamine salt; or
(ii) the amine is dicyclohexylamine and the salt produced is a CBGA-dicyclohexylamine salt; or
(iii) the amine is N,N-methyldicyclohexyamine and the salt produced is a CBGA-N,N-methyldicyclohexyamine salt; or
(iv) the amine is 1,4-diazabicyclo[2.2.2]octane and the salt produced is a CBGA-1,4-diazabicyclo[2.2.2]octane salt; or
(v) the amine is triethylamine and the salt produced is a CBGA-triethylamine salt; or
(vi) the amine is tripropylamine and the salt produced is a CBGA-tripropylamine salt; or
(vii) the amine is tributylamine and the salt produced is a CBGA-tributylamine salt; or
(viii) the amine is isopropylcyclohexylamine and the salt produced is a CBGA-isopropylcyclohexylamine salt; or
(ix) the amine is 2,2,6,6-tetramethylpiperidine and the salt produced is a CBGA-2,2,6,6-tetramethylpiperidine salt.

110. The method according to claim 104, wherein:
(i) the amine is dimethylethanolamine and the salt produced is a THCVA-dimethylethanolamine salt; or
(ii) the amine is 1,5-diazabicyclo(4.3.0)non-5-ene and the salt produced is a THCVA-1,5-diazabicyclo(4.3.0)non-5-ene salt; or
(iii) the amine is cyclohexylisopropylamine and the salt produced is a THCVA-cyclohexylisopropylamine salt.

111. The method according to claim 105 or 106, wherein:
(i) the amine is triethylamine and the salt produced is a CBDVA-triethylamine salt; or
(ii) the amine is N,N-diisopropylethylamine and the salt produced is a CBDVA-N,N-diisopropylethylamine salt; or
(iii) the amine is 1,5-diazabicyclo(4.3.0)non-5-ene and the salt produced is a CBDVA-1,5-diazabicyclo(4.3.0)non-5-ene salt; or
(iv) the amine is dimethylethanolamine and the salt produced is a CBDVA-dimethylethanolamine salt; or
(v) the amine is cyclohexylisopropylamine and the salt produced is a CBDVA-cyclohexylisopropylamine salt; or
(vi) the amine is tetramethylethylenediamine and the salt produced is a CBDVA-tetramethylethylenediamine salt; or
(vii) the amine is methylpiperazine and the salt produced is a CBDVA-methylpiperazine salt.

112. The method according to claim 107, wherein:
(i) the amine is isopropylcyclohexylamine and the salt produced is a CBCVA-isopropylcyclohexylamine salt; or
(ii) the amine is 2,2,6,6-tetramethylpiperidine and the salt produced is a CBCVA-2,2,6,6-tetramethylpiperidine salt; or
(iii) the amine is dicyclohexylamine and the salt produced is a CBCVA-dicyclohexylamine salt; or
(iv) the amine is N,N-diisopropylethylamine and the salt produced is a CBCVA-N,N-diisopropylethylamine salt; or
(v) the amine is 1,4-diazabicyclo[2.2.2]octane and the salt produced is a CBCVA-1,4-diazabicyclo[2.2.2]octane salt; or
(vi) the amine is dimethylaminopyridine and the salt produced is a CBCVA-dimethylaminopyridine salt.

113. The method according to claim 108, wherein:
(i) the amine is 1,5-diazabicyclo(4.3.0)non-5-ene and the salt produced is a CBCA-1,5-diazabicyclo(4.3.0)non-5-ene salt; or
(ii) the amine is 4-dimethylaminopyridine and the salt produced is a CBCA-4-dimethylaminopyridine salt.

114. A cannabigerolic acid-amine salt having:
(i) a chemical structure 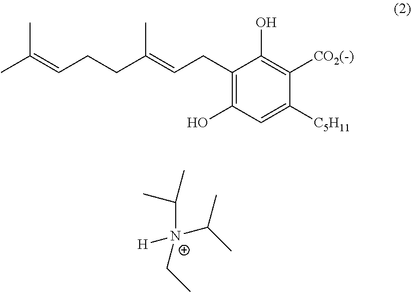
(CBGA-N,N-diisopropylethylamine salt); or
(ii) a chemical structure 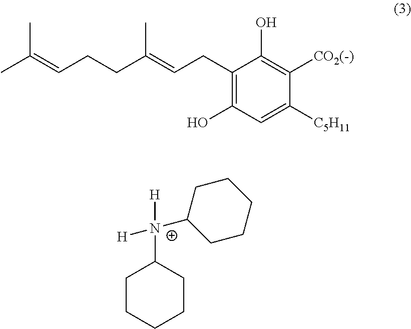
(CBGA-dicyclohexylamine salt); or
(iii) a chemical structure 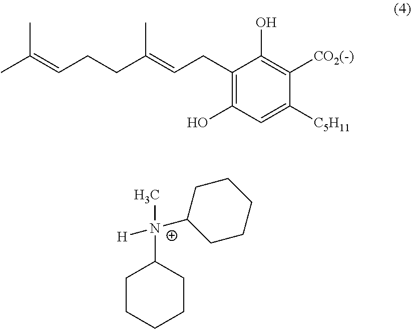
(CBGA-methyldicyclohexyamine salt); or
(iv) a chemical structure 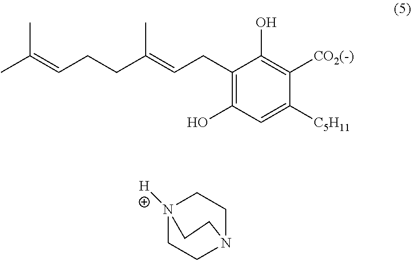
(CBGA-1,4-diazabicyclo[2.2.2]octane salt); or
(v) a chemical structure 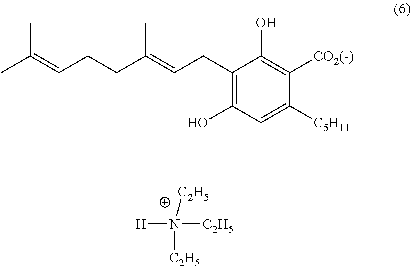
(CBGA-triethylamine salt); or
(vi) a chemical structure 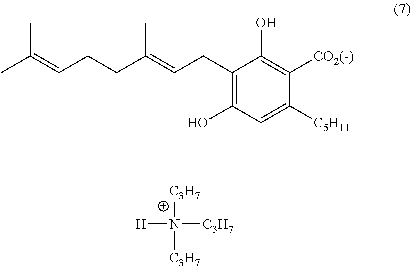
(CBGA-tripropylamine salt); or
(vii) a chemical structure 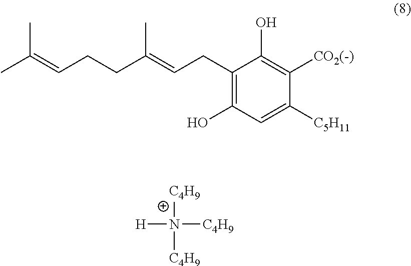
(CBGA-tributylamine salt); or
(viii) a chemical structure 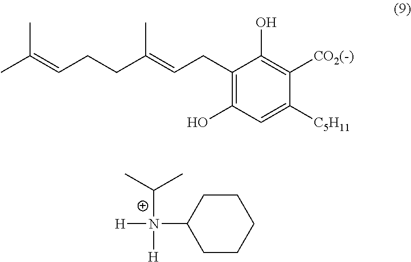
(CBGA-isopropycyclohexylamine salt); or
(ix) a chemical structure 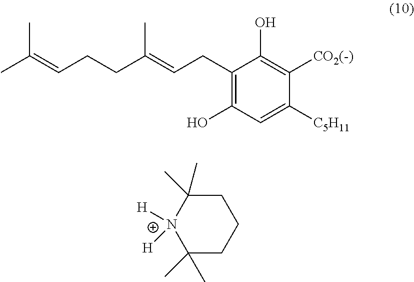
(CBGA-2,2,6,6-tetramethylpiperidine).

115. A cannabidivarinic acid-amine salt having:
(i) a chemical structure 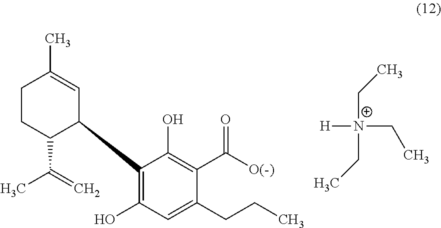
(CBDVA-triethylamine salt); or
(ii) a chemical structure 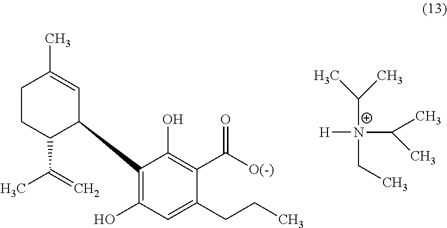
(CBDVA-N,N-diisopropylethylamine salt); or
(iii) a chemical structure 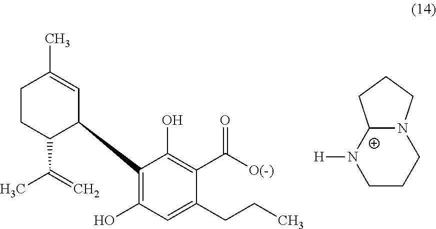
(CBDVA-1,5-diazabicyclo(4.3.0)non-5-ene salt); or
(iv) a chemical structure 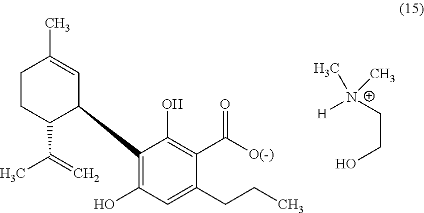
(CBDVA-dimethylethanolamine salt); or
(v) a chemical structure 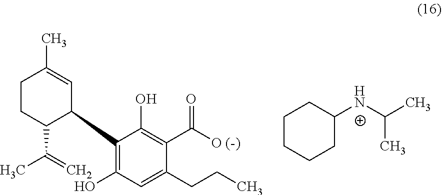
(CBDVA-cyclohexylisopropylamine salt); or
(vi) a chemical structure 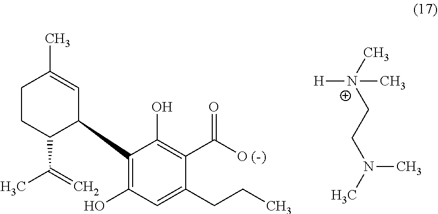
(CBDVA-tetramethylethylenediamine salt); or
(vii) a chemical structure 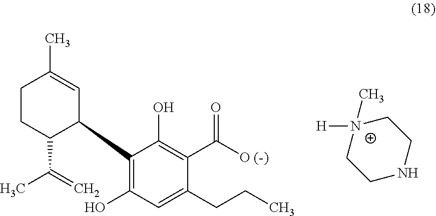
(CBDVA-methylpiperazine amine salt).

116. A tetrahydrocannabivirinic acid-amine salt having:
(i) a chemical structure 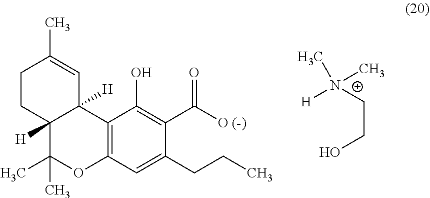
(THCVA-dimethylethanolamine salt); or
(ii) a chemical structure 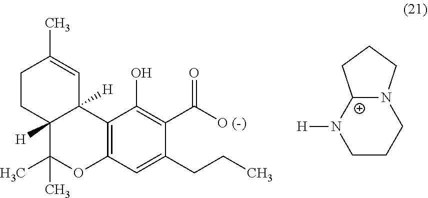
(THCVA-1,5-diazabicyclo(4.3.0)non-5-ene salt); or
(iii) a chemical structure 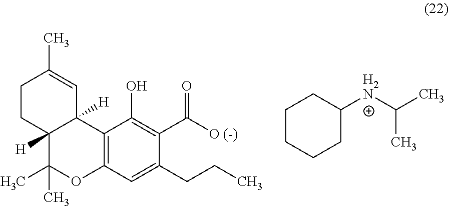
(THCVA-cyclohexylisopropylamine salt);

117. A cannabichromevarinic acid-amine salt having:
a chemical structure 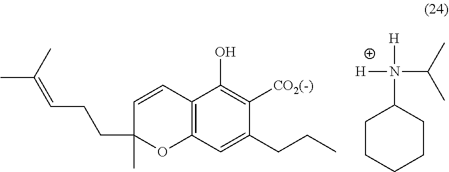
(CBCVA-isopropylcyclohexylamine salt); or
(ii) a chemical structure 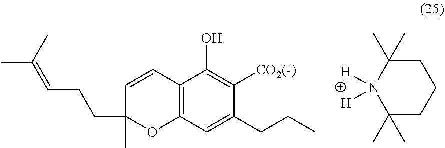
(CBCVA-2,2,6,6-tetramethylpiperidine salt); or
(iii) a chemical structure 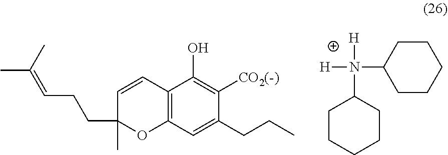
(CBCVA-N,N-dicyclohexylamine amine salt); or
(iv) a chemical structure 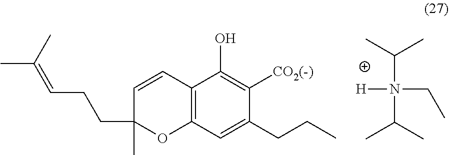
(CBCVA-N,N-diisopropylethylamine salt (Hunig's base)); or
(v) a chemical structure 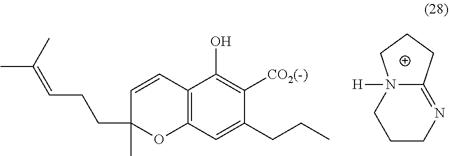
(CBCVA 1,5-diazabicyclo[4.3.0]non-5-ene (DBN)); or
(vi) a chemical structure 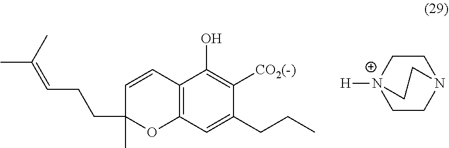
(CBCVA-1,4-diazabicyclo[2.2.2]octane salt (DABCO)); or
(vii) a chemical structure 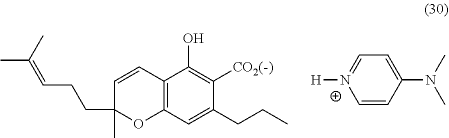
(CBCVA-4-dimethylaminopyridine(DMAP)).

118. A cannabichromic acid-amine salt having:
(i) a chemical structure 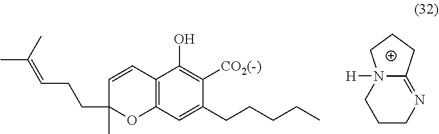
(CBCA-1,5-diazabicyclo[4.3.0]non-5-ene salt); or
(ii) a chemical structure 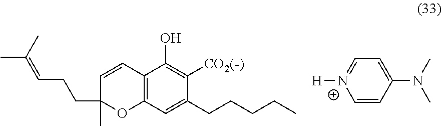
(CBCA-4-dimethylaminopyridine salt).
